SEQUENCE LISTING (153 sequences)

SEQ ID NO: 1 - DNA, 1173 bp, Chlamydomonas reinhardtii
atgcagatgatgcagcgcaacgttgtgggccagcgccccgtcgctggctcccgccgctcgctggtggttgccaacgttgcggaggtgacccgccccgcggtcagcaccaacggcaagcaccggactggtgtgccggagggaactcccatcgtcacccctcaggacctgccctcgcgccctcgccgcaaccgccgcagcgagagcttccgtgcttccgttcgtgaggtgaacgtgtcgcccgccaacttcatcctgccgatcttcatccacgaggagagcaaccagaacgtgcccatcgcctccatgcctggcatcaaccgcctggcgtatggcaagaacgtgattgactacgttgctgaggctcgctcttacggtgtcaaccaggtcgtggttttccccaagacgcccgaccacctgaagacgcaaaccgcggaggaggcgttcaacaagaacggcctcagccagcgcacgatccgcctgctgaaggactctttccctgacctggaggtgtacacggacgtggctctggacccctacaactcggacggccacgacggtatcgtgtcggacgccggtgtgatcctgaacgacgagaccatcgagtacctgtgccgccaggccgtgagccaggccgaggccggtgccgacgtggtgtcgccctctgacatgatggacggccgcgtgggcgccatccgccgcgccctggaccgcgagggcttcaccaacgtgtccatcatgtcctacaccgccaagtacgcctccgcctactacggccccttccgtgacgccctggcgtccgcgcccaagcccggccaggcgcaccgccgcatcccccccaacaagaagacctaccagatggaccccgccaactaccgcgaggccatccgcgaggccaaggccgacgaggccgagggcgctgacatcatgatggtcaagcccggcatgccgtacctggacgtggtacgcctgctgcgtgagaccagcccgctgcccgtggccgtgtaccacgtgtcgggcgagtacgccatgctcaaggcggcggcggagcgcggctggctgaacgagaaggatgccgtgcttgaggccatgacctgcttccgccgcgccggcgctgacctcatcctcacctactacggcattgaggcctccaagtggctggcgggcgagaagtaa

SEQ ID NO: 2 - protein, 390 aa, Chlamydomonas reinhardtii
MQMMQRNVVGQRPVAGSRRSLVVANVAEVTRPAVSTNGKHRTGVPEGTPIVTPQDLPSRPRRNRRSESFRASVREVNVSPANFILPIFIHEESNQNVPIASMPGINRLAYGKNVIDYVAEARSYGVNQVVVFPKTPDHLKTQTAEEAFNKNGLSQRTIRLLKDSFPDLEVYTDVALDPYNSDGHDGIVSDAGVILNDETIEYLCRQAVSQAEAGADVVSPSDMMDGRVGAIRRALDREGFTNVSIMSYTAKYASAYYGPFRDALASAPKPGQAHRRIPPNKKTYQMDPANYREAIREAKADEAEGADIMMVKPGMPYLDVVRLLRETSPLPVAVYHVSGEYAMLKAAAERGWLNEKDAVLEAMTCFRRAGADLILTYYGIEASKWLAGEK

SEQ ID NO: 3 - DNA, 1098 bp, Chlamydomonas reinhardtii
atggcactgcaagcctcaacccgctcgctccagcagcgccgcgccttctcttcggcccagacctccaagcgtgtgtctgtgaccaaggtccgcgcgacggctatcgaggcggagaactatgtgaagcaggctccccagtcgctggtccgcccgggcatcgacactgaggactctatgcgcgctcgcttcgagaaggtgatccgcaacgcccaggactccatctgcaatgctatctccgagatcgatggcaagccgttccaccaggacgcctggacccgccccggcggcggtggcggcatcagccgcgtgctgcaggacggcaacgtgtgggagaaggccggcgtcaacgtgtccgtggtctacggcaccatgccccctgaggcctaccgcgctgccactggcaacgccgagaagctgaagaacaagggtgacggtggccgcgtgcccttcttcgccgccggcatctcgtcggtgatgcacccccgcaacccccactgccccaccatgcacttcaactaccgctacttcgagactgaggagtggaacggcatccccggccagtggtggttcggcggcggcaccgacatcacccccagctatgtggtgcccgaggacatgaagcacttccacggcacctacaaggcggtgtgcgaccgccacgatcccgcttactacgagaagttccgcacctggtgcgatgagtacttcctcatcaagcaccgcggcgagcgccgcggcctgggcggcatcttcttcgatgacctgaacgaccgcaaccccgaggacatcctgaagttctcgaccgacgccgtgaacaacgtggtggaggcatactgccccatcatcaagaagcacatgaacgacccctacacccccgaggagaaggagtggcagcagatccgccgcggccgctacgtggagttcaacctggtctatgaccgcggcaccaccttcggcctgaagaccggcggccgcattgagtcgatcctcatgtccatgccccagaccgcctcatggctgtacgaccaccagcccaaggccggctcgcccgaggccgagctgctcgacgcctgccgcaacccccgcgtctgggtgtaa

SEQ ID NO: 4 - protein, 365 aa, Chlamydomonas reinhardtii
MALQASTRSLQQRRAFSSAQTSKRVSVTKVRATAIEAENYVKQAPQSLVRPGIDTEDSMRARFEKVIRNAQDSICNAISEIDGKPFHQDAWTRPGGGGGISRVLQDGNVWEKAGVNVSVVYGTMPPEAYRAATGNAEKLKNKGDGGRVPFFAAGISSVMHPRNPHCPTMHFNYRYFETEEWNGIPGQWWFGGGTDITPSYVVPEDMKHFHGTYKAVCDRHDPAYYEKFRTWCDEYFLIKHRGERRGLGGIFFDDLNDRNPEDILKFSTDAVNNVVEAYCPIIKKHMNDPYTPEEKEWQQIRRGRYVEFNLVYDRGTTFGLKTGGRIESILMSMPQTASWLYDHQPKAGSPEAELLDACRNPRVWV

SEQ ID NO: 5 - DNA, 1049 bp, Chlamydomonas reinhardtii
atgctgaggaagcagattggtggatctggccagcagcgggcgggcctccgacgggtgaaccaaggacctgcgcgtcggcggttggcaccctgccgcgtggcggcccccgtgcaaacctcgtcctccgtcgccacattcaatggcttcgtggactacattcacggactccagaagaacattctgagcactgctgaggatctggagaacggcgagcggaagtttgttgttgaccgctgggagcgcgacgccagcaaccccaacgccgggtatggcattacgtgcgtgcttgaggacgggaaggtgctggagaaggccgcagccaatatctcagtggtgcgcgggacgctgtcggcgcagcgcgcagtggccatgagctcccgcggccgcagcagcatcgaccccaagggcgggcagccctacgccgcggccgccatgagcctagtgttccacagcgcgcacccgctcatccccacgctgcgcgcgacgtgcggttgttccaggtgggcgatgaggcgtggtacggcggtggctgtgacctgacgcccaactacctagacgtggaggactcgcagtccttccaccgctactggaaggacgtgtgcggcaagtacaagccgggcctgtacaccgagctcaaggagtggtgcgacaggtacttctacatcccggcccgcaaagagcaccgtggcattggcggcctgttctttgatgacatggccactgcggaggcgggctgcgatgtggaggcgtttgtgcgggaagtgggagatggcatcctgccctgctggctgcccatcgtggcgcggcaccgtggccagcccttcacggagcagcagcggcaatggcagctgctgcgccgcggtcgctacatcgagttcaacctgctgtacgaccgcggcatcaagttcggtctggacggcggccgcatcgagagcatcatggtgtcggcgccgccgctgatcgcgtggaagtacaacgtggtgccacagccgggcagccccgaggaggagatgctgaaggtgcttcagcagccccgcgagtgggcctga

SEQ ID NO: 6 - protein, 349 aa, Chlamydomonas reinhardtii
MLRKQIGGSGQQRAGLRRVNQGPARRRLAPCRVAAPVQTSSSVATFNGFVDYIHGLQKNILSTAEDLENGERKFVVDRWERDASNPNAGYGITCVLEDGKVLEKAAANISVVRGTLSAQRAVAMSSRGRSSIDPKGGQPYAAAAMSLVFHSAHPLIPTLRADVRLFQVGDEAWYGGGCDLTPNYLDVEDSQSFHRYWKDVCGKYKPGLYTELKEWCDRYFYIPARKEHRGIGGLFFDDMATAEAGCDVEAFVREVGDGILPCWLPIVARHRGQPFTEQQRQWQLLRRGRYIEFNLLYDRGIKFGLDGGRIESIMVSAPPLIAWKYNVVPQPGSPEEEMLKVLQQPREWA

SEQ ID NO: 7 - DNA, 1482 bp, Chlamydomonas reinhardtii
atggcgtcgtttggattgatgcaaaggacggtgcactgtccccagcttgtggaggagcggtgttcgccggtcgctggctgctctggtcgtggcctgccagttatccagcggcaacggcgtggcgtgtgcagtgccaccaacggtgtccagcgagggcgtgtgctgcgccggacggccgcttcgaccgacgtggtctccttcgtggaccccaatgacattagaaaacccgcagcagcagcagctggccctgcggtggataaggtcggcgttctgctgttaaaccttggcgggcccgaaaagctcgacgacgtcaagcctttcctgtataacctattcgccgacccagaaattattcgcctgccagcggcagctcagttcctgcagccgctgctcgcgacgatcatctccacgcttcgcgccccgaagagcgcggagggctatgaggccattggcggtggtagcccgttgcgtaggattacagacgagcaggcggaggcgctggcggagtctctgcgcgccaagggccaacctgcgaacgtgtacgtgggcatgcgctattggcacccctacacggaggaggcgctggagcacattaaggccgacggcgtcacgcgcctggtcatcctcccgctgtaccctcagttctccatctctaccagcggctccagccttcgactgcttgagtcgctcttcaagagcgacatcgcgctcaagtcgctgcggcacacggtcatcccgtcctggtaccagcggcggggctacgtgagcgcgatggcggacctgattgtagaggagctgaagaagttccgggacgtgcccagcgtggagctgtttttctccgcgcacggcgtgcccaagtcctacgtggaggaggcgggcgacccatacaaggaggagatggaggagtgcgtgcggctcattacggacgaggtcaagcggcgcggcttcgccaacacgcacacgctggcctaccagagccgcgtgggccccgcggaatggctcaagccgtacacggatgagtccatcaaggagctgggcaagcgcggcgtcaagtcgctgctggcggtgcccatcagctttgtcagcgagcacattgagacgttggaggagatcgacatggagtaccgcgagctggcggaggagagcggcatccgcaactggggccgcgtgccggcgctgaacaccaacgccgccttcatcgacgacctggcggacgcggtgatggaggcgctgccctacgtgggctgcctggccgggccgacagactcgctggtgccgctgggcgacctggagatgctgctgcaggcctacgaccgcgagcgccgcacgctgccgtcaccggtggtgatgtgggagtggggctggaccaagagcgcggagacgtggaacggccgcattgccatgattgccatcatcatcatcctggcgctggaggcagccagcggccagtccatcctcaaaaacctgttcctggcggagtag

SEQ ID NO: 8 - protein, 492 aa, Chlamydomonas reinhardtii
MASFGLMQRTVHCPQLVEERCSPVAGCSGRGLPVIQRQRRGVCSATNGVQRGRVLRRTAASTDVVSFVDPNDIRKPAAAAAGPAVDKVGVLLLNLGGPEKLDDVKPFLYNLFADPEIIRLPAAAQFLQPLLATIISTLRAPKSAEGYEAIGGGSPLRRITDEQAEALAESLRAKGQPANVYVGMRYWHPYTEEALEHIKADGVTRLVILPLYPQFSISTSGSSLRLLESLFKSDIALKSLRHTVIPSWYQRRGYVSAMADLIVEELKKFRDVPSVELFFSAHGVPKSYVEEAGDPYKEEMEECVRLITDEVKRRGFANTHTLAYQSRVGPAEWLKPYTDESIKELGKRGVKSLLAVPISFVSEHIETLEEIDMEYRELAEESGIRNWGRVPALNTNAAFIDDLADAVMEALPYVGCLAGPTDSLVPLGDLEMLLQAYDRERRTLPSPVVWEWGWTKSAETWNGRIAMIAIIIILALEAASGQSILKNLFLAE

SEQ ID NO: 9 - DNA, 1392 bp, Chlamydomonas reinhardtii
atgcagatgcagctgaacgccaagaccgtgcagggcgccttcaaggcgcagcgccctcgctctgtccgcggcaacgtggcggtgcgcgcagtggccgctccccctaagctggtcaccaagcgctccgaggagatcttcaaggaggctcaggagctgctgcccggtggcgtgaactcgcccgtgcgcgctttccgctcggttggtggcggccccatcgtcttcgacagggtcaagggtgcctactgctgggacgtcgatggcaacaagtacatcgactacgttggctcttggggccctgccatttgcggccacggcaacgacgaggtcaacaacgccctgaaggcgcagatcgacaagggcacctcgttcggtgctccctgcgagctggagaacgtgctggccaagatggtgattgaccgcgtgccctcggtggagatggtgcgcttcgtgtcctcgggcactgaggcgtgcctgtcggtgctgcgcctgatgcgcgcatacaccggccgcgagaaggtgctgaagttcaccggctgctaccacggccacgccgactccttcctggtgaaggccggctccggtgtgatcaccctgggcctgcccgactcgcccggtgtgcccaagagcaccgccgccgccaccctgaccgccacctacaacaacctggactccgtgcgcgagctgttcgccgccaacaagggcgagattgccggtgtgatcctggagcccgtggtcggcaacagcggcttcattgtgcccaccaaggagttcctgcagggcctgcgcgagatctgcacggctgagggcgccgtgctgtgcttcgatgaggtcatgaccggcttccgcattgccaagggctgcgcccaggagcacttcggtatcacccccgacctgaccaccatgggcaaggtcattggtggcggcatgcctgtgggcgcctacggcggcaagaaggagatcatgaagatggtcgcccccgccggccccatgtaccaggccggcaccctttcgggcaaccccatggccatgactgccggcatcaagacgctggagatcctgggccgccccggcgcctacgagcacctggagaaggtgaccaagcgcctgatcgacggcatcatggccgccgccaaggagcacagccacgagatcaccggcggcaacatcagcggcatgtttggcttcttcttctgcaagggccctgtgacctgcttcgaggacgccctggcggccgacactgccaagttcgcgcgcttccaccgcggcatgctggaggagggcgtctacctggctccctcgcagttcgaggccggcttcacctctctggcccactccgaggcggacgtggatgccacgatcgccgccgctcgccgcgtgttcgcccgcatctaa

SEQ ID NO: 10 - protein, 463 aa, Chlamydomonas reinhardtii
MQMQLNAKTVQGAFKAQRPRSVRGNVAVRAVAAPPKLVTKRSEEIFKEAQELLPGGVNSPVRAFRSVGGGPIVFDRVKGAYCWDVDGNKYIDYVGSWGPAICGHGNDEVNNALKAQIDKGTSFGAPCELENVLAKMVIDRVPSVEMVRFVSSGTEACLSVLRLMRAYTGREKVLKFTGCYHGHADSFLVKAGSGVITLGLPDSPGVPKSTAAATLTATYNNLDSVRELFAANKGEIAGVILEPVVGNSGFIVPTKEFLQGLREICTAEGAVLCFDEVMTGFRIAKGCAQEHFGITPDLTTMGKVIGGGMPVGAYGGKKEIMKMVAPAGPMYQAGTLSGNPMAMTAGIKTLEILGRPGAYEHLEKVTKRLIDGIMAAAKEHSHEITGGNISGMFGFFFCKGPVTCFEDALAADTAKFARFHRGMLEEGVYLAPSQFEAGFTSLAHSEADVDATIAAARRVFARI

SEQ ID NO: 11 - DNA, 1569 bp, Chlamydomonas reinhardtii
atgcagaccactatgcagcagcgtctccagggccgtaacgtggccgggcggagcgtcgctccctcggtccctgcccatcgctccttccactcacaccgggctgccactcaaaccgctacgatcagcgctgctgctagctcaaccaccaagctgccagcttcgcatctggagagcagcaagaaggcgctggattcgctgaagcagcaggccgtcaatcgctacgcgggtgacaagaagagctccattattgccattggtctcaccattcacaacgcacccgtggagctgcgcgagaagctggctgtgcctgaggctgaatggccgcgtgctattgaggagctctgccagttcccgcacatcgaggaggccgcggtgctgtcgacgtgcaatcgcatggagctctacgttgtcggtctgtcgtggcaccgcggcgttcgcgaggtggaggagtggctgtctcgcaccagcggcgtgcctctggatgagctgcgcccctacctgttcctgctgcgcgaccgcgacgccacgcaccacctgatgcgcgtgtcgggtggccttgactcgctggttatgggcgagggccagattctcgcccaagtgcgccaggtctacaaggtcggccagaactgccccggcttcggtcgccacctgaacggcctgttcaagcaggctatcaccgctggcaagcgcgtgcgtgccgagacctccatctccaccggctccgtctccgtctcatccgccgccgtcgagctggcgcagctcaagctccccacccacaactggtccgacgctaaggtctgcatcatcggcgctggcaagatgtctacgctgctggtgaagcacctgcagagcaagggctgcaaggaggtgacggtgctcaaccgctctctgccgcgcgcccaggcgctggcggaggagttccctgaggtcaagttcaacatccacctgatgcccgacctgctgcagtgcgtggaggccagcgacgtcatcttcgccgcctccggctctgaggagatcctcatccacaaggagcatgtcgaggccatgtccaagccatcggacgttgttggctccaagcgccgcttcgtcgacatctccgtgccccgcaacatcgcccccgccatcaacgagctggagcacggcatcgtctacaacgtcgacgacctgaaggaggttgtggccgccaacaaggagggccgcgcgcaggcggccgccgaggccgaggtgctgatccgcgaggagcagcgcgcgttcgaggcctggcgtgactctctggagaccgtgcccaccatcaaggcgctgcgctccaaggccgagaccatccgcgccgccgagtttgagaaggccgtgtctcgcctgggcgaggggctatccaagaagcagctcaaggcggtggaggagctcagcaagggcatcgtcaacaagctgctgcacgggcccatgacggcactgcgctgcgacggcaccgatccggatgccgtgggccagaccctcgcgaacatggaggccctggagcgcatgttccagctctcggaggtggacgtggccgcgctggcgggcaagcagtaa

SEQ ID NO: 12 - protein, 522 aa, Chlamydomonas reinhardtii
MQTTMQQRLQGRNVAGRSVAPSVPAHRSFHSHRAATQTATISAAASSTTKLPASHLESSKKALDSLKQQAVNRYAGDKKSSIIAIGLTIHNAPVELREKLAVPEAEWPRAIEELCQFPHIEEAAVLSTCNRMELYVVGLSWHRGVREVEEWLSRTSGVPLDELRPYLFLLRDRDATHHLMRVSGGLDSLVMGEGQILAQVRQVYKVGQNCPGFGRHLNGLFKQAITAGKRVRAETSISTGSVSVSSAAVELAQLKLPTHNWSDAKVCIIGAGKMSTLLVKHLQSKGCKEVTVLNRSLPRAQALAEEFPEVKFNIHLMPDLLQCVEASDVIFAASGSEEILIHKEHVEAMSKPSDVVGSKRRFVDISVPRNIAPAINELEHGIVYNVDDLKEVVAANKEGRAQAAAEAEVLIREEQRAFEAWRDSLETVPTIKALRSKAETIRAAEFEKAVSRLGEGLSKKQLKAVEELSKGIVNKLLHGPMTALRCDGTDPDAVGQTLANMEALERMFQLSEVDVAALAGKQ

SEQ ID NO: 13 - DNA, 1779 bp, Chlamydomonas reinhardtii
atgttatactcacaatttaaacattcggtgcctttaggccgtaagtctccccttctttcagggggccccccttctgggggtcgcccaacaacggctgcctcaggcctaggtcgcaacgtggccgtaagaattgggaccccgttgggctttgcccttcgggcccaggtaattatggcagctgcgggcaatactagcggtgcgccgcaccccgtaggggagtcccagcctgcgttgtcccaggtggattctcaacttgtaattgagtgtgaaacaggaaattaccatactttttgcccaattagttgtgtttcttggttataccaaaaaattgaagatagttttttcttagttattggtacaaaaacgtgtgggtattttttacaaaatgctttaggggttatgatttttgccgaacctcgttacgctatggcggaattagaagaaagcgatatttcggcgcaattaaatgattacaaagaattaaaacgtctatgtttacaaattaaacaagaccgtaacccaagtgttattgtgtggattggcacatgcacaaccgaaattattaaaatggatttagaaggtatggcaccgaaactagaagctgaaatcggtattccaattgtggtagcacgcgcaaatggacttgattatgcttttacacaaggtgaagatactgttttagctgcgatggtccaaaaatgcccggaattaggcgctattccagctattgtacctcagattccttctgactctcgtacacttagccaactatctgtagcggcttcggtacccgaaaacagtgcgtctgggccagaaggggagccttcactagcccagaagggaatggattctaagttaacaaacaactctccatgccgagtagattctgtctcagaatctaccccggcgtttcctggacgtgctccgcacgtcgggaaaagtactcctcaaaatttagttttatttggttcattacctagcacgatggcaaatcaactggagtttgaattaaaacgccaaggtattaatgttactgggtggttacctgcggctcgctattcatctttacctgcattaggtgaaaacgtgtatgtttgtgggattaatccatttttaagtcgaactgctacttctttaatgcgtcgtcgtaaatgcaaattaatttcagctcctttcccaattggtccagatggtacaaaagcttgggtcgaaaaaatttgtaatgttttcggtgttacaccaactggtttagaagatcgtgaacgtcttgtttgggaaggtttaaaagattatttaaatttcgtaaaagggaaatctgttttctttatgggtgataatctgttagaaatttcattagcccgttttttaattcgctgtggtatgaccgtttatgaaatcggtattccgtacatggaccaacgatttcaagctggggaattagaattattaaaaaaaacatgcatggaaatgaacgtgcccctaccgcgtattgttgaaaaacctgataattactatcaaattcaacgtattaaagaattacaaccagatttagttattaccggcatggcccatgcaaacccactggaagcgcgcggcattactacgaaatggtccgttgaatttacgtttgcgcaaattcatgggtttggcaacgcacgtgatatcttagaattagttacaaaaccgttacgtcgtaataaaaatctatctaaatatcaatttccgttagatagctgggacaagcctgcttccgtaggcgctcacgaactgtcggcctaa

SEQ ID NO: 14 - protein, 592 aa, Chlamydomonas reinhardtii
MLYSQFKHSVPLGRKSPLLSGGPPSGGRPTTAASGLGRNVAVRIGTPLGFALRAQVIMAAAGNTSGAPHPVGESQPALSQVDSQLVIECETGNYHTFCPISCVSWLYQKIEDSFFLVIGTKTCGYFLQNALGVMIFAEPRYAMAELEESDISAQLNDYKELKRLCLQIKQDRNPSVIVWIGTCTTEIIKMDLEGMAPKLEAEIGIPIVVARANGLDYAFTQGEDTVLAAMVQKCPELGAIPAIVPQIPSDSRTLSQLSVAASVPENSASGPEGEPSLAQKGMDSKLTNNSPCRVDSVSESTPAFPGRAPHVGKSTPQNLVLFGSLPSTMANQLEFELKRQGINVTGWLPAARYSSLPALGENVYVCGINPFLSRTATSLMRRRKCKLISAPFPIGPDGTKAWVEKICNVFGVTPTGLEDRERLVWEGLKDYLNFVKGKSVFFMGDNLLEISLARFLIRCGMTVYEIGIPYMDQRFQAGELELLKKTCMEMNVPLPRIVEKPDNYYQIQRIKELQPDLVITGMAHANPLEARGITTKWSVEFTFAQIHGFGNARDILELVTKPLRRNKNLSKYQFPLDSWDKPASVGAHELSA

SEQ ID NO: 15 - DNA, 1644 bp, Chlamydomonas reinhardtii
atgaaattagcgtattggatgtatgcgggaccggctcatattggaacattacgagttgcaagctcgtttcgaaatgtgcatgctattatgcatgctcccttaggcgatgattattttaacgtaatgcgttcaatgttagaacgtgaacgtgattttacgccagtgacggcaagtattgttgatcgtcatgttttagctcgtggttcacaagaaaaagttgttgaaaacattcaacgaaaagataaagaagaatgtccggatttaattttattaacaccaacatgtacctcaagtattttgcaagaagatttacaaaattttgtaaatcgcgcggccgaagtagcaaagcgttcggatgttttattagctgacgttaaccattaccgagtgaatgaattacaagcggctgaccgtacgttagagcaaattgtacgcttttatttagaaaaagaagtaaataaacttcacgcggagttaggcggccttaaaaaaccgcttcgctttgcccagcgtacccaaaagccgtctgccaatattttaggcatgtttacactaggtttccataatcaacatgactgtcgtgaattaaaacgtttattaaatgatttaggtatcgaagtcaatgaagtgattcctgaaggtagttttgtacatggattaaaaaatttaccaaaagcgtggtttaacatcgtcccgtatcgtgaagttggtttaatgacggcaatttatttagaaaaagaatttggcatgccttatacctcaatcacgccaatgggcattattgacaccgcggcgtttattcgtgaaattgcggccatttgtagtcaaattagcacttcacaggcatctacaaactcaactgaaggactccagaggggagaaaatgtcagtttaactgaaactaattcgattatttttaataaagcaaaatatgaacaatacattaatcaacaaacgcattttgtttctcaagcagcttggttttcacgttctattgactgtcaaaatttaaccggtaaaaaaaccgttgtgtttggtgatgcaactcacgcggcaagtatgacgaaaattcttgtgcgcgaaatgggtattcatgttgtttgcgcgggcacgtattgtaaacatgatgcagattggtttagagagcaagtttcaggtttttgtgatcaagttttaattacagatgatcacagccaaattgcggaaatcattgctcaaattgaacctgcagccatttttggtacacaaatggaacgtcatgttgggaaaaggttagatattccttgtggggttatttctgcaccggtacatattcaaaacttcccactaggctttagaccgtttttagggtatgaaggtactaatcaaatttccgatttagtttataattcgtttagtttaggtatggaagatcacttactagaaattttcaacggtcatgacaataaagaagttattacacgttcgtattcttcagaaactgatttagaatggacaaaagaagcattagatgaactagctcgtgttcctggttttgttcgttcaaaagttaaacgtaatactgaaaaatttgcgcgtacaaataaaaatcaagttattactattgaagttatgtacgcagctaaagaagcggtatcagcgtaa

SEQ ID NO: 16 - protein, 547 aa, Chlamydomonas reinhardtii
MKLAYWMYAGPAHIGTLRVASSFRNVHAIMHAPLGDDYFNVMRSMLERERDFTPVTASIVDRHVLARGSQEKVVENIQRKDKEECPDLILLTPTCTSSILQEDLQNFVNRAAEVAKRSDVLLADVNHYRVNELQAADRTLEQIVRFYLEKEVNKLHAELGGLKKPLRFAQRTQKPSANILGMFTLGFHNQHDCRELKRLLNDLGIEVNEVIPEGSFVHGLKNLPKAWFNIVPYREVGLMTAIYLEKEFGMPYTSITPMGIIDTAAFIREIAAICSQISTSQASTNSTEGLQRGENVSLTETNSIIFNKAKYEQYINQQTHFVSQAAWFSRSIDCQNLTGKKTVVFGDATHAASMTKILVREMGIHVVCAGTYCKHDADWFREQVSGFCDQVLITDDHSQIAEIIAQIEPAAIFGTQMERHVGKRLDIPCGVISAPVHIQNFPLGFRPFLGYEGTNQISDLVYNSFSLGMEDHLLEIFNGHDNKEVITRSYSSETDLEWTKEALDELARVPGFVRSKVKRNTEKFARTNKNQVITIEVMYAAKEAVSA

SEQ ID NO: 17 - DNA, 957 bp, Chlamydomonas reinhardtii
atgaaattagcagtttatggcaaaggtggtattggtaaatccacaacaagttgtaacatttcaattgcattagcaaaacgtggcaaaaaagtattacaaattggttgtgatccaaaacacgatagtacttttacattaaccggttttttaattccaacaattattgatactttacaaagtaaagattatcattacgaagatgtttggccggaagatgttatttaccaaggctacgggagtgtggattgtgttgaagcaggtggcccgccagccggcgccggctgtggtgggtatgttgttggtgaaacagttaaattattaaaagaattaaatgcattttatgaatatgatgttattctgtttgatgttttaggggatgttgtatgtggtgggtttgctgcacctttaaattacgccgactattgcattattgtcacagataatggctttgatgcgttatttgccgcaaaccgtattgctgcttcagtgcgcgaaaaagcgcgcattcacccattacgtttagctgggttaattgggaatcgtacagccaaacgcgatttaatcgataaatacgttgaagcgtgcccgatgccagtcttagaggtattaccgttaattgaagacattcgtgtgtcacgcgtaaaaggtaaaacattatttgaaatggcagaacatgattcatcattacactacatttgtgacttttatttaaatattgcggatcaattattaactgaaccagaaggtgttgttccgcgcgaattagcagaccgtgaattatttactctattatcagatttctatttaaacgctgggactcctagccctagtggatctgagttcggctcaggcgcccttagcggaacgagcggcgaaacagctcccggtaatatgggtcagcacatgagtaacgcagtaaaaacaaacgaacaggaaatgaatttctttcttgtgtaa

SEQ ID NO: 18 - protein, 318 aa, Chlamydomonas reinhardtii
MKLAVYGKGGIGKSTTSCNISIALAKRGKKVLQIGCDPKHDSTFTLTGFLIPTIIDTLQSKDYHYEDVWPEDVIYQGYGSVDCVEAGGPPAGAGCGGYVVGETVKLLKELNAFYEYDVILFDVLGDVVCGGFAAPLNYADYCIIVTDNGFDALFAANRIAASVREKARIHPLRLAGLIGNRTAKRDLIDKYVEACPMPVLEVLPLIEDIRVSRVKGKTLFEMAEHDSSLHYICDFYLNIADQLLTEPEGVVPRELADRELFTLLSDFYLNAGTPSPSGSEFGSGALSGTSGETAPGNMGQHMSNAVKTNEQEMNFFLV

SEQ ID NO: 19 - DNA, 3762 bp, Chlamydomonas reinhardtii
atgcggattgtgctggtcagcggcttcgagagctttaacgtgggcctgtacaaggatgcggcggagctgctgaagcgctccatgcccaacgtcacactccaggtgttctccgaccgcgacctggcctccgacgccacccgctcccggctggaggcggctctggggcgcgccgacatcttcttcggatcactgctgttcgactacgaccaggtggagtggctacgggcccggctggagcgggtgcctgtgcggctagtgtttgagtcggcgttggagctcatgagctgcaacaaggtggggtcgttcatgatgggcggcggcggtcccggcggcggcccgcccggcaaggcgcccggcccgccgcccgcggtgaagaaggttctctccatgtttggaagcggtcgcgaggaggacaagatgggcggctcctccaatgtggtggccatgttcagttacctggtggagaccctgatggagccaacgggtgggttatttggtagttggtggttgtgttatggttggccgtttcggttgggtgatctgggctggtatctacaacccccctcaaccctcacgcctccaggctacgtgccgccgcctgtggtggagactcccgcactgggctgcctccacccctccgcgcccggccgctacttcgagtcccccgccgagtacatgaagtggtacgccagggagggcccgctgcgcggcacgggcgccccggtggttggcgtgctgctgtaccgcaagcatgtgatcaccgaccagccgtacatcccgcagctggtcagccagctggaggcggaggggctgctgcccgtgcccatcttcatcaacggcgtggaggcgcacaccgtggttcgcgacctgctgacctccgtgcacgagcaggatctgcttgcacgcggcgagacgggcgccatcagccccaccctgaagcgggacgcggtcaaggtggacgcggtggtgagcaccattggcttcccgctggtgggcggccccgccggcaccatggagggcgggcggcaggcggaggtggccaaggccatcctgggcgccaaggacgtgccgtacacggtggcggcgccgctgcttattcaggacatggagagctggagcagggacggcgtggcgggtctccagagtgtggtgctgtactcgctgccggagctggacggcgcagtggacacggtgccactgggggggctggtgggggacgacatctacctggtgccggagcgggtgaagaagctggcggggcggctcaagtcgtggcgtacgacacgcactaagcatgcctctgtttgtgacgtccagcccctcccccccccgtctcccctctccaccctccctctcccttcctctcccttcctctcactctccaccctcttccccctccgcccaaacataacgaggcgggggctgctgggcgcaagcgggccctggagtacccgctgcgacctagctagtccaactccacccatcccccaatgccgcaatagctttccggagatgagcacacacacacacacacacacacacacacacacacacacacacacacacacacacacacgccacccacgcacacacacacacacacacgctccccccgctcgccacacccccatcccaccccacccgcaggagctgctgacgtaccccgcggactggggcccggccgagtggggcccgctgccctacctgcccgaccccgacgtgctggttcgccgcatggaggcgcagtggggcgagctgcgagcctaccgcggcctcaacacctcggcgcgcggcatgttccaggagtacggggctgacgtggtcctgcacttcggcatgcacggcaccgtggagtggttgcctggggcgccgctggggaacaacggcctcagctggagcgacgtgctgctcggcgagctgccaaacgtgtacgtgtacgctgccaacaacccctccgagtccatcgtggcaaagcggcgcggctacggcaccatcgtcagccacaacgtgccgccgtacgggcgggcgggtctgtacaagcagctttccagcctcaaggagacgcttcaggagtaccgcgaggccgcgcaggccgcacgtgcccgagcaggagccagcagcagcagcggcagtagcagcagtagcagtagcagcggcagtggcagtagcagcagcagtgtggagctgcgggcggcgttggcaccggtgttcgacgcctacactgaccgcctgtatgcctacctgcagctgctggaggggcggctgttcagcgaggggctacacgtactgggagcgccgccggcgccgccgcaggtgggtggttttcccgcgagcttccaacggtaccgtaaactgcccaactgcccaacttctccccaaacacaggaggctgtcaagatccggaacctgctcatgcagaacacgcaggagctggacgggctgctcaagggcctgggtgggcgttacgtgcttcccgaggcgggcggcgacctgctgcgggacgggtcgggcgtgctgcccaccggccgcaacatccacgcactggacccctaccgcatgccctcccccgccgccatggcccgtggggcggcggtggcggcggccattcttgagcagcaccgggcggctaacagcggggcgtggcccgagacctgcgccgtcaacctgtgggggctggactccatcaagagcaagggcgagagtgtgggggtggtgctggcgctggtgggggcggtgccggtgcgcgagggtacgggccgcgtcgcgcgcttccaactggtgccgctgtcagagttgggccggccgcgtgtggacgtgctttgtaacatgagcggcatcttccgcgactccttccagaacgtggtggagctgctcgacgacctgtttgcaagggccgccgccgccgctgacgagccagatgacatgaacttcatcgccaaacacgcccgagccatggagaagcagggcctgtccgccacctcggcccgcctgttctccaacccggctggcgactacgggtcgatggtcaacgagcgagtggggcagggcagctgggccaacggcgacgagctgggtgacacgtgggcggcccgcaacgccttcagctacggccgaggcaaggagcgaggcacggcgcggcccgaggtgctgcaggcgctgctcaagaccacggaccggatcgtgcagcagatcgacagtgtggagtacggcctgacagacatccaggagtactacgccaacacgggcgccctcaagagagccgccgaggtggccaaaggcgacccgggccccggtggccggcggccgcgcgtggggtgttccattgtggaggcctttggcggcgcgggcgcgggcgcgggcggcgccggtggagcgggcgtgccgccgcctcgcgagctggaggaggtgctgcgcctggagtaccgctcgaagctgctcaaccccaagtgggcccgggccatggcggcgcagggcagcggcggcgcctacgagatcagtcagcgcatgacggcgttggtgggctggggcgccaccaccgatttcagggagggctgggtgtgggacccaggcgccatggacacgtatgtgggcgatgaggagatggccagcaagctcaagaagaacaacccgcaggcctttgccaacgtgctgcggcgcatgctggaggcggcgggccgcggcatgtggagccccaacaaggaccagctggcacagctcaagtcgctgtacagcgagatggacgaccagctggagggggtgacg

SEQ ID NO: 20 - protein, 1254 aa, Chlamydomonas reinhardtii
MRIVLVSGFESFNVGLYKDAAELLKRSMPNVTLQVFSDRDLASDATRSRLEAALGRADIFFGSLLFDYDQVEWLRARLERVPVRLVFESALELMSCNKVGSFMMGGGGPGGGPPGKAPGPPPAVKKVLSMFGSGREEDKMGGSSNVVAMFSYLVETLMEPTGGLFGSWWLCYGWPFRLGDLGWYLQPPSTLTPPGYVPPPVVETPALGCLHPSAPGRYFESPAEYMKWYAREGPLRGTGAPVVGVLLYRKHVITDQPYIPQLVSQLEAEGLLPVPIFINGVEAHTVVRDLLTSVHEQDLLARGETGAISPTLKRDAVKVDAVVSTIGFPLVGGPAGTMEGGRQAEVAKAILGAKDVPYTVAAPLLIQDMESWSRDGVAGLQSVVLYSLPELDGAVDTVPLGGLVGDDIYLVPERVKKLAGRLKSWRTTRTKHASVCDVQPLPPPSPLSTLPLPSSPFLSLSTLFPLRPNITRRGLLGASGPWSTRCDLASPTPPIPQCRNSFPEMSTHTHTHTHTHTHTHTHTHTRHPRTHTHTHAPPARHTPIPPHPQELLTYPADWGPAEWGPLPYLPDPDVLVRRMEAQWGELRAYRGLNTSARGMFQEYGADVVLHFGMHGTVEWLPGAPLGNNGLSWSDVLLGELPNVYVYAANNPSESIVAKRRGYGTIVSHNVPPYGRAGLYKQLSSLKETLQEYREAAQAARARAGASSSSGSSSSSSSSGSGSSSSSVELRAALAPVFDAYTDRLYAYLQLLEGRLFSEGLHVLGAPPAPPQVGGFPASFQRYRKLPNCPTSPQTQEAVKIRNLLMQNTQELDGLLKGLGGRYVLPEAGGDLLRDGSGVLPTGRNIHALDPYRMPSPAAMARGAAVAAAILEQHRAANSGAWPETCAVNLWGLDSIKSKGESVGVVLALVGAVPVREGTGRVARFQLVPLSELGRPRVDVLCNMSGIFRDSFQNVVELLDDLFARAAAAADEPDDMNFIAKHARAMEKQGLSATSARLFSNPAGDYGSMVNERVGQGSWANGDELGDTWAARNAFSYGRGKERGTARPEVLQALLKTTDRIVQQIDSVEYGLTDIQEYYANTGALKRAAEVAKGDPGPGGRRPRVGCSIVEAFGGAGAGAGGAGGAGVPPPRELEEVLRLEYRSKLLNPKWARAMAAQGSGGAYEISQRMTALVGWGATTDFREGWVWDPGAMDTYVGDEEMASKLKKNNPQAFANVLRRMLEAAGRGMWSPNKDQLAQLKSLYSEMDDQLEGVT

SEQ ID NO: 21 - DNA, 1254 bp, Chlamydomonas reinhardtii
atggccctgaacatgcgtgtttcctcttccaaggtcgctgccaagcagcagggccgcatctccgcggtgccggttgtgtcgagcaaggtggcctcctccgcccgcgtggcccccttccagggcgctcccgtggccgcgcagcgcgctgctctgctggtgcgcgccgctgccgctactgaggtcaaggctgctgagggccgcactgagaaggagctgggccaggcccgccccatcttccccttcaccgccatcgtgggccaggatgagatgaagctggcgctgattctgaacgtgatcgaccccaagatcggtggtgtcatgatcatgggcgaccgtggcactggcaagtccaccaccattcgtgccctggcggatctgctgcccgagatgcaggtggttgccaacgacccctttaactcggaccccaccgaccccgagctgatgagcgaggaggtgcgcaaccgcgtcaaggccggcgagcagctgcccgtgtcttccaagaagattcccatggtggacctgcccctgggcgccactgaggaccgcgtgtgcggcaccatcgacatcgagaaggcgctgaccgagggtgtcaaggcgttcgagcccggcctgctggccaaggccaaccgcggcatcctgtacgtggatgaggtcaacctgctggacgaccacctggtcgatgtgctgctggactcggccgcctccggctggaacaccgtggagcgcgagggtatctccatcagccaccccgcccgcttcatcctggtcggctcgggcaaccccgaggagggtgagctgcgcccccagctgctggatcgcttcggcatgcacgcccagatcggcaccgtcaaggacccccgcctgcgtgtgcagatcgtgtcgcagcgctcgaccttcgacgagaaccccgccgccttccgcaaggactacgaggccggccagatggcgctgacccagcgcatcgtggacgcgcgcaagctgctgaagcagggcgaggtcaactacgacttccgcgtcaagatcagccagatctgctcggacctgaacgtggacggcatccgcggcgacatcgtgaccaaccgcgccgccaaggccctggccgccttcgagggccgcaccgaggtgacccccgaggacatctaccgtgtcattcccctgtgcctgcgccaccgcctccggaaagaccccctggctgagatcgacgacggtgaccgcgtgcgtgagatcttcaagcaggtgttcggcatggagtaa

SEQ ID NO: 22 - protein, 417 aa, Chlamydomonas reinhardtii
MALNMRVSSSKVAAKQQGRISAVPVVSSKVASSARVAPFQGAPVAAQRAALLVRAAAATEVKAAEGRTEKELGQARPIFPFTAIVGQDEMKLALILNVIDPKIGGVMIMGDRGTGKSTTIRALADLLPEMQVVANDPFNSDPTDPELMSEEVRNRVKAGEQLPVSSKKIPMVDLPLGATEDRVCGTIDIEKALTEGVKAFEPGLLAKANRGILYVDEVNLLDDHLVDVLLDSAASGWNTVEREGISISHPARFILVGSGNPEEGELRPQLLDRFGMHAQIGTVKDPRLRVQIVSQRSTFDENPAAFRKDYEAGQMALTQRIVDARKLLKQGEVNYDFRVKISQICSDLNVDGIRGDIVTNRAAKALAAFEGRTEVTPEDIYRVIPLCLRHRLRKDPLAEIDDGDRVREIFKQVFGME

SEQ ID NO: 23 - DNA, 1278 bp, Chlamydomonas reinhardtii
atgcagagtctccagggtcagcgcgcgttcactgcggtgcgccagggtcgggcgggtcccctgcggactcgcctggtcgtgcgctcgtctgttgccttgccatccacgaaagccgcgaagaagccgaacttcccgttcgtcaagattcagggccaggaggagatgaagcttgcactgctgctgaacgtggtcgaccccaacatcggcggagtgcttattatgggtgaccgcggcactgccaagtcggtcgcggtccgcgccctggtggatatgcttcccgacattgacgtggttgagggcgacgccttcaacagctcccccaccgaccccaagttcatgggccccgacaccctgcagcgcttccgcaacggcgagaagctgcccaccgtccgcatgcggacccccctggtggagctgcctctgggcgccaccgaggaccgcatctgcggcaccatcgacatcgagaaggcgctgacgcagggcatcaaggcctacgagcccggcctgctggccaaggccaaccgcggcatcctgtatgtggacgaggtgaacctgctggatgatggcctggttgatgtcgtgctggactcgtcggctagcggcctgaacactgtggagcgtgagggtgtgtccattgtgcaccctgcccgcttcatcatgattggctcaggcaacccccaggagggtgagctgcgcccgcagctgctggatcgcttcggcatgagcgtcaacgtggccacgctgcaggacaccaagcagcgcacgcagctggtgctggaccggcttgcgtacgaggcggaccctgacgcatttgtggactcgtgcaaggccgagcagacggcgctcacggacaagctggaggcggcccgccagcgcctgcggtccgtcaagatcagcgaggagctgcagatcctgatctcggacatttgctcgcgcctggatgtggatggcctgcgcggtgacattgtgatcaaccgcgccgccaaggcgcttgtggccttcgagggccgcaccgaggtgaccacgaatgacgtggagcgcgtcatctcgggctgcctcaaccaccgcctgcgcaaggacccgctggaccccattgacaacggcaccaaggtggccatcctgttcaagcgcatgaccgaccccgagatcatgaagcgcgaggaggaggccaagaagaagcgcgaggaggcggccgccaaggccaaggcggagggcaaggcggaccgccccacgggcgccaaggctggcgcctgggctggcttgccccctcgtcggtaa

SEQ ID NO: 24 - protein, 425 aa, Chlamydomonas reinhardtii
MQSLQGQRAFTAVRQGRAGPLRTRLVVRSSVALPSTKAAKKPNFPFVKIQGQEEMKLALLLNVVDPNIGGVLIMGDRGTAKSVAVRALVDMLPDIDVVEGDAFNSSPTDPKFMGPDTLQRFRNGEKLPTVRMRTPLVELPLGATEDRICGTIDIEKALTQGIKAYEPGLLAKANRGILYVDEVNLLDDGLVDVVLDSSASGLNTVEREGVSIVHPARFIMIGSGNPQEGELRPQLLDRFGMSVNVATLQDTKQRTQLVLDRLAYEADPDAFVDSCKAEQTALTDKLEAARQRLRSVKISEELQILISDICSRLDVDGLRGDIVINRAAKALVAFEGRTEVTTNDVERVISGCLNHRLRKDPLDPIDNGTKVAILFKRMTDPEIMKREEEAKKKREEAAAKAKAEGKADRPTGAKAGAWAGLPPRR

SEQ ID NO: 25 - DNA, 2304 bp, Chlamydomonas reinhardtii
atgaagtctctctgccatgagctcgctggccccagcgttactgggtgcggccggcgaagcctccggaaggctttcagcggtgccaagattgcgcaggtctctcgccccgctgtgcttaacagcgtgcagcgccaacagcgtctcgcctgttctgccgtggccgagctctccgctgctgagctgcgcgccatgaaggtgtctgaggaggactccaagggcttcgatgcggatgtgtcgacccgcctggcccgctcgtaccctctggcggccgtggtgggccaggacaacatcaagcaggcgctgctgctgggcgccgtggacaccgggctgggcggcatcgccatcgccggtcgccgcggtaccgccaagtccatcatggctcgcggcctgcacgctctgctgccgcccattgaggtggtggagggcagcatctgcaacgccgaccccgaggacccccgctcctgggaggctggcctggctgagaagtatgcgggcggccctgtgaagaccaagatgcgctcggcgccgtttgtgcagatccctctgggtgtgactgaggaccgcttggtgggcactgtggacattgaggcgtccatgaaggagggcaagactgtgttccagcccggcctgctggctgaggcgcaccgcggcatcctgtacgtggacgagatcaacctgctggatgacggcattgccaacctgctgctgtccatcctgtcggacggagtcaacgtggtggagcgcgagggcatctccatcagccacccctgccggccgctgctgattgccacctacaaccccgaggagggccctctgcgtgagcacctgctggaccgcatcgccattggcctcagcgccgacgtccccagcaccagcgacgagcgcgtcaaggccattgacgcagccatccgcttccaggacaagccgcaggacactattgacgacaccgcggagctcaccgacgccctgcgcacctcggtcatcctggctcgcgagtacctgaaggacgtgaccatcgcgccggagcaggtgacctacattgtggaggaggcgcgccgcggcggagtccaggggcaccgcgcggagctgtacgcggtcaagtgtgccaaggcgtgtgcggctctggagggccgtgagcgtgtgaacaaggatgacctgcgccaggccgtgcagctggtcatcctgccgcgcgccaccatcctggaccagcccccgcccgagcaggagcagcccccgccgccgcccccgccccctcccccgccgccgccgcaggaccaaatggaggacgaggaccaggaggagaaggaggacgagaaggaggaggaggagaaggagaacgaggaccaggacgagcccgagatccctcaggagttcatgtttgagtccgagggcgtcatcatggacccctccatcctcatgttcgcgcagcagcagcagcgcgcgcagggccgctccggccgcgccaagacgctcatcttcagcgacgaccgcggccgctacatcaagcccatgctgcccaagggtgacaaggtcaagcgcctggcagtggacgccacgcttcgcgccgccgcgccctaccagaagattcgccggcagcaggccatcagcgagggcaaggtgcagcgcaaggtgtacgtggacaagccagacatgcgctccaagaagctggcccgcaaggccggtgcgctggtgatttttgttgtggacgcgtccggctccatggctctgaaccgcatgagcgccgccaagggcgcctgcatgcgcctgctggctgagtcgtacaccagccgcgaccaggtgtgcctcatccccttctacggcgacaaggccgaggtgctgctgccgccctccaagtccatcgccatggcccgccgccgcctggactcgctgccctgcggcggcggctcgccccttgcgcacggcctgtccacggcggtacgtgtgggcatgcaggccagccaggcgggcgaggtgggccgcgtcatgatggtgctcatcacggacggccgcgccaacgtcagcctggccaagtccaacgaggaccccgaggcgctcaagcccgacgcgcccaagcccaccgccgactcgctgaaggacgaggtgcgcgacatggccaagaaggccgcgtccgccggcatcaacgtgcttgtcattgacacggagaacaagttcgtgagcaccggctttgcggaggagatctccaaggcagcgcagggcaagtactactacctgcccaacgccagcgacgccgccatcgcggcggccgcgtccggcgccatggccgcggccaagggcggctactag

SEQ ID NO: 26 - protein, 767 aa, Chlamydomonas reinhardtii
MKSLCHELAGPSVTGCGRRSLRKAFSGAKIAQVSRPAVLNSVQRQQRLACSAVAELSAAELRAMKVSEEDSKGFDADVSTRLARSYPLAAVVGQDNIKQALLLGAVDTGLGGIAIAGRRGTAKSIMARGLHALLPPIEVVEGSICNADPEDPRSWEAGLAEKYAGGPVKTKMRSAPFVQIPLGVTEDRLVGTVDIEASMKEGKTVFQPGLLAEAHRGILYVDEINLLDDGIANLLLSILSDGVNVVEREGISISHPCRPLLIATYNPEEGPLREHLLDRIAIGLSADVPSTSDERVKAIDAAIRFQDKPQDTIDDTAELTDALRTSVILAREYLKDVTIAPEQVTYIVEEARRGGVQGHRAELYAVKCAKACAALEGRERVNKDDLRQAVQLVILPRATILDQPPPEQEQPPPPPPPPPPPPPQDQMEDEDQEEKEDEKEEEEKENEDQDEPEIPQEFMFESEGVIMDPSILMFAQQQQRAQGRSGRAKTLIFSDDRGRYIKPMLPKGDKVKRLAVDATLRAAAPYQKIRRQQAISEGKVQRKVYVDKPDMRSKKLARKAGALVIFVVDASGSMALNRMSAAKGACMRLLAESYTSRDQVCLIPFYGDKAEVLLPPSKSIAMARRRLDSLPCGGGSPLAHGLSTAVRVGMQASQAGEVGRVMMVLITDGRANVSLAKSNEDPEALKPDAPKPTADSLKDEVRDMAKKAASAGINVLVIDTENKFVSTGFAEEISKAAQGKYYYLPNASDAAIAAAASGAMAAAKGGY

SEQ ID NO: 27 - DNA, 4200 bp, Chlamydomonas reinhardtii
atgcagacttcctcgcttcttggccggcgcacggcccacccggctgcgggcgcgacgcccaagccggttgcgccctcgccccgcgtggctagcacccgccaggtcgcgtgcaatgtggcgactggaccccggccgcccatgaccaccttcaccggtggcaacaagggccctgctaagcagcaggtgtcgctggatctgcgcgacgagggcgctggcatgttcaccagcaccagcccggagatgcgccgtgtcgtccctgacgatgtgaagggtcgcgttaaggtgaaggttgtgtacgtggtgctggaggcccagtaccagtcggccatcagcgctgcggtgaagaacatcaacgccaagaactccaaggtgtgcttcgaggtggtgggctacctgctggaggagctgcgtgaccagaagaacctcgatatgctcaaggaggatgtggcctctgccaacatcttcatcggctcgctcatcttcattgaggagcttgccgagaagattgtggaggcggtgagccccctgcgcgagaagctggacgcgtgcctgatcttcccgtccatgccggcggtcatgaagctgaacaagctgggcacgttttcgatggctcagctgggccagtcgaagtcggtgttctcggagttcatcaagtctgctcgcaagaacaacgacaacttcgaggagggcttgctgaagctggtgcgcaccctgcctaaggtgctgaagtatctgccctcggacaaggcgcaggacgccaagaacttcgtgaacagcctgcagtactggctgggcggtaactcggacaacctggagaacctgctgctgaacaccgtcagcaactacgtgcccgctctgaagggcgtggacttcagcgtggctgagcccaccgcctaccccgatgtgggtatctggcaccctctggcctcgggcatgtacgaggacctgaaggagtacctgaactggtacgacacccgcaaggacatggtcttcgccaaggacgcccccgtcattggcctggtgctgcagcgctcgcacctggtgactggcgatgagggccactacagcggcgtggtcgctgagctggagagccgcggtgctaaggtcatccccgtctttgccggtggcctggacttctccgcccccgtcaagaagttcttctacgaccccctgggctctggccgcacgttcgtggacaccgttgtgtcgctgaccggcttcgcgctggtgggcggccccgcgcgccaggacgcgccgaaggccattgaggcgctgaagaacctgaacgtgccctacctggtgtcgctgccgctggtgttccagaccactgaggagtggctggacagcgagctgggcgtgcaccccgtccaggtggctctgcaggttgccctgcccgagctggatggtgccatggagcccatcgtgttcgctggccgtgactcgaacaccggcaagtcgcactcgctgcccgaccgcatcgcttcgctgtgcgctcgcgccgtgaactgggccaacctgcgcaagaagcgcaacgccgagaagaagctggccgtcaccgtgttcagcttcccccctgacaagggcaacgtcggcactgccgcctacctgaacgtgttcggctccatctaccgcgtgctgaagaacctgcagcgcgagggctacgacgtgggcgccctgccgccctcggaggaggatctgatccagtcggtgctgacccagaaggaggccaagttcaactcgaccgacctgcacatcgcctacaagatgaaggtggacgagtaccagaagctgtgcccttacgccgaggcgctggaggagaactggggcaagccccccggcaccctgaacaccaacggccaggagctgctggtgtacggccgccagtacggcaacgtcttcatcggcgtgcagcccaccttcggctacgagggcgacccgatgcgcctgctgttctcgaagtcggccagcccccaccacggcttcgccgcctactacaccttcctggagaagatcttcaaggccgacgccgtgctgcacttcggcacccacggctcgctggagttcatgcccggcaagcaggtcggcatgtcgggtgtgtgctaccccgactcgctgatcggcaccatccccaacctctactactacgccgccaacaacccgtctgaggccaccatcgccaagcgccgctcgtacgccaacaccatttcgtacctgacgccgcctgccgagaacgccggcctgtacaagggcctgaaggagctgaaggagctgatcagctcgtaccagggcatgcgtgagtctggccgcgccgagcagatctgcgccaccatcattgagaccgccaagctgtgcaacctggaccgcgacgtgaccctgcccgacgctgacgccaaggacctgaccatggacatgcgcgacagcgttgtgggccaggtgtaccgcaagctgatggagattgagtcccgcctgctgccctgcggcctgcacgtggtgggctgcccgcccaccgccgaggaggccgtggccaccctggtcaacatcgctgagctggaccgcccggacaacaacccccccatcaagggcatgcccggcatcctggcccgcgccattggtcgcgacatcgagtcgatttacagcggcaacaacaagggcgtcctggctgacgttgaccagctgcagcgcatcaccgaggcctcccgcacctgcgtgcgcgagttcgtgaaggaccgcaccggcctgaacggccgcatcggcaccaactggatcaccaacctgctcaagttcaccggcttctacgtggacccctgggtgcgcggcctgcagaacggcgagttcgccagcgccaaccgcgaggagctgatcaccctgttcaactacctggagttctgcctgacccaggtggtcaaggacaacgagctgggcgccctggtagaggcgctgaacggccagtacgtcgagcccggccccggcggtgaccccatccgcaaccccaacgtgctgcccaccggcaagaacatccacgccctggaccctcagtcgattcccactcaggccgcgctgaagagcgcccgcctggtggtggaccgcctgctggaccgcgagcgcgacaacaacggcggcaagtaccccgagaccatcgcgctggtgctgtggggcactgacaacatcaagacctacggcgagtcgctggcccaggtcatgatgatggtcggtgtcaagcccgtggccgacgccctgggccgcgtgaacaagctggaggtgatccctctggaggagctgggccgcccccgcgtggacgtggttgtcaactgctcgggtgtgttccgcgacctgttcgtgaaccagatgctgctgctggaccgcgccatcaagctggcggccgagcaggacgagcccgatgagatgaacttcgtgcgcaagcacgccaagcagcaggcggcggagctgggcctgcagagcctgcgcgacgcggccacccgtgtgttctccaacagctcgggctcctactcgtccaacgtcaacctggcggtggagaacagcagctggagcgacgagtcgcagctgcaggagatgtacctgaagcgcaagtcgtacgccttcaactcggaccgccccggcgccggtggcgagatgcagcgcgacgtgttcgagacggccatgaagaccgtggacgtgaccttccagaacctggactcgtccgagatctcgctgaccgatgtgtcgcactacttcgactccgaccccaccaagctggtggcgtcgctgcgcaacgacggccgcacccccaacgcctacatcgccgacaccaccaccgccaacgcgcaggtccgcactctgggtgagaccgtgcgcctggacgcccgcaccaagctgctcaaccccaagtggtacgagggcatgcttgcctcgggctacgagggcgtgcgcgagatccagaagcgcatgaccaacaccatgggctggtcggccacctcgggcatggtggacaactgggtgtacgacgaggccaactcgaccttcatcgaggatgcggccatggccgagcgcctgatgaacaccaaccccaacagcttccgcaagctggtggccaccttcctggaggccaacggccgcggctactgggacgccaagcccgagcagctggagcgcctgcgccagctgtacatggacgtggaggacaagattgagggcgtcgaataa

SEQ ID NO: 28 - protein, 1399 aa, Chlamydomonas reinhardtii
MQTSSLLGRRTAHPAAGATPKPVAPSPRVASTRQVACNVATGPRPPMTTFTGGNKGPAKQQVSLDLRDEGAGMFTSTSPEMRRVVPDDVKGRVKVKVVYVVLEAQYQSAISAAVKNINAKNSKVCFEVVGYLLEELRDQKNLDMLKEDVASANIFIGSLIFIEELAEKIVEAVSPLREKLDACLIFPSMPAVMKLNKLGTFSMAQLGQSKSVFSEFIKSARKNNDNFEEGLLKLVRTLPKVLKYLPSDKAQDAKNFVNSLQYWLGGNSDNLENLLLNTVSNYVPALKGVDFSVAEPTAYPDVGIWHPLASGMYEDLKEYLNWYDTRKDMVFAKDAPVIGLVLQRSHLVTGDEGHYSGVVAELESRGAKVIPVFAGGLDFSAPVKKFFYDPLGSGRTFVDTVVSLTGFALVGGPARQDAPKAIEALKNLNVPYLVSLPLVFQTTEEWLDSELGVHPVQVALQVALPELDGAMEPIVFAGRDSNTGKSHSLPDRIASLCARAVNWANLRKKRNAEKKLAVTVFSFPPDKGNVGTAAYLNVFGSIYRVLKNLQREGYDVGALPPSEEDLIQSVLTQKEAKFNSTDLHIAYKMKVDEYQKLCPYAEALEENWGKPPGTLNTNGQELLVYGRQYGNVFIGVQPTFGYEGDPMRLLFSKSASPHHGFAAYYTFLEKIFKADAVLHFGTHGSLEFMPGKQVGMSGVCYPDSLIGTIPNLYYYAANNPSEATIAKRRSYANTISYLTPPAENAGLYKGLKELKELISSYQGMRESGRAEQICATIIETAKLCNLDRDVTLPDADAKDLTMDMRDSVVGQVYRKLMEIESRLLPCGLHVVGCPPTAEEAVATLVNIAELDRPDNNPPIKGMPGILARAIGRDIESIYSGNNKGVLADVDQLQRITEASRTCVREFVKDRTGLNGRIGTNWITNLLKFTGFYVDPWVRGLQNGEFASANREELITLFNYLEFCLTQVVKDNELGALVEALNGQYVEPGPGGDPIRNPNVLPTGKNIHALDPQSIPTQAALKSARLVVDRLLDRERDNNGGKYPETIALVLWGTDNIKTYGESLAQVMMMVGVKPVADALGRVNKLEVIPLEELGRPRVDVVVNCSGVFRDLFVNQMLLLDRAIKLAAEQDEPDEMNFVRKHAKQQAAELGLQSLRDAATRVFSNSSGSYSSNVNLAVENSSWSDESQLQEMYLKRKSYAFNSDRPGAGGEMQRDVFETAMKTVDVTFQNLDSSEISLTDVSHYFDSDPTKLVASLRNDGRTPNAYIADTTTANAQVRTLGETVRLDARTKLLNPKWYEGMLASGYEGVREIQKRMTNTMGWSATSGMVDNWVYDEANSTFIEDAAMAERLMNTNPNSFRKLVATFLEANGRGYWDAKPEQLERLRQLYMDVEDKIEGVE

SEQ ID NO: 29 - DNA, 2064 bp, Chlamydomonas reinhardtii
atgaaattagcttattggatgtacgcaggtcccgctcatatcggtgtgttgcgtgttagcagctcttttaaaaatgtacatgccattatgcatgctcctttaggagatgattattttaatgtaatgcgttccatgttagaacgtgaacgtgattttacaccagtaacagccagtattgtagatcgtcatgttttagcaagaggatcgcaagaaaaagtggttgaaaatattacgcgaaaaaataaagaagaaactcctgatttaattttattaactcctacttgtacgtcaagcattttacaagaagatttacacaattttgttgaatcggcattagctaaaccagtacaaatagatgaacatgcagaccataaagtaactcaacaaagtgcactttcaagtgtatcccctttactaccgcttgaagaaaatacattaatagtaagtgaactagataagaagcttagcccgtctagcaagttgcatattaatatgcccaatatttgtattcccgaaggagaaggggaaggggagcagactaaaaattcaatttttgttaaatctgcaactttaacaaatttgtcagaagaggaactattaaatcaagaacatcataccaaaacaagaaatcactctgacgttattttagctgatgtaaaccattatcgtgtaaatgaattacaagctgcagatcgtactcttgaacaaattgtacgttattatatttctcaagcacaaaaacaaaattgtttaaacattactaaaacagccaaaccatctgtaaatattattggtatttttactttgggttttcataatcaacatgattgtcgtgaattaaaacgtttatttaatgatttaggtattcaaatcaatgaaatcatacctgaaggcggaaatgtacacaacttaaaaaaattaccccaagcttggtttaattttgtgccctaccgtgaaattggcttaatgactgctatgtatttaaaatccgagtttaatatgccttacgtcgcaattactcctatgggattaattgatacggctgcttgtattcgttcaatttgtaaaatcattacaactcaattattaaatcagacggctacagtgcaggagccatcaaaatttatttacccgaaggcgacgtcattagaacaaaccaatattctcgaaacctctcaaaaagaaactattcttaaagacaatccagatagcggaaataccctttctacaactgtagaagaaattgaaactttatttaataaatatatcgatcaacaaactcgttttgtttcccaagcagcctggttttcacgttctattgactgtcaaaatttaacaggtaaaaaagccgtagttttcggagatgctacacattcagctgccatgacaaaattattagcacgtgaaatgggtattaaggtttcatgcgctggaacttattgcaaacacgatgcggattggtttagagagcaagttagtgggttttgtgatcaagttttaattaccgatgatcacacacaagtaggggatatgattgcacaattagaacctgcagccatttttgggacacaaatggaacgtcacgttggtaaacgtttagatattccatgtggtgttatatctgctcctgtgcatattcaaaactttccgttaggttatcgaccttttttaggttatgaaggtacaaatcaaatagctgatttagtgtataattcatttaatcttggaatggaagaccatttattacaaatttttggaggtcatgattcagaaaacaattcgtcaattgcaacgcatttgaatacaaataacgcaataaatttagcgccaggatatttacctgagggagaaggcagtagtagaacttcaaatgtagtgtctacaatttctagtgaaaaaaaagccattgtatggtctccagaaggtttagcagaattaaataaagtcccaggatttgttcgaggaaaagttaaacgtaatacggaaaaatatgctttacaaaaaaattgttcgatgattactgtagaagttatgtatgcagcaaaagaagctttgtcggcttaa

SEQ ID NO: 30 - protein, 687 aa, Chlamydomonas reinhardtii
MKLAYWMYAGPAHIGVLRVSSSFKNVHAIMHAPLGDDYFNVMRSMLERERDFTPVTASIVDRHVLARGSQEKVVENITRKNKEETPDLILLTPTCTSSILQEDLHNFVESALAKPVQIDEHADHKVTQQSALSSVSPLLPLEENTLIVSELDKKLSPSSKLHINMPNICIPEGEGEGEQTKNSIFVKSATLTNLSEEELLNQEHHTKTRNHSDVILADVNHYRVNELQAADRTLEQIVRYYISQAQKQNCLNITKTAKPSVNIIGIFTLGFHNQHDCRELKRLFNDLGIQINEIIPEGGNVHNLKKLPQAWFNFVPYREIGLMTAMYLKSEFNMPYVAITPMGLIDTAACIRSICKIITTQLLNQTATVQEPSKFIYPKATSLEQTNILETSQKETILKDNPDSGNTLSTTVEEIETLFNKYIDQQTRFVSQAAWFSRSIDCQNLTGKKAVVFGDATHSAAMTKLLAREMGIKVSCAGTYCKHDADWFREQVSGFCDQVLITDDHTQVGDMIAQLEPAAIFGTQMERHVGKRLDIPCGVISAPVHIQNFPLGYRPFLGYEGTNQIADLVYNSFNLGMEDHLLQIFGGHDSENNSSIATHLNTNNAINLAPGYLPEGEGSSRTSNVVSTISSEKKAIVWSPEGLAELNKVPGFVRGKVKRNTEKYALQKNCSMITVEVMYAAKEALSA

SEQ ID NO: 31 - DNA, 882 bp, Chlamydomonas reinhardtii
atgaaattagctgtttacggaaaaggtggtattggaaaatcaacgacaagttgtaatatttcgattgctttacgaaaacgtggtaaaaaagtgttacaaattggttgtgatcctaaacatgatagtacttttacattgacagggtttttaattccaaccattattgatacattaagttctaaagattatcattatgaagatatttggcccgaagatgttatttacggaggttatgggggtgtagattgtgttgaagctggaggaccacctgccggtgcggggtgtggtggttatgttgtaggtgaaacggtaaaacttttaaaagagttaaatgcttttttcgaatacgatgttattttatttgatgttttaggtgatgttgtttgtggtggctttgctgctccattaaactacgctgattattgtattattgtaactgataatggttttgatgctttatttgctgcaaatcgtattgcagcttcagttcgtgaaaaagcacgtacacatccattgcgtttagcgggtttaatcggaaatcgtacatcaaaacgtgatttaattgataaatatgtagaagcttgtcctatgccagtattagaagttttaccattaattgaagaaattcgtatttcacgtgttaaaggcaaaactttatttgaaatgtcaaataaaaataatatgacttcggctcatatggatggctctaaaggtgacaattctacagtaggagtgtcagaaactccatcggaagattatatttgtaatttttatttaaatattgctgatcaattattaacagaaccagaaggagttattccacgtgaattagcagataaagaactttttactcttttatcagatttctatcttaaaatttaa

SEQ ID NO: 32 - protein, 293 aa, Chlamydomonas reinhardtii
MKLAVYGKGGIGKSTTSCNISIALRKRGKKVLQIGCDPKHDSTFTLTGFLIPTIIDTLSSKDYHYEDIWPEDVIYGGYGGVDCVEAGGPPAGAGCGGYVVGETVKLLKELNAFFEYDVILFDVLGDVVCGGFAAPLNYADYCIIVTDNGFDALFAANRIAASVREKARTHPLRLAGLIGNRTSKRDLIDKYVEACPMPVLEVLPLIEEIRISRVKGKTLFEMSNKNNMTSAHMDGSKGDNSTVGVSETPSEDYICNFYLNIADQLLTEPEGVIPRELADKELFTLLSDFYLKI

SEQ ID NO: 33 - DNA, 1410 bp, Chlamydomonas reinhardtii
atgttagatggtgccacaacgattttaaatttaaatagtttttttgaatgtgaaactggcaattatcatactttttgcccgattagctgtgtagcttggttatatcaaaaaatcgaagatagcttttttttagtaattgggacaaaaacatgtggttattttttacaaaatgcccttggagttatgatttttgccgaacctaggtatgctatggcagaattagaagaaagtgatatttcagcacaattaaacgattataaagaattaaaacgtttatgtttacaaattaaacaagatagaaatcccagcgttattgtttggattggaacttgtacaactgaaattatcaaaatggatttagaagggatggctccacgtttagaaactgaaatcggcatacccattgttgtagcacgtgctaatggtttagattatgcttttacacaaggtgaagacacagttttatcagcaatggccttagcatccttaaaaaaagatgttccttttttagtaggtaatactgggttaacaaacaaccagcttctccttgaaaaatcaacttcttcagttaatgggacagacggaaaggaattacttaaaaaatctcttgtattatttggttccgtaccaagtacagttactacacaattaactttagaattaaaaaaagaaggtattaatgtatctggatggcttccatctgctaattataaagatttacctacttttaataaagatacacttgtatgtggtataaatccttttttaagtcgaacagctaccacgttaatgcgtcgtagtaagtgcacattaatttgtgcaccctttccaataggccccgatggcacaagagtttggattgaaaaaatttgtggtgcttttggcattaatcctagtcttaatccaattactggtaatactaatttatatgatcgtgaacaaaaaattttcaacgggctagaagattatttaaaattattacgtggaaaatctgtattttttatgggtgataatttattagaaatttctttagcacgttttttaacacgttgtggtatgattgtttatgaaatcggaattccatatttagataaacgatttcaagcagcagaattagctttattagaacaaacttgtaaagaaatgaatgtaccaatgccgcgcattgtagaaaaaccagataattattatcaaattcgacgtatacgtgaattaaaacctgatttaacgattactggaatggcacatgcaaatccattagaagctcgaggtattacaacaaaatggtcagttgaatttacttttgctcaaattcatggatttactaatacacgtgaaattttagaattagtaacacagcctcttagacgcaatctaatgtcaaatcaatctgtaaatgctatttcttaa

SEQ ID NO: 34 - protein, 469 aa, Chlamydomonas reinhardtii
MLDGATTILNLNSFFECETGNYHTFCPISCVAWLYQKIEDSFFLVIGTKTCGYFLQNALGVMIFAEPRYAMAELEESDISAQLNDYKELKRLCLQIKQDRNPSVIVWIGTCTTEIIKMDLEGMAPRLETEIGIPIVVARANGLDYAFTQGEDTVLSAMALASLKKDVPFLVGNTGLTNNQLLLEKSTSSVNGTDGKELLKKSLVLFGSVPSTVTTQLTLELKKEGINVSGWLPSANYKDLPTFNKDTLVCGINPFLSRTATTLMRRSKCTLICAPFPIGPDGTRVWIEKICGAFGINPSLNPITGNTNLYDREQKIFNGLEDYLKLLRGKSVFFMGDNLLEISLARFLTRCGMIVYEIGIPYLDKRFQAAELALLEQTCKEMNVPMPRIVEKPDNYYQIRRIRELKPDLTITGMAHANPLEARGITTKWSVEFTFAQIHGFTNTREILELVTQPLRRNLMSNQSVNAIS

SEQ ID NO: 35 - DNA, 1050 bp, Chlamydomonas reinhardtii
atgcagcagtgcgttggccgctccgtccgcgctccgtccagcagggcggtcgcgcccaaggtcgctggcgctcgtgtcagccgccgcgtgtgccgcgtctatgcctccgctgttgctaccaagacggtgaagattggcacgcgcggctcgcccctggctctggcccaggcttacatgactcgcgacctgctgaagaagagcttccctgagctgagcgaggagggtgctctggagatcgtgatcatcaagaccaccggtgacaaaatcctgaaccagcccctggctgacatcggtggcaagggtctgtttaccaaggagatcgatgatgctctgctgagcggcaagattgacatcgccgtgcactccatgaaggacgtgcccacctacctgcccgagggcaccatcctgccctgcaacctgccccgcgaggatgtgcgcgatgtgttcatctcgcctgtcgccaaggacctgagcgagctgcccgccggcgccattgtgggctcggcctcgctgcgccgtcaggcccagatcctggccaagtacccccacctcaaggtggagaacttccgcggcaacgtgcagacccgcctgcgcaagctgaacgagggcgcctgctccgccaccctgctggctctggccggtctgaagcgcctggacatgactgagcacatcaccaagaccctcagcattgacgagatgctgcccgccgtgagccagggcgccattggcattgcctgccgcaccgacgacggcgccagccgcaacctgctggccgccctgaaccacgaggagacccgcatcgccgtggtgtgcgagcgcgccttcctgaccgccctggacggctcttgccgcacccccattgccggctacgcgcacaagggcgccgacggcatgctgcacttcagcggcctggtggccaccccggacggcaagcagatcatgcgcgctagccgcgtggtgcccttcacggaggcggatgccgtcaagtgcggcgaggaggccggcaaggagctcaaggccaacggccccaaggagctgttcatgtactaa

SEQ ID NO: 36 - protein, 349 aa, Chlamydomonas reinhardtii
MQQCVGRSVRAPSSRAVAPKVAGARVSRRVCRVYASAVATKTVKIGTRGSPLALAQAYMTRDLLKKSFPELSEEGALEIVIIKTTGDKILNQPLADIGGKGLFTKEIDDALLSGKIDIAVHSMKDVPTYLPEGTILPCNLPREDVRDVFISPVAKDLSELPAGAIVGSASLRRQAQILAKYPHLKVENFRGNVQTRLRKLNEGACSATLLALAGLKRLDMTEHITKTLSIDEMLPAVSQGAIGIACRTDDGASRNLLAALNHEETRIAVVCERAFLTALDGSCRTPIAGYAHKGADGMLHFSGLVATPDGKQIMRASRVVPFTEADAVKCGEEAGKELKANGPKELFMY

SEQ ID NO: 37 - DNA, 1143 bp, Chlamydomonas reinhardtii
atgcgatcgtatctgctcaaggctcaagtggcctcatgtcagttttcgcgcacgtcgaaggtctggagactggcgccgggttctgacagacgacggtgtcggggcctcactcggacaccgcactgcgcggcccccaccagcgagcccgccccgccatccagcagcggcaagagcgggcaacgaccactcgtgatagccacgcggccatctaagcttgcaaaggagcagacgcggcaggtgcagcagctgctgctggcggcggcgcagctcaaggacgagcagctgcagctgagcaccctggaactggcgtctaggggcgacacgactcagggtgtgtcgctgcgcagtctgggctcgggcgcattcaccgaggagctggaccaggctgtgctgtcgggcgctgccgacatgtcggtgcacagcctgaaggactgccccgccgccctggcgcccgggctgctgctggccgcctgcctgccgcgggccgacccccgggacgtcctcatcgcgcccgaggccacctcgctgggcgagctggtgccgggcagccgtgtgggcaccagcagcagccgccgcgcggcgcagatcaagcactccttcccccacctgcaggttgtgcagctgcgcggcaatgtggactcgcggctggggcgcatccgcagccgcgacatcggcgccacagtgctggcggcggcgggcctcaagcggctgggtgtgatgaactcggacgagggtgacactaccgctacgggcgccgtgggggtggtgtgcagggcagacgatgagtgggtggtcggcctgctggacgccatctcgcaccgcggcacggccctggaggtggcggcggagcgggcgtgcctggcagcgctgctgggcggcggcggcgcgtgccagcgttcagcgttcccggacattgcgtgggcctgccacacgcggcacgaccccgacagcaacacaatggacctggattgcctggtggcggacctggagggcaaggagctcttcaggtacacggagttctaccggccggtcattgacgaggtggacgcggtgtcgctggggtcgctgtacggcagcctgctgcgcatgatggcgccaccaggcgcggccccctgttggcagctaccttcctcgcggcattag

SEQ ID NO: 38 - protein, 380 aa, Chlamydomonas reinhardtii
MRSYLLKAQVASCQFSRTSKVWRLAPGSDRRRCRGLTRTPHCAAPTSEPAPPSSSGKSGQRPLVIATRPSKLAKEQTRQVQQLLLAAAQLKDEQLQLSTLELASRGDTTQGVSLRSLGSGAFTEELDQAVLSGAADMSVHSLKDCPAALAPGLLLAACLPRADPRDVLIAPEATSLGELVPGSRVGTSSSRRAAQIKHSFPHLQVVQLRGNVDSRLGRIRSRDIGATVLAAAGLKRLGVMNSDEGDTTATGAVGVVCRADDEWVVGLLDAISHRGTALEVAAERACLAALLGGGGACQRSAFPDIAWACHTRHDPDSNTMDLDCLVADLEGKELFRYTEFYRPVIDEVDAVSLGSLYGSLLRMMAPPGAAPCWQLPSSRH

SEQ ID NO: 39 - DNA, 1692 bp, Chlamydomonas reinhardtii
atgatgttgacccagactcctgggaccgccacggcttctagccggcggtcgcagatccgctcggctgcgcacgtctccgccaaggtcgcgcctcggcccacgccattctcggtcgcgagccccgcgaccgctgcgagccccgcgaccgcggcggcccgccgcacactccaccgcactgctgcggcggccactggtgctcccacggcgtccggagccggcgtcgccaagacgctcgacaatgtgtatgacgtgatcgtggtcggtggaggtctctcgggcctggtgaccggccaggccctggcggctcagcacaaaattcagaacttccttgttacggaggctcgcgagcgcgtcggcggcaacattacgtccatgtcgggcgatggctacgtgtgggaggagggcccgaacagcttccagcccaacgatagcatgctgcagattgcggtggactctggctgcgagaaggaccttgtgttcggtgaccccacggctccccgcttcgtgtggtgggagggcaagctgcgccccgtgccctcgggcctggacgccttcaccttcgacctcatgtccatccccggcaagatccgcgccgggctgggcgccatcggcctcatcaacggagccatgccctccttcgaggagagtgtggagcagttcatccgccgcaacctgggcgatgaggtgttcttccgcctgatcgagcccttctgctccggcgtgtacgcgggcgacccctccaagctgtccatgaaggcggccttcaacaggatctggattctggagaagaacggcggcagcctggtgggaggtgccatcaagctgttccaggaacgccagtccaacccggccccgccgcgggacccgcgcctgccgcccaagcccaagggccagacggtgggctcgttccgcaagggcctgaagatgctgccggacgccattgagcgcaacatccccgacaagatccgcgtgaactggaagctggtgtctctgggccgcgaggcggacgggcggtacgggctggtgtacgacacgcccgagggccgtgtcaaggtgtttgcccgcgccgtggctctgaccgcgcccagctacgtggtggcggacctggtcaaggagcaggcgcccgccgccgccgaggccctgggctccttcgactacccgccggtgggcgccgtgacgctgtcgtacccgctgagcgccgtgcgggaggagcgcaaggcctcggacgggtccgtgccgggcttcggtcagctgcacccgcgcacgcagggcatcaccactctgggcaccatctacagctccagcctgttccccggccgcgcgcccgagggccacatgctgctgctcaactacatcggcggcaccaccaaccgcggcatcgtcaaccagaccaccgagcagctggtggagcaggtggacaaggacctgcgcaacatggtcatcaagcccgacgcgcccaagccccgtgtggtgggcgtgcgcgtgtggccgcgcgccatcccgcagttcaacctgggccacctggagcagctggacaaggcgcgcaaggcgctggacgcggcggggctgcagggcgtgcacctggggggcaactacgtcagcggtgtggccctgggcaaggtggtggagcacggctacgagtccgcagccaacctggccaagagcgtgtccaaggccgcagtcaaggcctaa

SEQ ID NO: 40 - protein, 563 aa, Chlamydomonas reinhardtii
MMLTQTPGTATASSRRSQIRSAAHVSAKVAPRPTPFSVASPATAASPATAAARRTLHRTAAAATGAPTASGAGVAKTLDNVYDVIVVGGGLSGLVTGQALAAQHKIQNFLVTEARERVGGNITSMSGDGYVWEEGPNSFQPNDSMLQIAVDSGCEKDLVFGDPTAPRFVWWEGKLRPVPSGLDAFTFDLMSIPGKIRAGLGAIGLINGAMPSFEESVEQFIRRNLGDEVFFRLIEPFCSGVYAGDPSKLSMKAAFNRIWILEKNGGSLVGGAIKLFQERQSNPAPPRDPRLPPKPKGQTVGSFRKGLKMLPDAIERNIPDKIRVNWKLVSLGREADGRYGLVYDTPEGRVKVFARAVALTAPSYVVADLVKEQAPAAAEALGSFDYPPVGAVTLSYPLSAVREERKASDGSVPGFGQLHPRTQGITTLGTIYSSSLFPGRAPEGHMLLLNYIGGTTNRGIVNQTTEQLVEQVDKDLRNMVIKPDAPKPRVVGVRVWPRAIPQFNLGHLEQLDKARKALDAAGLQGVHLGGNYVSGVALGKVVEHGYESAANLAKSVSKAAVKA

SEQ ID NO: 41 - DNA, 1173 bp, Chlamydomonas reinhardtii
atgcagaccaaggctttcacctctgcgcgcccccagcgggccgctgcgctcaaggcgcagcgcacctcgtcggtgaccgtgcgcgcgaccgcggcccccgccgtggcctctgcccccgccgcctcgggctctgcctctgaccccctgatgctgcgcgccatccgcggcgacaaggtggagcgcccgcccgtgtggatgatgcgccaggccggccgctaccagaaggtgtaccaggacctgtgcaagaagcaccccacgttccgtgagcgctcggagcgcgtggacctggcggtggagatctctctgcagccgtggcacgcgttcaagcccgacggcgtcatcctgttcagcgacattctgacccccctgcccggcatgaacatccccttcgacatggcgcccggccccatcatcatggaccccatccgcaccatggcgcaagtggagaaggtgacgaagctggacgctgaggccgcctgccccttcgtgggcgagtcgctgcgccagctgcgcacctacatcggcaaccaggccgcggtcctgggcttcgtgggcgcccccttcaccctggccacctacattgtggagggcggcagctccaagaacttcgcgcacatcaagaagatggctttctccacccccgagatcctgcacgccctgctggacaagctggctgacaacgtggccgactacgtccgctaccaggccgacgccggcgcccaggtggtgcagatcttcgactcgtgggccagcgagctgcagccccaggacttcgacgtgttctccggcccctacatcaagaaggtgatcgacagcgtgcgcaagacccaccccgacctgcccatcatcctctacatcagcggctctggcggcctgctggagcgcatggcctcttgctcgcccgacatcatctcgctggaccagtcggtggacttcaccgacggcgtcaagcgctgcggcaccaacttcgccttccagggcaacatggaccccggcgtcctgttcggctccaaggacttcatcgagaagcgcgtcatggacaccatcaaggctgcccgcgacgccgacgtgcgccacgtgatgaacctgggccacggcgtgctgcccggcacccccgaggaccacgtgggccactacttccacgtcgcccgcaccgcccacgagcgcatgtaa

SEQ ID NO: 42 - protein, 390 aa, Chlamydomonas reinhardtii
MQTKAFTSARPQRAAALKAQRTSSVTVRATAAPAVASAPAASGSASDPLMLRAIRGDKVERPPVWMMRQAGRYQKVYQDLCKKHPTFRERSERVDLAVEISLQPWHAFKPDGVILFSDILTPLPGMNIPFDMAPGPIIMDPIRTMAQVEKVTKLDAEAACPFVGESLRQLRTYIGNQAAVLGFVGAPFTLATYIVEGGSSKNFAHIKKMAFSTPEILHALLDKLADNVADYVRYQADAGAQVVQIFDSWASELQPQDFDVFSGPYIKKVIDSVRKTHPDLPIILYISGSGGLLERMASCSPDIISLDQSVDFTDGVKRCGTNFAFQGNMDPGVLFGSKDFIEKRVMDTIKAARDADVRHVMNLGHGVLPGTPEDHVGHYFHVARTAHERM

SEQ ID NO: 43 - DNA, 288 bp, Chlamydomonas reinhardtii
atgtcggccctggacgccgccgccatcccctacgagctagtgccgggtgtgtcctccgctctggccgccccgctgttcgccggcgtcccgctcacacacgtcagcctgagcccctcgttcaccgtggtcagcgggcacgacgtggccggcaccgactgggcggcgttccgggggctgcccacgctggtggttctgatggcgggtcgtaacctggggcagatagcccggcggcttgtgcaggacgcggggtgggcgcccgatacacctgtaagtcaacctagtggctag

SEQ ID NO: 44 - protein, 95 aa, Chlamydomonas reinhardtii
MSALDAAAIPYELVPGVSSALAAPLFAGVPLTHVSLSPSFTVVSGHDVAGTDWAAFRGLPTLVVLMAGRNLGQIARRLVQDAGWAPDTPVSQPSG

SEQ ID NO: 45 - DNA, 204 bp, Chlamydomonas reinhardtii
ggcgtccccacaaccaggacagcctacttcttgaccttattaataagtcgctgcgtgtcgcgactgaccattttggcccggacttgcgtgcttgtgatttgtgcttcgactagatccgcgggcaccaagggacgcggacagctgatagtcaagaactagatcctctgggagcgtctggggctgtccccgctgctcgccaaggaa

SEQ ID NO: 46 - DNA, 721 bp, Chlamydomonas reinhardtii
gtgccgagtgactgaggtggcaaggtgcagtggcggcggaggcagttgtgctggggtggcaaggcggacaggcgaagctggtgggttgcgacgaggaggaggtgcacgtgcacgcgtaacataagaagaacagtgggaggacaggtagcgtgacttgactgggacgaggagcgtactgatgtgtggcgtgtgttggtatgtgagcgttacccctcccctagatagcggcggtctccactttcaggaggatgagagccatcatgaggctttgagggggcactggttcgtgtgtaggctgaggctgctgttgaagtcacaaggcagcactgcatgcgcgagtgagtgtggccggatatgcatcgagttgcaggtacactgaaatgaggtgactgcggcgtatatcgctgccagtacaggttgaagcggcgggcacggtgaatggagtactcggcctggaacgcttgcgatcagatggtcgagctcaagaagatttggttgagccgttgggtcgtgcgtcatattatggcttgcatcttcggggagcggcaagaaacggactccaatgcaggccctcgggcgagaaagattgggcgtgtccgggggtgcattctcgccgcgtggggctgcatcgaatttcgcttgagtgccccttcccggggagggggggcggtagttcaaccccatcatcgtaggggggttgtaaatgccagcccaaactaaa

SEQ ID NO: 47 - DNA, 187 bp, Chlamydomonas reinhardtii
atgaagtctctctgccatgagctcgctggccccagcgttactgggtgcggccggcgaagcctccggaaggctttcagcggtgccaagattgcgcaggtctctcgccccgctgtgcttaacagcgtgcagcgccaacagcgtctcgcctgttctgccgtggccgagctctccgctgctgagctgcgcg

SEQ ID NO: 48 - DNA, 281 bp, Chlamydomonas reinhardtii
ccatgaaggtgtctgaggaggactccaagggcttcgatgcggatgtgtcgacccgcctggcccgctcgtaccctctggcggccgtggtgggccaggacaacatcaagcaggcgctgctgctgggcgccgtggacaccgggctgggcggcatcgccatcgccggtcgccgcggtaccgccaagtccatcatggctcgcggcctgcacgctctgctgccgcccattgaggtggtggagggcagcatctgcaacgccgaccccgaggacccccgctcctgggag

SEQ ID NO: 49 - DNA, 132 bp, Chlamydomonas reinhardtii
gctggcctggctgagaagtatgcgggcggccctgtgaagaccaagatgcgctcggcgccgtttgtgcagatccctctgggtgtgactgaggaccgcttggtgggcactgtggacattgaggcgtccatgaag

SEQ ID NO: 50 - DNA, 167 bp, Chlamydomonas reinhardtii
gagggcaagactgtgttccagcccggcctgctggctgaggcgcaccgcggcatcctgtacgtggacgagatcaacctgctggatgacggcattgccaacctgctgctgtccatcctgtcggacggagtcaacgtggtggagcgcgagggcatctccatcagccaccc

SEQ ID NO: 51 - DNA, 163 bp, Chlamydomonas reinhardtii
ctgccggccgctgctgattgccacctacaaccccgaggagggccctctgcgtgagcacctgctggaccgcatcgccattggcctcagcgccgacgtccccagcaccagcgacgagcgcgtcaaggccattgacgcagccatccgcttccaggacaagccgcag

SEQ ID NO: 52 - DNA, 48 bp, Chlamydomonas reinhardtii
gacactattgacgacaccgcggagctcaccgacgccctgcgcacctcg

SEQ ID NO: 53 - DNA, 123 bp, Chlamydomonas reinhardtii
gtcatcctggctcgcgagtacctgaaggacgtgaccatcgcgccggagcaggtgacctacattgtggaggaggcgcgccgcggcggagtccaggggcaccgcgcggagctgtacgcggtcaag

SEQ ID NO: 54 - DNA, 171 bp, Chlamydomonas reinhardtii
tgtgccaaggcgtgtgcggctctggagggccgtgagcgtgtgaacaaggatgacctgcgccaggccgtgcagctggtcatcctgccgcgcgccaccatcctggaccagcccccgcccgagcaggagcagcccccgccgccgcccccgccccctcccccgccgccgccgcag

SEQ ID NO: 55 - DNA, 87 bp, Chlamydomonas reinhardtii
gaccaaatggaggacgaggaccaggaggagaaggaggacgagaaggaggaggaggagaaggagaacgaggaccaggacgagcccgag

SEQ ID NO: 56 - DNA, 225 bp, Chlamydomonas reinhardtii
atccctcaggagttcatgtttgagtccgagggcgtcatcatggacccctccatcctcatgttcgcgcagcagcagcagcgcgcgcagggccgctccggccgcgccaagacgctcatcttcagcgacgaccgcggccgctacatcaagcccatgctgcccaagggtgacaaggtcaagcgcctggcagtggacgccacgcttcgcgccgccgcgccctaccagaag

SEQ ID NO: 57 - DNA, 67 bp, Chlamydomonas reinhardtii
attcgccggcagcaggccatcagcgagggcaaggtgcagcgcaaggtgtacgtggacaagccagaca

SEQ ID NO: 58 - DNA, 653 bp, Chlamydomonas reinhardtii
tgcgctccaagaagctggcccgcaaggccggtgcgctggtgatttttgttgtggacgcgtccggctccatggctctgaaccgcatgagcgccgccaagggcgcctgcatgcgcctgctggctgagtcgtacaccagccgcgaccaggtgtgcctcatccccttctacggcgacaaggccgaggtgctgctgccgccctccaagtccatcgccatggcccgccgccgcctggactcgctgccctgcggcggcggctcgccccttgcgcacggcctgtccacggcggtacgtgtgggcatgcaggccagccaggcgggcgaggtgggccgcgtcatgatggtgctcatcacggacggccgcgccaacgtcagcctggccaagtccaacgaggaccccgaggcgctcaagcccgacgcgcccaagcccaccgccgactcgctgaaggacgaggtgcgcgacatggccaagaaggccgcgtccgccggcatcaacgtgcttgtcattgacacggagaacaagttcgtgagcaccggctttgcggaggagatctccaaggcagcgcagggcaagtactactacctgcccaacgccagcgacgccgccatcgcggcggccgcgtccggcgccatggccgcggccaagggcggctactag

SEQ ID NO: 59 - DNA, 379 bp, Chlamydomonas reinhardtii
gtgagcgcctactttgatatgtaccaaagataccactgataggtttaggcacggaagatctggacttggaccccgtttgcgcaagccgggcgatgcacccatttcgcggtcacgccgagcgctggggtgcaatttagcgtgcccgacaagctagaaaacagggaattaccatttgtttaattttgttgcgagagatctttgcttgtgtccaccggccgcgcgggggaacttccggtgttgcgcaaggttgcgtgcgtgcccaccatcaacacctgtgccaggtctgtgtcacccccaggttccaccaccctgcaatcttccaattgtgtctcgtttgctcgttgtctaatagtcgtcctttgctcatccctacctgcag

SEQ ID NO: 60 - DNA, 267 bp, Chlamydomonas reinhardtii
gtgaggcagggaaggtgacacaggaggttttgaaagagagacagggaggcaaagatggatggcggggcgggcagtgactttggggcggcatggagtgggattggtggagtgggattgggcaccatgtatcacagatgttggcaacacagcgcagggccttgctctgtgcttgtgttgaccgtctagtcccccgtgccctgaaccaagtctttcctcctgacacggtcctccatgtcctccttccggcattcccttcctcgtccacag

SEQ ID NO: 61 - DNA, 273 bp, Chlamydomonas reinhardtii
gtgagccagcaagggaggagaggggaacggccgggtagggcagccggagtttaaccacgccaattcaacggggagcaacggggaagaggaagggccggaagaggacggcaaaagcatttggtgggggcagcggctgtagtcagaagcgcaaaggctgccacagtgtggcccgcaccctcctcaccaccagtttggcatgatcgtttagcatgggctggaatactcaccgccagttctctcctctcccctctcctcccctgtccccgcctgcag

SEQ ID NO: 62 - DNA, 166 bp, Chlamydomonas reinhardtii
gtgagtgcgcgcgctgggtgtgtttgtgggacggcgcggcattggagcgcaggtgcgggtgctgggccgtgcacttgtccgttggttcccttggaagcttcgatacacactcttactgcacgctctttaaccgccccccccctccacctctgcccgccccgtgcag

SEQ ID NO: 63 - DNA, 275 bp, Chlamydomonas reinhardtii
gtgggtgggggaaagtgactggatgtcggtgggttttaggtatgtgcgtgtgtacgatgcggggagcagtacggaagcgggcacgagcggtgagggggcaggattgtggcgcacgctcgggccaagcccgggctcgcgacagagggtgggcttgtattcgtagtcaagcgcatcaggaagtgcagttgactggattcacctgaaacggcgctgagcgggcggctaatagaatcccgcttcctgtccgcccctccccttgcccttcaatccgtcag

SEQ ID NO: 64 - DNA, 200 bp, Chlamydomonas reinhardtii
gtgagtggcgggggccgtgcgtttgtttgttgcgtgggctggctggctggctttgttggatgagggcgctgctcaccactcatctctttgaatccccacttatccagttgcctgcatgaaaccccgcctgactcactccccaccatcctgtaccgcttttccaaacatccttgcaaccatcccgccatccccacccgcag

SEQ ID NO: 65 - DNA, 690 bp, Chlamydomonas reinhardtii
gtgaggagttggagggggaaggggcgaggggatgcgacagaagcgagggcgaggggagccggggtgggttgttgcaagtgtcgtgaattatagaatgaccccaaaagcgccggcccaacagggcctattacttgcgagtcaatccaacccctgatatagggagaatggggtagaggtcgtatcacgacagcaaggatgtacagtgggccttggggttgggaggtacagggaaaaaggagaggacatggggttgggtaagcggggaataacaaatatacacccagcgtttatggaagtgggagatggaaacgggggcggacgaacaggaacaggggccggatggaggggctatgggggcatggtgggtgggggtacggcgcggggcagagcagggtcttgggtgaatgggcaagatgctgatgcttgggatgaagacactatgagcaaagaaatggttgttgacgattgccatgatcatcgcagtgggggaggcggggtggcaataccggcagtcaacagttggggtgcgatcaagattgattggagtaccagcagtggccgggatctggctgacgtgtctcgagcgagttgctggggtggcaaggagatgcaggggcagacgacgttgtgcgaccacacttacacacatttccttccccttgcgtgtgtccgtgcgccctgtgcctccag

SEQ ID NO: 66 - DNA, 123 bp, Chlamydomonas reinhardtii
gtacgtaaacgtatttgattgctcaggtggttagccttggtgtggctgctgtttgacttgtgcagctgtctttgtgtacatgttccacaaccctgtactccccatattccgcccccattccag

SEQ ID NO: 67 - DNA, 228 bp, Chlamydomonas reinhardtii
gtgagaggcggcgcggcggcttgcgggcgaaggcggggggcggggcggaggcaatgcggccgcgcatggccagcaacggaagggctggctatcaacacggcgagcgcacgatattcatataagagtgccatcgtgcaatgctgaatacttgcgccaaccggatctcgctgctccgcttccaccggactgctttctcatctctccccttcaccctgtgtgtatccacag

SEQ ID NO: 68 - DNA, 146 bp, Chlamydomonas reinhardtii
gtgagtgcccgaggtggtgggtggtgaattggggcacgagggtatgtgggcctaagggagctgaatggggcatgttttcttctgagcatcacggtcagagcttgacctgtcctccccgctgtacccccgtgcacggtccgacacag

SEQ ID NO: 69 - DNA, 168 bp, Chlamydomonas reinhardtii
gtgagtacagcgcatcccggcgcaatcattgggcctagttactgctgcaggactcgtgtgctcttaagggctggcagctgtcagaagctctactcctcgcactgaccactgtgcctttctctccttcctctctccctccccgcacccctcctcccacttcctcaacag

SEQ ID NO: 70 - DNA, 143 bp, Chlamydomonas reinhardtii
gcagacttccataaagctcttgtaacgctgtaccaactagtaagcggtacaattcgcctgagcccgagcaacgcgacctttcttgctctgtggatctctgataatctaaccagaccaaaaccttttcactaatctaggcaaca

SEQ ID NO: 71 - DNA, 381 bp, Chlamydomonas reinhardtii
aaaaggctggtgtaggcctgtcgggtcgtgttaaaggttgctgcgtgaacgtgtaagtgtgacagtgtgccggtatgtgtgtgtatacatgtgttgcggtgtgcttttgtggcggtacatggtgatgactgagcgggtgggacagagcacggttaactgacgagggcagtccgtgcgagacggacgtttttgtagccgaggtgcaaggactgatgacgggctaagctgctggagacttggagttgagagtgcaggtggatcgacggtttctctaaggagtatgaataggcaggagggctggagacatttggggtgcaaggaggcggtagtatgggagatgtccatgggcggattttggcctctgtaacttcttaacgccca

SEQ ID NO: 72 - DNA, 252 bp, Chlamydomonas reinhardtii
atgcagagtctccagggtcagcgcgcgttcactgcggtgcgccagggtcgggcgggtcccctgcggactcgcctggtcgtgcgctcgtctgttgccttgccatccacgaaagccgcgaagaagccgaacttcccgttcgtcaagattcagggccaggaggagatgaagcttgcactgctgctgaacgtggtcgaccccaacatcggcggagtgcttattatgggtgaccgcggcactgccaagtcggtcgcg

SEQ ID NO: 73 - DNA, 156 bp, Chlamydomonas reinhardtii
gtccgcgccctggtggatatgcttcccgacattgacgtggttgagggcgacgccttcaacagctcccccaccgaccccaagttcatgggccccgacaccctgcagcgcttccgcaacggcgagaagctgcccaccgtccgcatgcggacccccctg

SEQ ID NO: 74 - DNA, 102 bp, Chlamydomonas reinhardtii
gtggagctgcctctgggcgccaccgaggaccgcatctgcggcaccatcgacatcgagaaggcgctgacgcagggcatcaaggcctacgagcccggcctgctg

SEQ ID NO: 75 - DNA, 60 bp, Chlamydomonas reinhardtii
gccaaggccaaccgcggcatcctgtatgtggacgaggtgaacctgctggatgatggcctg

SEQ ID NO: 76 - DNA, 111 bp, Chlamydomonas reinhardtii
gttgatgtcgtgctggactcgtcggctagcggcctgaacactgtggagcgtgagggtgtgtccattgtgcaccctgcccgcttcatcatgattggctcaggcaacccccag

SEQ ID NO: 77 - DNA, 101 bp, Chlamydomonas reinhardtii
gagggtgagctgcgcccgcagctgctggatcgcttcggcatgagcgtcaacgtggccacgctgcaggacaccaagcagcgcacgcagctggtgctggaccg

SEQ ID NO: 78 - DNA, 127 bp, Chlamydomonas reinhardtii
gcttgcgtacgaggcggaccctgacgcatttgtggactcgtgcaaggccgagcagacggcgctcacggacaagctggaggcggcccgccagcgcctgcggtccgtcaagatcagcgaggagctgcag

SEQ ID NO: 79 - DNA, 158 bp, Chlamydomonas reinhardtii
atcctgatctcggacatttgctcgcgcctggatgtggatggcctgcgcggtgacattgtgatcaaccgcgccgccaaggcgcttgtggccttcgagggccgcaccgaggtgaccacgaatgacgtggagcgcgtcatctcgggctgcctcaaccaccg

SEQ ID NO: 80 - DNA, 211 bp, Chlamydomonas reinhardtii
cctgcgcaaggacccgctggaccccattgacaacggcaccaaggtggccatcctgttcaagcgcatgaccgaccccgagatcatgaagcgcgaggaggaggccaagaagaagcgcgaggaggcggccgccaaggccaaggcggagggcaaggcggaccgccccacgggcgccaaggctggcgcctgggctggcttgccccctcgtcggtaa

SEQ ID NO: 81 - DNA, 534 bp, Chlamydomonas reinhardtii
gtaggtaacacaagcaattatggggcgaagatctaggctccgctgatccgggcgggcaatcggcatcgtcggtgcaaccgtggggcgtctgtgcaccctttgctggtgccaggttgcctgactcgcctgcattcctgtaccgagccacattggctgctttgcagcgtgcatgggacgggtgtaggataagcgctatgtatgcgatagcgcgggtgcaccggcttggcatggcaaggttgcggggtgcacatgcgtgccagcgtcccctcagcatcagagtctggatctaagggctcagcggcttcctgcgcatgtgggtctttgcgtagtgctacgaagccttataattaaagctcatgtattgagtggtccgggtttggggcactagtagtgccaggaggcgcgtgccaggttgatatgagcatatcagcacccgttccttgcgaaacgcttccgttgtgctcccttccccaccacctccccgctcatacccatacatatggctatccgtcctctcattgcttgcccctacag

SEQ ID NO: 82 - DNA, 195 bp, Chlamydomonas reinhardtii
gtgagcgggcctaccttctgaagacagtcttacgtgttgcactgcagcggtgttgcgcacctctgcttttgcgtgcgccgggaagcgcggattgcggcctcacagatcaagcccggaaacgcttgttgtttccagcgggtggcacacacgcgcgcgcgcgcacagtgacaccctcacggccgcgctgccctgcag

SEQ ID NO: 83 - DNA, 235 bp, Chlamydomonas reinhardtii
gtgcgtagtgcatggggagaggggacgaggggaggagggcagggccaataaaccgaaccccaagtcatcgagacacagaacccgataatagctcccagatcgccaaggggtgaggcgggaagccaaggatgatgcgttggccgcattgcgtgttgacgtcaggcttacacagggtctgactggctgtgcttggggtttggcacgcttcttgactggccccgtacgcatgctgcag

SEQ ID NO: 84 - DNA, 212 bp, Chlamydomonas reinhardtii
gtgagtggtggtggtttctgggtcagcagaggacttctgtagtaggtaatgtgggccagggaagtgtggctaacatgccaaacacgggggcgcaccagtgcaagctgcattcgctgacgtgcacgggtgcaatgggtgcaaggcgaactgcaatcgcggtgcacagttgccagggctgcgctcacgcttgagtgtctgcacacgcactgcag

SEQ ID NO: 85 - DNA, 270 bp, Chlamydomonas reinhardtii
gtgcgtagcgtgcgcgcatgtacttgtctcccttgtcatgttgggaaaggtcggtccccagcctgcttgcaagatgcggccggtcagcagctgcggacggtcagcacctacgtgccgaggttgtgtaacatgaatggcgttggggcggccgacctgccacaagctgaactgcgaccagcaaggcagctgccagcaacgcacacccgacgtgctacacgcttgtgttttgacctcctaaacacacccgcccgctgtctgtcacgtccacag

SEQ ID NO: 86 - DNA, 199 bp, Chlamydomonas reinhardtii
gtaagcggcggcggcgcggggacacggagggacatttcgcgagcatgggttgaggagtcgggaggattcggtggctggccggagtcgggagtcggagtcgcgagtcggaagtcaagcttctggcggcttcgtgctgtcgggtgcgctcgccatgatggcgctgaccggagggcgtcacgctgtgtatgtgggcgcgcag

SEQ ID NO: 87 - DNA, 231 bp, Chlamydomonas reinhardtii
gtacggggcgtacagcgggggcggctgcacggggccagtgaccgacagggcagcacgcggctggcgaagagcgacaaagtgacagggtgaccaagaccgggtgatgccacgagaggggcgcgggagccgtgcattgggtcgaggagggaggaatgcaactttacactgatgcctctgtatacggccgccttccgagccctgcaaaccttcgctttcccccgacgcacgcag

SEQ ID NO: 88 - DNA, 279 bp, Chlamydomonas reinhardtii
gtgagcgcagcgtgcggtggatgcggtgcgcgtgcgggttgccaacttattattttgtacgtggacgcgtggctggcgatggcatgtcatggcgcgaatggatattgggcgaatggataccggtaatggtagcacggggcggcagggcctggcggtagtggggttgagggggcgaggactccagcgcgcgatacatgccatgttcagcatggccccaactgacagcgcccgctgccctgtgcgccccgctccctccgcgcacccgctcctcctacacag

SEQ ID NO: 89 - DNA, 36 bp, Chlamydomonas reinhardtii
ctagtctagagggaactagggaggggcaacagagaa

SEQ ID NO: 90 - DNA, 833 bp, Chlamydomonas reinhardtii
gcggcctccccttcatggtagcactagttggcgggttgtggttggactaggcggctagggtatatacctagtagcggcggctgcggagtggagggctggcgcccagcgcgagggcgtggcctttcctcctggacccgagagcgctccgcgaggagacggcgagtgagataggcagcagcgagcggagatcgatttgtgaacagttttgtggcgggatcccatagcggatgcagagaagaccttagagcagcttcctcggtggagtgaacgagccagagcggagggaaggcgcatgagggaactgcagggactggaactgcgggagtgcaggtccggtgctaggtccgctaaacagtgcggtctacgcctgtgtgtgaggtgtgcgtgtgtgtgtgagctgtgcggttttgttgtgcaaagtaggagtgagccgagccgcgcgtactttgtggcgtgtttggctgctggcgctgagagccaagagagggtaaacgggtttggtattttatggtgcggggtgaaagcagccctcgcaggaatggagcgattctgcagcatgatgcacgtgtgcctgcgcgtggatggtggctgttgatatggctctgccactccggcagcaccgctacgatacctagcggtgcctggagtggtctctctgtttggtgcgtgatgtttgggtttgccgttttgattctttgtttcgtgctgaatggctgaggcggcaagacccctcgtgccagtgtacagagcctcacggctccctcggaccccgcgtggggacgtccattcccggtggcggtgtcgcctcggcggtgtaaagcaaaaaatatttt

SEQ ID NO: 91 - DNA, 66 bp, Chlamydomonas reinhardtii
atgcagacttcctcgcttcttggccggcgcacggcccacccggctgcgggcgcgacgcccaagccg

SEQ ID NO: 92 - DNA, 36 bp, Chlamydomonas reinhardtii
gttgcgccctcgccccgcgtggctagcacccgccag

SEQ ID NO: 93 - DNA, 106 bp, Chlamydomonas reinhardtii
gtcgcgtgcaatgtggcgactggaccccggccgcccatgaccaccttcaccggtggcaacaagggccctgctaagcagcaggtgtcgctggatctgcgcgacgagg

SEQ ID NO: 94 - DNA, 161 bp, Chlamydomonas reinhardtii
gcgctggcatgttcaccagcaccagcccggagatgcgccgtgtcgtccctgacgatgtgaagggtcgcgttaaggtgaaggttgtgtacgtggtgctggaggcccagtaccagtcggccatcagcgctgcggtgaagaacatcaacgccaagaactccaag

SEQ ID NO: 95 - DNA, 135 bp, Chlamydomonas reinhardtii
gtgtgcttcgaggtggtgggctacctgctggaggagctgcgtgaccagaagaacctcgatatgctcaaggaggatgtggcctctgccaacatcttcatcggctcgctcatcttcattgaggagcttgccgagaag

SEQ ID NO: 96 - DNA, 162 bp, Chlamydomonas reinhardtii
attgtggaggcggtgagccccctgcgcgagaagctggacgcgtgcctgatcttcccgtccatgccggcggtcatgaagctgaacaagctgggcacgttttcgatggctcagctgggccagtcgaagtcggtgttctcggagttcatcaagtctgctcgcaag

SEQ ID NO: 97 - DNA, 299 bp, Chlamydomonas reinhardtii
aacaacgacaacttcgaggagggcttgctgaagctggtgcgcaccctgcctaaggtgctgaagtatctgccctcggacaaggcgcaggacgccaagaacttcgtgaacagcctgcagtactggctgggcggtaactcggacaacctggagaacctgctgctgaacaccgtcagcaactacgtgcccgctctgaagggcgtggacttcagcgtggctgagcccaccgcctaccccgatgtgggtatctggcaccctctggcctcgggcatgtacgaggacctgaaggagtacctgaactg

SEQ ID NO: 98 - DNA, 158 bp, Chlamydomonas reinhardtii
gtacgacacccgcaaggacatggtcttcgccaaggacgcccccgtcattggcctggtgctgcagcgctcgcacctggtgactggcgatgagggccactacagcggcgtggtcgctgagctggagagccgcggtgctaaggtcatccccgtctttgccg

SEQ ID NO: 99 - DNA, 260 bp, Chlamydomonas reinhardtii
gtggcctggacttctccgcccccgtcaagaagttcttctacgaccccctgggctctggccgcacgttcgtggacaccgttgtgtcgctgaccggcttcgcgctggtgggcggccccgcgcgccaggacgcgccgaaggccattgaggcgctgaagaacctgaacgtgccctacctggtgtcgctgccgctggtgttccagaccactgaggagtggctggacagcgagctgggcgtgcaccccgtccaggtggctctgcag

SEQ ID NO: 100 - DNA, 1515 bp, Chlamydomonas reinhardtii
gttgccctgcccgagctggatggtgccatggagcccatcgtgttcgctggccgtgactcgaacaccggcaagtcgcactcgctgcccgaccgcatcgcttcgctgtgcgctcgcgccgtgaactgggccaacctgcgcaagaagcgcaacgccgagaagaagctggccgtcaccgtgttcagcttcccccctgacaagggcaacgtcggcactgccgcctacctgaacgtgttcggctccatctaccgcgtgctgaagaacctgcagcgcgagggctacgacgtgggcgccctgccgccctcggaggaggatctgatccagtcggtgctgacccagaaggaggccaagttcaactcgaccgacctgcacatcgcctacaagatgaaggtggacgagtaccagaagctgtgcccttacgccgaggcgctggaggagaactggggcaagccccccggcaccctgaacaccaacggccaggagctgctggtgtacggccgccagtacggcaacgtcttcatcggcgtgcagcccaccttcggctacgagggcgacccgatgcgcctgctgttctcgaagtcggccagcccccaccacggcttcgccgcctactacaccttcctggagaagatcttcaaggccgacgccgtgctgcacttcggcacccacggctcgctggagttcatgcccggcaagcaggtcggcatgtcgggtgtgtgctaccccgactcgctgatcggcaccatccccaacctctactactacgccgccaacaacccgtctgaggccaccatcgccaagcgccgctcgtacgccaacaccatttcgtacctgacgccgcctgccgagaacgccggcctgtacaagggcctgaaggagctgaaggagctgatcagctcgtaccagggcatgcgtgagtctggccgcgccgagcagatctgcgccaccatcattgagaccgccaagctgtgcaacctggaccgcgacgtgaccctgcccgacgctgacgccaaggacctgaccatggacatgcgcgacagcgttgtgggccaggtgtaccgcaagctgatggagattgagtcccgcctgctgccctgcggcctgcacgtggtgggctgcccgcccaccgccgaggaggccgtggccaccctggtcaacatcgctgagctggaccgcccggacaacaacccccccatcaagggcatgcccggcatcctggcccgcgccattggtcgcgacatcgagtcgatttacagcggcaacaacaagggcgtcctggctgacgttgaccagctgcagcgcatcaccgaggcctcccgcacctgcgtgcgcgagttcgtgaaggaccgcaccggcctgaacggccgcatcggcaccaactggatcaccaacctgctcaagttcaccggcttctacgtggacccctgggtgcgcggcctgcagaacggcgagttcgccagcgccaaccgcgaggagctgatcaccctgttcaactacctggagttctgcctgacccag

SEQ ID NO: 101 - DNA, 713 bp, Chlamydomonas reinhardtii
gtggtcaaggacaacgagctgggcgccctggtagaggcgctgaacggccagtacgtcgagcccggccccggcggtgaccccatccgcaaccccaacgtgctgcccaccggcaagaacatccacgccctggaccctcagtcgattcccactcaggccgcgctgaagagcgcccgcctggtggtggaccgcctgctggaccgcgagcgcgacaacaacggcggcaagtaccccgagaccatcgcgctggtgctgtggggcactgacaacatcaagacctacggcgagtcgctggcccaggtcatgatgatggtcggtgtcaagcccgtggccgacgccctgggccgcgtgaacaagctggaggtgatccctctggaggagctgggccgcccccgcgtggacgtggttgtcaactgctcgggtgtgttccgcgacctgttcgtgaaccagatgctgctgctggaccgcgccatcaagctggcggccgagcaggacgagcccgatgagatgaacttcgtgcgcaagcacgccaagcagcaggcggcggagctgggcctgcagagcctgcgcgacgcggccacccgtgtgttctccaacagctcgggctcctactcgtccaacgtcaacctggcggtggagaacagcagctggagcgacgagtcgcagctgcaggagatgtacctgaagcgcaagtcgtacgccttcaactcggaccg

SEQ ID NO: 102 - DNA, 589 bp, Chlamydomonas reinhardtii
ccccggcgccggtggcgagatgcagcgcgacgtgttcgagacggccatgaagaccgtggacgtgaccttccagaacctggactcgtccgagatctcgctgaccgatgtgtcgcactacttcgactccgaccccaccaagctggtggcgtcgctgcgcaacgacggccgcacccccaacgcctacatcgccgacaccaccaccgccaacgcgcaggtccgcactctgggtgagaccgtgcgcctggacgcccgcaccaagctgctcaaccccaagtggtacgagggcatgcttgcctcgggctacgagggcgtgcgcgagatccagaagcgcatgaccaacaccatgggctggtcggccacctcgggcatggtggacaactgggtgtacgacgaggccaactcgaccttcatcgaggatgcggccatggccgagcgcctgatgaacaccaaccccaacagcttccgcaagctggtggccaccttcctggaggccaacggccgcggctactgggacgccaagcccgagcagctggagcgcctgcgccagctgtacatggacgtggaggacaagattgagggcgtcgaataa

SEQ ID NO: 103 - DNA, 79 bp, Chlamydomonas reinhardtii
gtaggtgtaattagaaggatcaaaacctagcggcctgatctgggactgacggcctcgcgcttcaatcactctgatgcag

SEQ ID NO: 104 - DNA, 83 bp, Chlamydomonas reinhardtii
gtaggcacggcagaatgctcaatgaacatgcagctacatatgtttgggatcatggctgatctctgtgcgacgggtccgcgcag

SEQ ID NO: 105 - DNA, 183 bp, Chlamydomonas reinhardtii
gtgagcagcgcggaccgagcaagcgctggcgatgcagttggatttgttgttcttgggtcaggcgctcgctcgatggccagcgcgtgtatttaatgggataagggttgagacaaagcatctcttcgggtaaaaatcttagttttcgacagcacgttgagaggcatgcaacttgctctttcgcag

SEQ ID NO: 106 - DNA, 106 bp, Chlamydomonas reinhardtii
gtgggtaaggagttgcattatcagtgtggcatggtgttgcgggcgtctggggcgctgcaacagcggcatcgtgccgaactgaccgtgccgggctacccgcgtgcag

SEQ ID NO: 107 - DNA, 231 bp, Chlamydomonas reinhardtii
gtgcgctagggttggggtctggagggtgtggattgcgcccaagtgccctgttgcgcttggcggtcgctgtcatgatgtgagggtgacgtagtgcactcaattgcctgctacgtcaccacctttgatgggctggatctgaggcaggtcagctcggttccctgctgcatccagtgtccctgtcgccctgcacgtttgacgctgttcccccttccgcactgtctcgctttgcag

SEQ ID NO: 108 - DNA, 137 bp, Chlamydomonas reinhardtii
gtgtgggcacgcgctttgggaagggaggcatacatttttggttgcggttaggctgggcgcggacttggcactcacacggtcattgcacactcatgtctcaccttcatttacggtcccttgtgccgaactacctacag

SEQ ID NO: 109 - DNA, 255 bp, Chlamydomonas reinhardtii
gtgagcagcatcagggcagagtgcatgaacggattggtggcagtggggaatggaattagacggacacgtctgggcggcaatatgttgcgctgcagtttttggggtgtagtgaactagaaaatagggaagagataggccacataacatccgaaagctcatatttttgcaaccggcgcacctatcacagcccacctgaagggttttgtagtcaacgcgtgcaactgactagatgtccccttacctgtctgatttcag

SEQ ID NO: 110 - DNA, 211 bp, Chlamydomonas reinhardtii
gtgaggcggggcggcgctgccctcggtaggggttgcagatggtgatgggtaaccgaatgcatggccaatggggagtgaaatcaggaaaggaggggtaacacaatgcagggcagcacctgaatcgtgaaggcggagttaggcagggatctgtcagttcgcctgtcacgtggatgggcgcagctgacctttgtggtgttgtggtgtggcgcag

SEQ ID NO: 111 - DNA, 192 bp, Chlamydomonas reinhardtii
gtgagctcagctgggacatgtaggggctcgggtcgccggagcatcgatgtagaattacgggaggaggggagaggggagaggattgcacgaaccgagatgagggcggtggttcgggatttcgggcaaaagctcgtgcggcaagcgttcagtgactgaagagcagtgtgcttcaactgcccctctgtccctcag

SEQ ID NO: 112 - DNA, 167 bp, Chlamydomonas reinhardtii
gtgcgaccggtgccgctgcgtggccaacagcttggtgccaccttcctgcggtgttgatttacactgtgtgcgtggatgtgttggtttttcgcaactttagtctgggctccagctctttgccttcattgatcactcgtcttacctcctgcgccatcatttgaatacag

SEQ ID NO: 113 - DNA, 154 bp, Chlamydomonas reinhardtii
gtgagccttaatgcaacacgtgtagccgttcgcatgggtggctgggtcatgctatggttggatcggcgtccgcctgcttgctactgcctgttcggtaccagcgtttactgaccccgcgtgtgccattcccaccacctaccccctcgccttgcag

SEQ ID NO: 114 - DNA, 149 bp, Chlamydomonas reinhardtii
gacagtgatatagcaataccgatataataggtttggcgggcttcaccttgtccttacccagaatgtggccctgacagtcgatttccagcccccttgccactcgctccctgatttcttcaatcaactagttgggtcgttttctcgtaagg

SEQ ID NO: 115 - DNA, 944 bp, Chlamydomonas reinhardtii
gggggcgggtggcgagtaaggcgtatggcggagcgaggagatgggctgtggcgtggccggtgttcttttgtgtgattggaaacatagacggggtgcggcacgcggcctgactgctgcgcggttggtgtggttgcggggggagcggggtcgatggggcagcgcgcacgagttggttgaaggaggagggccaggcgctgggctacacccatggtttgaggatgctagtgagtgatgtgtgcggggggcatggtgtgtaccattcagagtccagatgcacgcacggttgcgtgggagcgttccctgctgtgcatgatgatggcgccttcgatgaatcatctcttgaaggtccaaatgaaacgtctgaagtctgcagagggtggtgctggacatgccatccaggcggaagtgggcagctgtgtctgactacaaagtaggtcttgttttgcttggatagcgtttggctatgtagcgtgtattctgctcatcaatcacgccaggcgtcagggactacccatgcaagtcgggagcgtggctggctctggaaaagttgtagctgctaggtggcgttggctggggtgtcatgcatctcggcaggtaggcggtagcggtggacgacctctgcagcggagcatgtgcacaagatgtgactgcgcatgcacccgtatatgacggcgttggcgtcagttgttgagagtgaacagaggagagacgagcgaagctgccatgcccttagtggctggtgcgagaggggaagaaagagagaggaaggactttgcggcagtgccccacgccggagttggggacacggtcatcaacagggcggcggagctgggcggagtgggtgtgtgatgggacagggttcaaggcaggttggcgaggtcggagtgggtagaccagtccttcagtgcaagggcattagggcatgatgtaagggctgaagcttg

SEQ ID NO: 116 - DNA, 116 bp, Chlamydomonas reinhardtii
atggcgtcgtttggattgatgcaaaggacggtgcactgtccccagcttgtggaggagcggtgttcgccggtcgctggctgctctggtcgtggcctgccagttatccagcggcaacg

SEQ ID NO: 117 - DNA, 676 bp, Chlamydomonas reinhardtii
gcgtggcgtgtgcagtgccaccaacggtgtccagcgagggcgtgtgctgcgccggacggccgcttcgaccgacgtggtctccttcgtggaccccaatgacattagaaaacccgcagcagcagcagctggccctgcggtggataaggtcggcgttctgctgttaaaccttggcgggcccgaaaagctcgacgacgtcaagcctttcctgtataacctattcgccgacccagaaattattcgcctgccagcggcagctcagttcctgcagccgctgctcgcgacgatcatctccacgcttcgcgccccgaagagcgcggagggctatgaggccattggcggtggtagcccgttgcgtaggattacagacgagcaggcggaggcgctggcggagtctctgcgcgccaagggccaacctgcgaacgtgtacgtgggcatgcgctattggcacccctacacggaggaggcgctggagcacattaaggccgacggcgtcacgcgcctggtcatcctcccgctgtaccctcagttctccatctctaccagcggctccagccttcgactgcttgagtcgctcttcaagagcgacatcgcgctcaagtcgctgcggcacacggtcatcccgtcctggtaccagcggcggggctacgtgagcgcgatggcggacctgattgtagag

SEQ ID NO: 118 - DNA, 138 bp, Chlamydomonas reinhardtii
gagctgaagaagttccgggacgtgcccagcgtggagctgtttttctccgcgcacggcgtgcccaagtcctacgtggaggaggcgggcgacccatacaaggaggagatggaggagtgcgtgcggctcattacggacgag

SEQ ID NO: 119 - DNA, 98 bp, Chlamydomonas reinhardtii
gtcaagcggcgcggcttcgccaacacgcacacgctggcctaccagagccgcgtgggccccgcggaatggctcaagccgtacacggatgagtccatcaa

SEQ ID NO: 120 - DNA, 119 bp, Chlamydomonas reinhardtii
ggagctgggcaagcgcggcgtcaagtcgctgctggcggtgcccatcagctttgtcagcgagcacattgagacgttggaggagatcgacatggagtaccgcgagctggcggaggagagcg

SEQ ID NO: 121 - DNA, 135 bp, Chlamydomonas reinhardtii
gcatccgcaactggggccgcgtgccggcgctgaacaccaacgccgccttcatcgacgacctggcggacgcggtgatggaggcgctgccctacgtgggctgcctggccgggccgacagactcgctggtgccgctgg

SEQ ID NO: 122 - DNA, 200 bp, Chlamydomonas reinhardtii
gcgacctggagatgctgctgcaggcctacgaccgcgagcgccgcacgctgccgtcaccggtggtgatgtgggagtggggctggaccaagagcgcggagacgtggaacggccgcattgccatgattgccatcatcatcatcctggcgctggaggcagccagcggccagtccatcctcaaaaacctgttcctggcggagtag

SEQ ID NO: 123 - DNA, 66 bp, Chlamydomonas reinhardtii
gtgcgataataaatttgcatccttatgaattgctcaatgactaacgagcagcgtccgcgaccacag

SEQ ID NO: 124 - DNA, 527 bp, Chlamydomonas reinhardtii
gtgagggtggcattctgtaaagggagttgtggagttgggcagagcgagtgggtttggtcgccagggcgaggatgttgcgcgggcgttggcaggaacagggctgctagggcttgcgtggccagcgactagggtttcgactggccagcgccgccggggcgcgcttgccgaagctgcacagccccaagcgcttctgtggatcaaatggaaacttgtggcagtgtgtatgctagcgccttggcgcaagaccaattttagtggtattactgttattactgtggtagcggtgggtattcggcggcgtggttgttgttgcagccccgtgcgactaagaccgctggcaacgacagcaagccgccgcacccaggcatatacggcccaccagcaccaccgtacacaaccacgtgcctttgcactctacgcaccacagcgcgctgctgccgctcccacctcccatcccaacggcccctcttacccccacttcacaacccctcctctcacacgccctcctcttccccctcctcttccag

SEQ ID NO: 125 - DNA, 264 bp, Chlamydomonas reinhardtii
gtgggccgggcgcagcgggcgggcgggaggggcaggaggggcaggaggggaggaagggaggggaggaagggatggaaagctggcgcagcggcagcggcgggacaggtagagggcgctgccccagcggcggcaggtgggcatggtgggcgggtaggggcgacgcgtgagggactcgtcaggcatccgcatggcggcgacttgctgctcctcaccgctgacggctgcatctgctgtgtgcgtaacctggcctggctggcaccgcag

SEQ ID NO: 126 - DNA, 392 bp, Chlamydomonas reinhardtii
gtgaggcccgtgggtgggacgcggggagggacgcggggagggggagacgcgggagcgggacaagggtgaggatacggggagggaataggagaggccatggggagggatggggacacgggaggatgcacgggcctgggtggagccagggggaagtggacgacgagcccggcgggaggagggctgggtagaaggacgcgggaggtggttgggacaggtggacggggcgtgtggagcatacggcgcaagaagcgggactgagcgggttgcagggatggatgtaatcacggcaagtaagaaccccgagtggggctcagcgtgtcagcctgccttatctttcgcgcaagcgctggggttttatttcgctgtacacacgtcgcgcctttctgccgcag

SEQ ID NO: 127 - DNA, 508 bp, Chlamydomonas reinhardtii
gtgaggaggcgccggagttttgggggaaggggtgcggcgtgaagcgagatggcaggggcgaaggaaggagcggatggtggctgggtgcaagcggagaggcgacagagagtggaggttttggtggagcggttggggagaggggcgcagcagggatgcggccctggggatggcgggacagaagggagcaagtttgccaagtgaagggggggggtgctcaagaggagagggcggtggaggttaagacggccgtgctggttatgctggggttgcaaggcgcatgggcgcatggagccgggggagtttggctgtggatgggcactgcggatgggcacggcttgctactcatgtgcggtcgcggtccgcggtgtgtcagccagccaggacccatcccactgggtcttcctgcgtgcctgggactgcttgccgccacccacccattcatcaccaccactgcgcagacccaccaacaccgctgccctgaactgctctgactcttggcgctcctcag

SEQ ID NO: 128 - DNA, 686 bp, Chlamydomonas reinhardtii
gtgagtcgcgccgtcgcggttggttcgcggatgccggttggcggatgacgttcggcggttggcattgggtttgggtttgaggggttgttgggtgaggtcgggattggggtcgggattgggggtcgagcgtggggctggcgtggatgatggcgtggtctttggaaggggcttggggaggttgcgcgtgtggatgcggacagcatgggcgcgacagtgcgcatgtgcatgtgctgtgtcaaacgtctggtgcgttcagtgtgtccttgcgtgcctcccaccgtacgcagccatcccgcgcgcctggaccgtagagaccgcctacgtgtccgctagcggcctcggcctcagcctaagcgccagtagcgccagcgacacaagcaacactgtcgctaatggcagcagcggcagcagcagcagtcacgagaatgcccgcggccgggagaaagtgctcctagccgggggccgccgctagctggtttcctcagcgcgtggacggtggtgccttcatcccgaccaccccaggcgcgtccccagtcccgtcgagctcgcctgccttgtggcccgccttgaccgccctggcgccacccggtggctcgcataacgactcgctttccgttctccgcctgacgctgtccgcctgacgctctgcgcttgactctttgcgccttcctcccctcttcccccag

SEQ ID NO: 129 - DNA, 4201 bp, Artificial Sequence
  RedAlgae CHLH DNA
atgcagacttcctcgcttcttggccggcgcacggcccacccggctgcgggcgcgacgcccaagccggttgcgccctcgccccgcgtggctagcacccgccaggtcgcgtgcaatgtggcgactggaccccggccgcccatgaccaccttcaccggtggcaacaagggccctgctaagcagcaggtgtcgctggatctgcgcgacgagggcgctggcatgttcaccagcaccagcccggagatgcgccgtgtcgtccctgacgatgtgaagggtcgcgttaaggtgaaggttgtgtacgtggtgctggaggcccagtaccagtcggccatcagcgctgcggtgaagaacatcaacgccaagaactccaaggtgtgcttcgaggtggtgggctacctgctggaggagctgcgtgaccagaagaacctcgatatgctcaaggaggatgtggcctctgccaacatcttcatcggctcgctcatcttcattgaggagcttgccgagaagattgtggaggcggtgagccccctgcgcgagaagctggacgcgtgcctgatcttcccgtccatgccggcggtcatgaagctgaacaagctgggcacgttttcgatggctcagctgggccagtcgaagtcggtgttctcggagttcatcaagtctgctcgcaagaacaacgacaacttcgaggagggcttgctgaagctggtgcgcaccctgcctaaggtgctgaagtatctgccctcggacaaggcgcaggacgccaagaacttcgtgaacagcctgcagtactggctgggcggtaactcggacaacctggagaacctgctgctgaacaccgtcagcaactacgtgcccgctctgaagggcgtggacttcagcgtggctgagcccaccgcctaccccgatgtgggtatctggcaccctctggcctcgggcatgtacgaggacctgaaggagtacctgaactggtacgacacccgcaaggacatggtcttcgccaaggacgcccccgtcattggcctggtgctgcagcgctcgcacctggtgactggcgatgagggccactacagcggcgtggtcgctgagctggagagccgcggtgctaaggtcatccccgtctttgccggtggcctggacttctccgcccccgtcaagaagttcttctacgaccccctgggctctggccgcacgttcgtggacaccgttgtgtcgctgaccggcttcgcgctggtgggcggccccgcgcgccaggacgcgccgaaggccattgaggcgctgaagaacctgaacgtgccctacctggtgtcgctgccgctggtgttccagaccactgaggagtggctggacagcgagctgggcgtgcaccccgtccaggtggctctgcaggttgccctgcccgagctggatggtgccatggagcccatcgtgttcgctggccgtgactcgaacaccggcaagtcgcactcgctgcccgaccgcatcgcttcgctgtgcgctcgcgccgtgaactgggccaacctgcgcaagaagcgcaacgccgagaagaagctggccgtcaccgtgttcagcttcccccctgacaagggcaacgtcggcactgccgcctacctgaacgtgttcggctccatctaccgcgtgctgaagaacctgcagcgcgagggctacgacgtgggcgccctgtccgccctcggaggaggatctgatccagtcggtgctgacccagaaggaggccaagttcaactcgaccgacctgcacatcgcctacaagatgaaggtggacgagtaccagaagctgtgcccttacgccgaggcgctggaggagaactggggcaagccccccggcaccctgaacaccaacggccaggagctgctggtgtacggccgccagtacggcaacgtcttcatcggcgtgcagcccaccttcggctacgagggcgacccgatgcgcctgctgttctcgaagtcggccagcccccaccacggcttcgccgcctactacaccttcctggagaagatcttcaaggccgacgccgtgctgcacttcggcacccacggctcgctggagttcatgcccggcaagcaggtcggcatgtcgggtgtgtgctaccccgactcgctgatcggcaccatccccaacctctactactacgccgccaacaacccgtctgaggccaccatcgccaagcgccgctcgtacgccaacaccatttcgtacctgacgccgcctgccgagaacgccggcctgtacaagggcctgaaggagctgaaggagctgatcagctcgtaccagggcatgcgtgagtctggccgcgccgagcagatctgcgccaccatcattgagaccgccaagctgtgcaacctggaccgcgacgtgaccctgcccgacgctgacgccaaggacctgaccatggacatgcgcgacagcgttgtgggccaggtgtaccgcaagctgatggagattgagtcccgcctgctgccctgcggcctgcacgtggtgggctgcccgcccaccgccgaggaggccgtggccaccctggtcaacatcgctgagctggaccgcccggacaacaacccccccatcaagggcatgcccggcatcctggcccgcgccattggtcgcgacatcgagtcgatttacagcggcaacaacaagggcgtcctggctgacgttgaccagctgcagcgcatcaccgaggcctcccgcacctgcgtgcgcgagttcgtgaaggaccgcaccggcctgaacggccgcatcggcaccaactggatcaccaacctgctcaagttcaccggcttctacgtggacccctgggtgcgcggcctgcagaacggcgagttcgccagcgccaaccgcgaggagctgatcaccctgttcaactacctggagttctgcctgacccaggtggtcaaggacaacgagctgggcgccctggtagaggcgctgaacggccagtacgtcgagcccggccccggcggtgaccccatccgcaaccccaacgtgctgcccaccggcaagaacatccacgccctggaccctcagtcgattcccactcaggccgcgctgaagagcgcccgcctggtggtggaccgcctgctggaccgcgagcgcgacaacaacggcggcaagtaccccgagaccatcgcgctggtgctgtggggcactgacaacatcaagacctacggcgagtcgctggcccaggtcatgatgatggtcggtgtcaagcccgtggccgacgccctgggccgcgtgaacaagctggaggtgatccctctggaggagctgggccgcccccgcgtggacgtggttgtcaactgctcgggtgtgttccgcgacctgttcgtgaaccagatgctgctgctggaccgcgccatcaagctggcggccgagcaggacgagcccgatgagatgaacttcgtgcgcaagcacgccaagcagcaggcggcggagctgggcctgcagagcctgcgcgacgcggccacccgtgtgttctccaacagctcgggctcctactcgtccaacgtcaacctggcggtggagaacagcagctggagcgacgagtcgcagctgcaggagatgtacctgaagcgcaagtcgtacgccttcaactcggaccgccccggcgccggtggcgagatgcagcgcgacgtgttcgagacggccatgaagaccgtggacgtgaccttccagaacctggactcgtccgagatctcgctgaccgatgtgtcgcactacttcgactccgaccccaccaagctggtggcgtcgctgcgcaacgacggccgcacccccaacgcctacatcgccgacaccaccaccgccaacgcgcaggtccgcactctgggtgagaccgtgcgcctggacgcccgcaccaagctgctcaaccccaagtggtacgagggcatgcttgcctcgggctacgagggcgtgcgcgagatccagaagcgcatgaccaacaccatgggctggtcggccacctcgggcatggtggacaactgggtgtacgacgaggccaactcgaccttcatcgaggatgcggccatggccgagcgcctgatgaacaccaaccccaacagcttccgcaagctggtggccaccttcctggaggccaacggccgcggctactgggacgccaagcccgagcagctggagcgcctgcgccagctgtacatggacgtggaggacaagattgagggcgtcgaataa

SEQ ID NO: 130 - DNA, 263 bp, Chlamydomonas reinhardtii
tcctacagagtaaaggtctaggcgatgcgcgactgaaagactgtgaatcccggcgtcgccgtggtgggatgtgggccggtgcgctgtcgcagaggataaattacaggtatcaaacaaggttagggcgttggaaggagcggcgctagggaactgaaatcggatctgcatcggaccctcattccgcgacttgtccttcttttgcctcgccccgcagctcttgagttttgttcttgaccctttgacacgaaccaaccgatataaaa

SEQ ID NO: 131 - DNA, 843 bp, Chlamydomonas reinhardtii
gcggcaggccttcatggtcgtcgttggagcatttgcggaaaggctgatggcagcagatgcagccatgtcagttgtggctgaagttgttggctggggcgggagcgggcagcagctgctgcgagcggccgaagcagcggtgctgctttgcgtatgagaggaagaccagtgccctcgaggaggcgagtgcctgtgtgagtgtcaggacgtgtgacttcggaaactgagggcggtgagtagatgtgactggggcttgcaggaagcctactgaccctatcagaaaaggtgagcaggggtatatggtctaggagcgttgccggagcgtggctggccagtgctagccgcgcgggctctgttgctcgctggcgcgccgccgccttcacaacagatgccgtagaaatgcagcgatgtgacgaggcgtggcctattctgcaatgtgtgaggcgccaatggcgccactgacaaatggaggagtggtcaaagcttgggtacgttttgagagctgcatcgggcagcgaggatcagtgtgcggtaagaccgacggcagacggattggcaagggaataggagggacgtgggcgtgggcgcccgcgctttgtcgaggccgcatgagccggccgcttctagacccgtagcccattttgaacaagcgcccacgcgtgctcccgatgggggacatcgatcacgggaattgattaaggggcatgtgtggtgtgcaagtgagtgactggtggttccgtccctgtgaggttgtttcgttggacgtggctgccgggttgcgcgcgggctaagcgggcctgaggcagagcgctggcgtgtagccgcgagtatcgatctgtaacgtgc

SEQ ID NO: 132 - DNA, 120 bp, Chlamydomonas reinhardtii
atggccctgaacatgcgtgtttcctcttccaaggtcgctgccaagcagcagggccgcatctccgcggtgccggttgtgtcgagcaaggtggcctcctccgcccgcgtggcccccttccag

SEQ ID NO: 133 - DNA, 37 bp, Chlamydomonas reinhardtii
ggcgctcccgtggccgcgcagcgcgctgctctgctgg

SEQ ID NO: 134 - DNA, 60 bp, Chlamydomonas reinhardtii
tgcgcgccgctgccgctactgaggtcaaggctgctgagggccgcactgagaaggagctgg

SEQ ID NO: 135 - DNA, 176 bp, Chlamydomonas reinhardtii
gccaggcccgccccatcttccccttcaccgccatcgtgggccaggatgagatgaagctggcgctgattctgaacgtgatcgaccccaagatcggtggtgtcatgatcatgggcgaccgtggcactggcaagtccaccaccattcgtgccctggcggatctgctgcccgagatgcag

SEQ ID NO: 136 - DNA, 193 bp, Chlamydomonas reinhardtii
gtggttgccaacgacccctttaactcggaccccaccgaccccgagctgatgagcgaggaggtgcgcaaccgcgtcaaggccggcgagcagctgcccgtgtcttccaagaagattcccatggtggacctgcccctgggcgccactgaggaccgcgtgtgcggcaccatcgacatcgagaaggcgctgaccgagg

SEQ ID NO: 137 - DNA, 89 bp, Chlamydomonas reinhardtii
gtgtcaaggcgttcgagcccggcctgctggccaaggccaaccgcggcatcctgtacgtggatgaggtcaacctgctggacgaccacctg

SEQ ID NO: 138 - DNA, 100 bp, Chlamydomonas reinhardtii
gtcgatgtgctgctggactcggccgcctccggctggaacaccgtggagcgcgagggtatctccatcagccaccccgcccgcttcatcctggtcggctcgg

SEQ ID NO: 139 - DNA, 145 bp, Chlamydomonas reinhardtii
gcaaccccgaggagggtgagctgcgcccccagctgctggatcgcttcggcatgcacgcccagatcggcaccgtcaaggacccccgcctgcgtgtgcagatcgtgtcgcagcgctcgaccttcgacgagaaccccgccgccttccg

SEQ ID NO: 140 - DNA, 202 bp, Chlamydomonas reinhardtii
caaggactacgaggccggccagatggcgctgacccagcgcatcgtggacgcgcgcaagctgctgaagcagggcgaggtcaactacgacttccgcgtcaagatcagccagatctgctcggacctgaacgtggacggcatccgcggcgacatcgtgaccaaccgcgccgccaaggccctggccgccttcgagggccgcaccgag

SEQ ID NO: 141 - DNA, 132 bp, Chlamydomonas reinhardtii
gtgacccccgaggacatctaccgtgtcattcccctgtgcctgcgccaccgcctccggaaagaccccctggctgagatcgacgacggtgaccgcgtgcgtgagatcttcaagcaggtgttcggcatggagtaa

SEQ ID NO: 142 - DNA, 101 bp, Chlamydomonas reinhardtii
gtgtgcagttgcatctaaagaacgtccaattcatggttactgctcgtggatctaagcggttggctcaccagcgttccatggtccccgattcgtgcacgcag

SEQ ID NO: 143 - DNA, 121 bp, Chlamydomonas reinhardtii
gtgagaagccatgatacaaatataaggatttgaagcggtagatctaggacccatcgaacttgagcaccgacttgcagtccttgccttgtccggcgactgaacttctgcgcttgctttgcag

SEQ ID NO: 144 - DNA, 82 bp, Chlamydomonas reinhardtii
gtaagtgtcgcgcaaagattttctgccgggacgggtctccctcgcaacatctgaacccatggctcgtttttttgccccgcag

SEQ ID NO: 145 - DNA, 397 bp, Chlamydomonas reinhardtii
gtgcgcgcctcccccaaccccagtttggcaaatgtgtggttaagcgtcgaaagcgtgaacagaaacaggtgttgcgggggccgcggaatggctgcaatgggtgctgggggcttcggagggtctgggggcgagtttgggtatacacgggcgcgcacacttgaaggaacgctcaaggacgacagcggaggcgtggagacagcgccggcccaagcagcctgtacttgtagctgctggtcagctgaggcatcacgacttgggaccagcacccggcctcacggttgcacaaggccatcaccgcgcgccaccacccacgcctcttcaaacccatgccggcacctaccgctacccctgtgacacgctccgcacacgccgccccgcacaccccaccatgtgacag

SEQ ID NO: 146 - DNA, 156 bp, Chlamydomonas reinhardtii
gtgagagcgaggcgcggggcgtgctctgcaggctagggtgaagatcaggagagccgaagcgggcccgaacagcgcagagagaggcaagacgacacccctgccgcgttttgatcacaagattcacacccttgctctccccaacgctcccgcacatag

SEQ ID NO: 147 - DNA, 476 bp, Chlamydomonas reinhardtii
gtgagcaggggcagataggcggtcgggcggctgggcggcaggggctgtgttggctgtgttgggtgtgggctgaggctggtgggtgggctggcgggtggcagggatagcggtgaggggatggtgatggggcagaatgggcgggtgggcggacacgtggggtcgttgaagggtgtgtggggacggcaactggtatgcgatatgtcggcttggccctggcggggaaagcattcgcagaatggcgcacgaacgaggccggggagcgagcggggatgggagacgcaacctgcgctgcgaagtgcggcgcgcgctccagttgacacgttgcacgaatgtggccagtgttcgcctgagagttatgggttagaccgccagatgagccggttaagctggtggtcgcggttgatcggctgcttcccttccggttgcacgcctggcaccctaacattaccctgtccgctgctgccctttgcccacag

SEQ ID NO: 148 - DNA, 191 bp, Chlamydomonas reinhardtii
gtgagtgcagctgccgctgcggctgctgatggtgacctgtgcgaccacggggctccgcatttctggacgaagcgttgtaccatagccgtcttggtccctgatttgggccggctctggtccgaagccttgacatctacagttcaacatggccgtataacgatcctgtgcccacccacacgccaccccgccag

SEQ ID NO: 149 - DNA, 212 bp, Chlamydomonas reinhardtii
gtgagcgcgcgctctacgatacggcagacatgtacacactgcggcgcactgtagagcttgcattgcatttcaaggcctcgaaagagtagggtggtcgttctctggtggtgtccggccacaattatgcaccccggtgttggtgcagcagctgtgatgtcacaccttgcatcacccccctactgctgccgcctctcctctcttctcgcccgcag

SEQ ID NO: 150 - DNA, 211 bp, Chlamydomonas reinhardtii
gtgagcagagcaatattgcagagggaagggtggcggaagggtgataacggttggggatctagaggggcgagatggatgcacacagcgcggggttggttatgcatgcctgcatggacgcgtgcacgcacccctgatctgccggttttccaactggcgatgccgtattatgacctgcagctcaccatcctcatgcttgatttgcctcgctcag

SEQ ID NO: 151 - protein, 417 aa, Chlamydomonas reinhardtii; identical to SEQ ID NO: 22

SEQ ID NO: 152 - protein, 721 aa, Artificial Sequence
  Mutant protein sequence RedAlgaeCHLH
MQTSSLLGRRTAHPAAGATPKPVAPSPRVASTRQVACNVATGPRPPMTTFTGGNKGPAKQQVSLDLRDEGAGMFTSTSPEMRRVVPDDVKGRVKVKVVYVVLEAQYQSAISAAVKNINAKNSKVCFEVVGYLLEELRDQKNLDMLKEDVASANIFIGSLIFIEELAEKIVEAVSPLREKLDACLIFPSMPAVMKLNKLGTFSMAQLGQSKSVFSEFIKSARKNNDNFEEGLLKLVRTLPKVLKYLPSDKAQDAKNFVNSLQYWLGGNSDNLENLLLNTVSNYVPALKGVDFSVAEPTAYPDVGIWHPLASGMYEDLKEYLNWYDTRKDMVFAKDAPVIGLVLQRSHLVTGDEGHYSGVVAELESRGAKVIPVFAGGLDFSAPVKKFFYDPLGSGRTFVDTVVSLTGFALVGGPARQDAPKAIEALKNLNVPYLVSLPLVFQTTEEWLDSELGVHPVQVALQVALPELDGAMEPIVFAGRDSNTGKSHSLPDRIASLCARAVNWANLRKKRNAEKKLAVTVFSFPPDKGNVGTAAYLNVFGSIYRVLKNLQREGYDVGALSALGGGSDPVGADPEGGQVQLDRPAHRLQDEGGRVPEAVPLRRGAGGELGQAPRHPEHQRPGAAGVRPPVRQRLHRRAAHLRLRGRPDAPAVLEVGQPPPRLRRLLHLPGEDLQGRRRAALRHPRLAGVHARQAGRHVGCVLPRLADRHHPQPLLLRRQQPV

SEQ ID NO: 153 - DNA, 1254 bp, Chlamydomonas reinhardtii; identical to SEQ ID NO: 21

(19) United States
(12) Patent Application Publication
(10) Pub. No.: US 2021/0386088 A1
(43) Pub. Date: Dec. 16, 2021

(54) METHODS FOR OVERPRODUCING PROTOPORPHYRIN IX IN ALGAE AND COMPOSITIONS THEREFROM

(71) Applicant: Triton Algae Innovations, Inc., San Diego, CA (US)

(72) Inventors: Miller Tran, San Diego, CA (US); John Deaton, San Diego, CA (US); Brock Adams, San Diego, CA (US); Michael Mayfield, San Diego, CA (US); Amanda Longo, San Diego, CA (US); Oscar Gonzalez, San Diego, CA (US); Jon Hansen, San Diego, CA (US); Xun Wang, San Diego, CA (US); David Schroeder, San Diego, CA (US)

(21) Appl. No.: 17/291,599

(22) PCT Filed: Nov. 7, 2019
(86) PCT No.: PCT/US2019/060326

§ 371 Date: May 5, 2021

Related U.S. Application Data
(60) Provisional application No. 62/757,534, filed on Nov. 8, 2018.
(60) Provisional application No. 62/850,227, filed on May 20, 2019.
(60) Provisional application No. 62/865,800, filed on Jun. 24, 2019.

(51) Int. Cl. A23J 1/00; A23J 3/22; A23J 3/20; A23L 17/60; A23L 5/46; A23K 10/16; A23K 20/158; A23K 10/12; C12P 17/18; C12N 1/12; C12N 15/11; C12N 9/88

(52) U.S. Cl. CPC A23J 1/009; C12N 2310/20; A23J 3/20; A23L 17/60; A23L 5/46; A23K 10/16; A23K 20/158; A23K 10/12; C12P 17/182; C12N 1/125; C12N 15/11; C12N 9/88; C12Y 499/01001; A23V 2002/00; C12N 2500/30; C12N 2500/02; C12N 2500/34; C12N 2500/24; A23J 3/227

(57) ABSTRACT

Provided herein are compositions and processes for producing compositions from an algae that overproduces protoporphyrin IX (PPIX). Also provided are methods of growing PPIX overproducing algae, methods of isolating PPIX-containing portions from algae cultures and compositions and methods of making food products with PPIX produced by algae. Provided herein are strains and methods to select strains that overproduce PPIX. Also provided are compositions, including edible compositions that include PPIX produced from algae. Specifically, the algal strain over-producing PPIX is an engineered strain of The algal biomass may be used for preparing meat analogues wherein the PPIX compound imparts a meat-like colour and flavour.

### CROSS-REFERENCE TO RELATED APPLICATION(S)

This application claims the benefit of priority under 35 U.S.C. § 119(e) of U.S. Provisional Application No. 62/865,800, filed Jun. 24, 2019, of U.S. Provisional Application No. 62/850,227, filed May 20, 2019, and of U.S. Provisional Application No. 62/757,534, filed Nov. 8, 2018, the entire content of each of which is hereby incorporated by reference.

### SEQUENCE LISTING

The instant application contains a Sequence Listing which has been submitted electronically in ASCII format and is hereby incorporated by reference in its entirety. Said ASCII copy, created on Nov. 7, 2019, is named 20498-202380_SL.txt and is 208 kilobytes in size.

### BACKGROUND

With the advent of industrialized animal agriculture, the consumption of animal meat has continued to rise. Accounting for more than 18% of the greenhouse gases generated to date, animal agriculture is one of the leading causes of climate change. In addition to land use, animal agriculture also requires a significant amount of fresh water, a finite resource that is becoming increasingly difficult to access. It is estimated that to produce one pound of beef it takes 1,799 gallons of fresh water and to produce one pound of pork it takes 576 gallons of water. This is compared to 216 gallons of fresh water to produce 1 pound of soybean or 108 gallons to produce one pound of corn. The intensity of fresh water needed to produce animal meat is a result of the water needed to grow the plants that the animals consume and the animal's inefficiency at turning the food that it consumes into actual meat.

To address the sustainability and ethical concerns over animal meat consumption, the food industry has been aggressively trying to develop plant-based alternatives that taste, touch and smell like meat products. However, many of the current plant-based alternatives have not been able to penetrate the larger food and consumer markets. Typically these alternatives are composed of plant based materials that are extruded to generate a firm texture to improve mouth feel and subsequently mixed with various flavors and aroma forming compounds to improve the taste and smell of these products. Unfortunately, these alternatives have largely appealed to consumers who have already committed to a vegan/vegetarian lifestyle as opposed to consumers who are more accustomed to eating meat. To improve the sustainability of the food ecosystem it is imperative that products are developed that appeal to consumers who currently prefer meat. By producing the next generation of plant-based products the contribution of greenhouse gases and the demand for water resulting from the animal agriculture industry can be drastically reduced.

Recent advances have demonstrated the potential of using heme-containing proteins, purified from a host organism, to make the flavor and aroma profile of a product closer to that of meat. It is thought that the heme from heme-containing proteins are responsible for imparting a “meaty” flavor and aroma to meat products. However, the available sources of heme-containing proteins are expensive and technically intensive limiting their utility. For example, the heme binding protein, leghemoglobin, has been extracted from soy roots, but this process is proving to be expensive making its incorporation into meat alternatives less economically viable. The yeast, Pichia pastoris has been engineered to express heme binding proteins, for instance with an additional 8 enzyme pathways for producing the heme molecule. This process still requires the heme binding protein to be purified away from the expression host before it is incorporated into a finished product, a process that limits the potential positive impacts due to economic constraints. In addition to poor economics, the product is genetically modified making it less appealing to many consumers who have chosen to consume foods that are not a result of genetic engineering. Thus, a need exists for edible products incorporating heme-containing proteins as set forth herein.

### SUMMARY OF THE INVENTION

Provided herein are compositions and processes for producing such compositions that provide new sources for flavor, color, mouth feel, taste, odor, texture and nutrition for food products and food ingredients, as well as other uses such as animal feed. Provided herein are compositions and processes for producing such compositions from an algae that overproduces protoporphyrin IX. Accordingly, in an exemplary aspect, the present invention provides a composition comprising a preparation from an algae strain, wherein the strain overexpresses or accumulates protoporphyrin IX (PPIX). In some embodiments, the preparation is a biomass from the algae strain. In some embodiments, the preparation is a fractionated biomass from the algae strain. In such embodiments, it is contemplated that the fractionated biomass comprises a PPIX-enriched fraction. Further, in such embodiments, it is also contemplated that the PPIX-enriched fraction further comprises a protein-enriched fraction. In some embodiments, wherein the preparation is an extracellular fraction of the algae culture.

In some embodiments, the preparation is red or red-like in color. Alternatively and/or additionally, the preparation contains a greater amount of PPIX than of heme. Alternatively and/or additionally, the preparation contains less than about 1%, about 0.5%, about 0.1%, about 0.05%, about 0.01%, about 0.005% or about 0.001% heme. Alternatively and/or additionally, the preparation contains less than about 1%, about 0.5%, about 0.1%, about 0.05%, about 0.01%, about 0.005% or about 0.001% heme-protein.

In some embodiments, the preparation does not contain a detectable amount of a heme-protein. Alternatively and/or additionally, the preparation does not contain a detectable amount of heme. Alternatively and/or additionally, the preparation does not contain a detectable amount of protein. In some embodiments, the preparation has a protoporphyrin IX content greater than chlorophyll content.

In some embodiments, the preparation provides at least about 5%, 10%, 15%, 20%, 25%, 30%, 35%, 40%, 45%, 50%, 55%, 60%, 65%, 70%, 75%, 80%, 85%, 90%, 95%, 99% or 100% of the total protein content to the edible composition. Alternatively and/or additionally, the preparation provides vitamin A, beta carotene or a combination thereof to the composition. In such embodiments, it is preferred that the vitamin A, the beta carotene or the combination thereof is at least about 5%, 10%, 15%, 20%, 25%, 30%, 35%, 40%, 45%, 50%, 55%, 60%, 65%, 70%, 75%, 80%, 85%, 90%, 95%, 99% or 100% of the daily recommended requirement. In some embodiments, the preparation provides less than about 0.01%, 0.05%, 0.1%, 0.2%, 0.3%, 0.4%, 0.5%, 0.6%, 0.7%, 0.8%, 0.9%, 1.0%, 1.2%, 1.5%, 2%, 5% or 10% of total saturated fat present in the composition. Alternatively and/or additionally, the preparation provides at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 250 mg, 300 mg, 350 mg, 400 mg, 450 mg or 500 mg of omega-3 fatty acids to the composition.

In some embodiments, the composition has a red or red-like color derived from the preparation. Alternatively and/or additionally, the composition has a meat or meat-like flavor derived from the preparation. Alternatively and/or additionally, the composition has a meat or meat-like texture derived from the preparation.

In some embodiments, the algae is a Chlamydomonas sp. Optionally, the algae is Chlamydomonas reinhardtii. In some embodiments, the Chlamydomonas sp. is strain CC-125 deposited to the University of Minnesota Chlamydomonas collection center or a derivative thereof. In some embodiments, the algae has reduced or absence of ferrocheletase activity or expression of ferrocheletase.

Another aspect of the disclosure includes a food product comprising the composition as described herein. In some embodiments, the food product comprises a clean meat, a cultured meat, a synthetic meat, a plant-based meat or a non-animal cell-based meat. Alternatively and/or additionally, the food product is selected from the group consisting of a beef-like food product, a fish-like product, a chicken-like product, a pork-like product and a meat replica. Alternatively and/or additionally, the food product is vegan, vegetarian or gluten-free.

Another aspect of the disclosure includes an edible ingredient comprising the composition as described herein. In some embodiments, the ingredient is part of a finished product, wherein the finished product has a red or red-like color derived from the ingredient. Alternatively and/or additionally, the ingredient is part of a finished product, wherein the finished product has a meat or meat-like flavor derived from the ingredient. Alternatively and/or additionally, the ingredient is part of a finished product, wherein the finished product Hasan appearance of blood derived from the ingredient. In some embodiments, the finished product is an ingredient for a burger, a fish-alternative, a sausage, a kebab, a filet, a ground meat-like product or a meatball. In some embodiments, the edible composition is part of a finished product and wherein the finished product is an animal feed.

In some embodiments, the edible ingredient is combined with a protein source, a fat source, a carbohydrate, a starch, a thickener, a vitamin, a mineral, or any combination thereof. In some embodiments, the protein source is textured wheat protein, textured soy protein, fungal protein or algal protein. In such embodiments, it is contemplated that the finished product is free of animal proteins. In some embodiments, the fat source comprises at least one of refined coconut oil or sunflower oil. In some embodiments, the edible composition further comprises at least one of potato starch, methylcellulose, water, and a flavor, wherein the flavor is selected at least one of yeast extract, garlic powder, onion powder, and salt.

Another aspect of the disclosure includes a meat substitute comprising the composition or the edible ingredient as described herein. In some embodiments, the meat substitute further comprises (a) 0.01%-5% (by weight of the meat replica matrix) of a non-animal protoporphyrin IX, (b) a compound selected from glucose, ribose, fructose, lactose, xylose, arabinose, glucose-6-phosphate, maltose, and galactose, and any combination thereof, (c) at least 1.5 mM of a compound selected from cysteine, cystine, thiamine, methionine, and any combination thereof, and (d) one or more proteins selected from the group consisting of plant proteins, fungal proteins and algal proteins. Preferably, the meat substitute is a ground beef-like food product that contains no animal product; wherein cooking the ground beef-like food product results in the production of at least two volatile compounds which have a beef-associated aroma.

Another aspect of the disclosure includes a method of producing a protoporphyrin IX composition. The method includes steps of growing an algae population comprising an algae that is a protoporphyrin IX over-producer; and isolating a protoporphyrin IX composition from the culture. In some embodiments, the step of growing comprises culturing the algae culture in an aerobic fermentation condition. In some embodiments, the algae contains a chloroplast. In such embodiment, biosynthesis of the protoporphyrin IX occurs in the chloroplast.

In some embodiments, the algae is deficient for its ability to produce chlorophyll. Alternatively and/or additionally, the algae is deficient for the ability to produce a functional Mg-chelatase enzyme. Alternatively and/or additionally, the algae is reduced in or lacks ChlD1, ChlD2 or ChlDH. Alternatively and/or additionally, the algae is reduced in or lacks a functional light dependent protochlorophyllide. Alternatively and/or additionally, the algae is reduced in or lacks a functional light independent protochlorophyllide. Alternatively and/or additionally, the algae is reduced in or lacks ChlB, ChlL, or ChlN. Alternatively and/or additionally, the algae overexpresses one or more of glutamyl-tRNA reductase, glutamate-1-semialdehyde aminotransferase, ALA dehydratase, porphobilinogen deaminase, UPG III synthase, UPG III decarboxylase, CPG oxidase, and PPG oxidase.

In some embodiments, the algae is generated by mating and wherein the generated strain is red or red-like in color. Alternatively and/or additionally, the algae is generated by mutagenesis. In some embodiments, the algae is red or red-like in color. In some embodiments, the isolated protoporphyrin IX composition is an algae biomass. In such embodiment, it is contemplated that the algae biomass is fractionated. Alternatively and/or additionally, the algae biomass is fractionated to produce a protein-enriched fraction comprising protoporphyrin IX.

In some embodiments, the isolated protoporphyrin IX composition is isolated from extracellular media of the algae culture. Alternatively and/or additionally, the isolated protopophyrin IX composition is isolated away from algae protein. In some embodiments, the algae is deficient for carotenoids. In some embodiments, the algae is a Chlamydomonas sp. In some embodiments, the algae is a Chlamydomonas reinhardtii. In some embodiments, the Chlamydomonas sp. is strain CC-125 deposited to the University of Minnesota Chlamydomonas collection center or a derivative thereof.

In some embodiments, progeny of a protoporphyrin IX overexpressing algae strain grows faster than its parental strain following a mating with another algae. Alternatively and/or additionally, the protoporphyrin IX algae is a strain produced by mating a strain deficient for carotenoids with a strain exhibiting a red or red-like color. Alternatively and/or additionally, the protoporphyrin IX overexpressing algae is generated by mutagenesis of a first starting strain and selection of a second strain that grows faster in the dark than the first starting strain. Alternatively and/or additionally, the protoporphyrin IX algae is generated by mutagenesis of a first strain and selection of a second strain that lacks one or more carotenoids from the mutated first strain.

In some embodiments, the algae lacks a functional ferrocheletase enzyme. Alternatively and/or additionally, the algae is reduced in the amount of, or activity of, a ferrocheletase enzyme. Alternatively and/or additionally, the algae is reduced in the amount of or lacks heme as compared to a wildtype strain.

In some embodiments, the method further comprises steps of a) culturing the algae strain under a dark condition, wherein the strain does not produce or is reduced in the production of chlorophyll, and (b) collecting a portion of the algae culture that is red or red-like in color to produce the protoporphyrin IX composition. Preferably, the algae is a Chlamydomonas sp. In some embodiments, the algae is a Chlamydomonas reinhardtii. In some embodiments, the algae exhibits a red or red-like color when grown in the dark condition.

In some embodiments, the collected portion is extracellular media from the algae culture. Alternatively and/or additionally, the collected portion is a biomass or fractionated biomass from the algae culture. In some embodiments, the algae is grown in an aerobic fermentation condition. Alternatively and/or additionally, the algae is grown to a density greater than about 10 g/L, 20 g/L, 30 g/L, 40 g/L, 50 g, L, 75 g/L, 100 g/L, 125 g/L, or 150 g/L. Alternatively and/or additionally, the algae is grown with acetate as a reduced carbon source. Alternatively and/or additionally, wherein the algae is grown with sugar as a reduced carbon source. Alternatively and/or additionally, the algae culture is supplemented with iron during the culturing step. In some embodiments, the algae culture is inoculated at a density greater than about 0.1 g/L, 1.0 g/L, 5.0 g/L, 10 g/L, 20 g/L, 50 g/L, 80 g/L, or 100 g/L.

In some embodiments, the method further comprises fractionating the collected portion, wherein the fractionating removes from the collected portion substantially all or most of a component selected from the group consisting of carotenoids, starch, and protein. Alternatively and/or additionally, the method further comprises fractionating the collected portion, wherein the fractionating removes from the collected portion substantially all or most of heme, heme-binding protein or a combination thereof. Alternatively and/or additionally, the method further comprises fractionating the collected portion, wherein the fractionating produces a protein-enriched fraction.

In some embodiments, the algae lacks or is reduced in one or more of magnesium chelatase, magnesium protoporphyrinogen IX, protochlorophyllide, chlorophyllide, and chlorophyll. Alternatively and/or additionally, the algae lacks or is reduced in ferrocheletase. In some embodiments, the algae is not a transgenic strain.

Another aspect of the disclosure includes a clean meat product produced by the method described herein, and the process further comprises combining the collected portion with a clean meat manufacturing composition, wherein the collected portion provides a red or red-like color to the clean meat product. In some embodiments, the collected portion is a PPIX-enriched fraction or purified PPIX.

Another aspect of the disclosure includes an edible ingredient produced by the method described herein, and the protoporphyrin IX composition confers a meat or meat-like flavor, texture, odor or any combination thereof to the edible ingredient. In some embodiments, the edible ingredient is incorporated in a finished product selected from the group consisting of a beef-like food product, a fish-like product, a chicken-like product, a pork-like product and a meat replica. Alternatively and/or additionally, the edible ingredient is vegan, vegetarian or gluten-free. Alternatively and/or additionally, the edible ingredient is free of animal proteins. Alternatively and/or additionally, the edible ingredient does not contain any genetically modified components.

Another aspect of the disclosure includes a protoporphyrin IX-containing composition produced by the method as described herein. In some embodiments, the composition does not contain a detectable level of heme, heme-binding protein or a combination thereof.

### BRIEF DESCRIPTION OF THE DRAWINGS

FIG. 1 is a pictorial diagram showing an exemplary pathway for the production of heme in algae. This exemplary pathway can be used by wildtype algae to produce chlorophyll, but it can also be used to generate protoporphyrin IX (PPIX).

FIG. 2 is a pictorial diagram showing an exemplary fractionation of algae overexpressing PPIX, showing the rich red color of the extract.

FIG. 3 is a pictorial diagram showing burgers created with 0.01 g, 0.1 g, 1.0 g, and 5.0 g of the PPIX enriched algae.

FIG. 4 is a pictorial diagram showing ingredient mixes of the plant-based burger ingredients with no heme-enriched algae, with the addition of PPIX-enriched algae, or the ingredients with the addition of heme-enriched algae shaped into a burger before and after cooking.

FIG. 5 is a pictorial diagram showing an example of PPIX-enriched meatless “tuna”.

FIG. 6 shows a portion of sequence alignments of a wild type green algae and a red-algae with a mutation in CHLH gene (upper sequence (Seq_1) is a partial nucleic acid sequence (residues 1621-1679 of SEQ ID NO: 27) and a partial amino acid sequence (residues 451-460 of SEQ ID NO: 28) of CHLH gene of green algae, and lower sequence (Seq_2) is a partial nucleic acid sequence (residues 1621-1680 of SEQ ID NO: 129) and partial amino acid sequence (residues 451-460 of SEQ ID NO: 152) of CHLH gene of red algae has a mutation (asterisk)). As shown, the wild-type CHLH nucleic acid sequence (SEQ ID NO: 27) has an insertion of a thiamine at position 1678 resulting in a change of the wild-type CHLH amino acid sequence of SEQ ID NO: 28 of a proline to a serine at amino acid position 560.

### DETAILED DESCRIPTION

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims. As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein, “a deficiency in” or the “lack of”, or “reduction of”, one or more genes and/or enzymes include, for example, mutation or deletion of the gene sequence, a reduction in or lack in the expression from a gene (RNA and/or protein) and/or a lack of accumulation or stability of a gene product (RNA and/or protein).

As used herein, “overexpresses” and “overexpression” of an enzyme or gene include, for example, an increase in expression from a gene (RNA and/or protein) and/or an increase in accumulation or stability of a gene product (RNA and/or protein). Such overexpression can include alterations to the regulatory region(s) and/or to the gene sequence, as well as copy number, genomic position and post-translational modifications.

As used herein, the term “engineered algae” is used to refer to an algae that contains one or more genetic modifications. In some cases, an engineered algae is also a recombinantly modified organism when it incorporates heterologous nucleic acid into its genome through recombinant technology. In other cases, an engineered algae is not a recombinantly modified organism (for example when it is modified through UV, chemical or radiation mutagenesis). In some cases an algae that is not a recombinantly modified organism is referred to as non-GMO, and components from such algae can be referred to as non-GMO components.

As used herein, the term “genetic modification” is used to refer to any manipulation of an organism's genetic material in a way that does not occur under natural conditions. A genetic modification can include modifications that are made through mutagenesis (such as with UV light, X-rays, gamma irradiation and chemical exposure). A genetic modification can include gene editing. In some cases, genetic modifications can be made through recombinant technology. As used herein, “recombinantly modified organism” is used to refer to an organism that incorporates heterologous nucleic acid (e.g., recombinant nucleic acid) into its genome through recombinant technology. Methods of performing such manipulations are known to those of ordinary skill in the art and include, but are not limited to, techniques that make use of vectors for transforming cells with a nucleic acid sequence of interest. Included in the definition are various forms of gene editing in which DNA is inserted, deleted or replaced in the genome of a living organism using engineered nucleases, or “molecular scissors.” These nucleases create site-specific double-strand breaks (DSBs) at desired locations in the genome. The induced double-strand breaks are repaired through nonhomologous end-joining (NHEJ) or homologous recombination (HR), resulting in targeted mutations (i.e., edits).

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Provided herein are methods to select, grow and to incorporate algae overexpressing the molecule, protoporphyrin IX (PPIX), into food and animal feed ingredients and products. Such products can include non-genetically modified and plant-based alternative foods. Algae are known for producing many compounds that result in these aquatic organisms being various colors. These compounds include, but are not limited to, chlorophyll which makes algae green, beta-carotene which makes algae appear yellow or orange, astaxanthin which makes algae appear red or other various pigments such as phycocyanin which make algae blue. While each of these previously mentioned compounds has been added to food products, there are to date no products that incorporate an algae over-producing PPIX to impart a red color and/or a meaty taste and smell.

Provided herein are strains, methods and compositions that employ algae overproducing PPIX. In some embodiments, the algae strain when grown is red or red-like in color. As used herein, in some embodiments, red-like color can be any color with a wavelength between 590 nm to 750 nm or any mixture of the color. Alternatively and/or additionally, in some embodiments, red-like color can be defined as any color in RGB (r.g.b) having r value between 255 and 80 with g or b values between 0 and 80. In some embodiments, a preparation made from the algae culture overproducing PPIX, imparts a pink or red color when incorporated into food and other edible products. In some embodiments, a preparation made from the algae culture overproducing PPIX, imparts a “meaty” flavor, smell and/or texture when incorporated into food and other edible products.

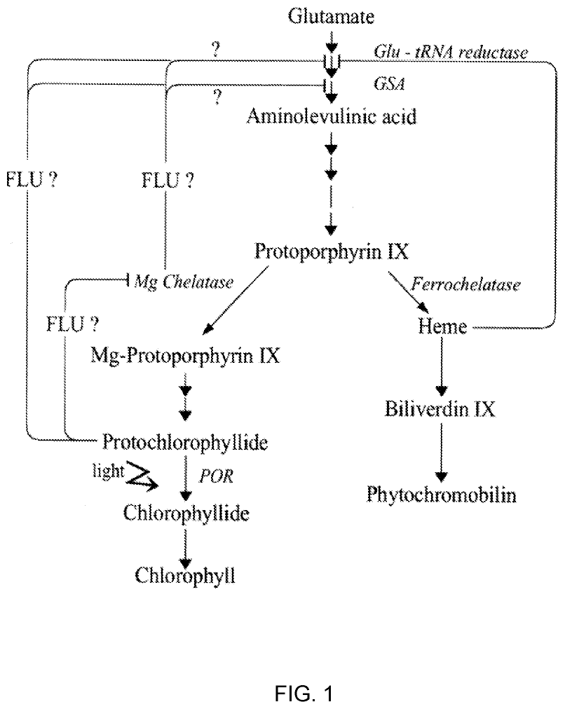

Without being bound by theory, the heme pathway is a biochemical pathway that branches from the chlorophyll biochemical pathway, as shown in FIG. 1. In short, this pathway starts with a glutamate tRNA which is converted to 5-aminolaevulinic acid (ALA) by a GlutRNA reductase and a GSA amino transferase. Next, ALA is converted to porphobilinogen by ALA dehydratase. Next, porophobilinogen is converted to hydroxymethylbilane by pophobilinogen deaminase. Next, hydroxymethylbilane is converted to uroporphyrinogen III by UPG III synthase. Next, uroporphyrinogen III is converted to coprophyrinogen by UPG III decarboxylase. Next, coprophyrinogen is converted to protoporphyringen IX by CPG oxidase. Next, protoporphyrinogen IX is converted to protoporphyrin IX by PPG oxidase. Protoporphyrin IX can be shuttled to the chlorophyll production pathway or towards heme B. Finally, protoporphyrin IX is converted to heme B by the enzyme ferrochelatase which attaches iron to protoporphyrin IX.

By reducing metabolic flux towards chlorophyll, it is possible to increase metabolic flux towards PPIX, as well as heme B. By reducing or eliminating ferrocheletase, the pathway produces PPIX but the conversion from PPIX to heme is reduced or eliminated.

In some embodiments herein, the algae strains used in the methods and compositions produced therewith are reduced in metabolic flux towards chlorophyll and increased in metabolic flux towards heme B. In some embodiments herein, an engineered algae strain contains a genetic modification in ferrocheletase, such as in one or more of the nucleotide sequence (e.g., SEQ ID NO: 7), and/or amino acid sequence (e.g., SEQ ID NO: 8), and includes genetic modifications in one or more of the regulatory regions, such as those of SEQ ID NOs: 114, 115, exons, such as those of SEQ ID NOs: 116-122, and introns, such as those of SEQ ID NOs: 123-128.

In some embodiments, the algae strain is one where chlorophyll and carotenoid synthesis is decreased. In some embodiments, the algae strain is deficient or reduced in the amount of chlorophyll. In some embodiments, the algae strain is deficient or reduced in the function, amount of, or activity of, ferrocheletase by at least 10%, by at least 20%, by at least 30%, by at least 40%, by at least 50%, compared to the wild type algae. In some embodiments, the algae strain is deficient or reduced in the amount of heme B that accumulates, and the strain is increased in accumulation of PPIX. In some embodiments, the algae strain is red or red-like in color.

In some embodiments, the algae strain is deficient for one or more enzymes in the chlorophyll biosynthesis pathway. Such deficiencies include, but are not limited to, gene deletions, mutations and other alterations that result in a lack expression of the enzyme or a deficiency in the functionality of the enzyme. In some embodiments, the algae strain is deficient in magnesium chelatase which is the first step in converting protoporphyrin IX to chlorophyll. In some embodiments, the algae strain is deficient for light dependent protochlorophyllide which converts protochlorophyllide to chlorophyllide. In some embodiments, the algae strain is deficient for a light independent protochlorophyllide reductase which converts protochlorophyllide to chlorophyllide in the dark. In some embodiments, the algae strain is deficient for one or more of ChlB, ChlL, or ChlN. In some embodiments, the algae strain is lacking or reduced in one or more of magnesium chelatase, magnesium protoporphyrin IX, protochlorophyllide, chlorophyllide, and chlorophyll.

In some embodiments, the algae strain is deficient for one or more of the magnesium chelatase subunits CHLD, CHLH and CHLI. These subunits are also referred to by the gene names, CHLD1 (alternatively written as CHlD1), corresponding to the CHLD subunit, CHLH1 (alternatively written as CHlH1), corresponding to the CHLH subunit, and CHLI1 and CHLI2, corresponding to the CHLI subunit, encoded by two genes, CHLI1 and CHLI2 (alternatively written as CHlI1 and CHlI2).

In some embodiments, the algae strain is deficient for one or more of CHLD1, CHLH1, CHLI1, CHLI2 or portions thereof (including genetic modification in one or more of intron, exon, regulatory regions, or full gene sequences, such as a genetic modification to one or more of SEQ ID NOs: 45-69, 70-88, 89-113, or 130-150. For example, one of the red algae strain has genetic modification in CHLH locus. The modification deletes a single base pair in CHLH as compared to a green strain, causing a frameshift in the CHLH open reading frame and/or generate a stop codon such that the protein is translated into a truncated form. The sequence comparison is shown in FIG. 6 (upper sequence (Seq_1) is a partial nucleic acid sequence (residues 1621-1679 of SEQ ID NO: 27) and a partial amino acid sequence (residues 451-460 of SEQ ID NO: 28) of CHLH gene of green algae, and lower sequence (Seq_2) is a partial nucleic acid sequence (residues 1621-1680 of SEQ ID NO: 129) and partial amino acid sequence (residues 451-460 of SEQ ID NO: 152) of CHLH gene of red algae has a mutation (asterisk)). The nucleic acid sequences of additional genes that may be altered in such algae strains are provided herein.

In some embodiments, an engineered algae strain for use with the methods and compositions herein includes a genetic modification in a ferrocheletase gene that decreases or is deficient in production of ferrocheletase and also has a modification in one or more enxymes leading from PPIX to chlorophyll (such as CHLD, CHLI1, CHLI2 and/or CHLH).

In some embodiments, the algae strain overexpresses one or more enzymes such that the balance of pathways favors PPIX production. In some embodiments, the algae strain overexpresses one or more of glutamyl-tRNA reductase, glutamyl-1-semialdehyde aminotransferase, ALA dehydratase, porphobilinogen deaminase, UPG III synthase, UPG III decarboxylase, CPG oxidase, and PPG oxidase. In some embodiments, the algae strain overexpresses one or more of such enzymes and is also reduced in the amount or activity of the ferrochelatase enzyme. In some embodiments, the algae strain is improved for its ability to produce ALA, a rate limiting precursor of heme B synthesis, and optionally, is reduced in the amount or activity of the ferrochelatase enzyme. In some embodiments, the algae strain is deficient for its ability to produce a functional ferrochelatase gene, the enzyme responsible for the conversion of protoporphyrin IX to heme B. In some embodiments, the algae strain is improved for its ability to produce UPG III synthase, UPG III decarboxylase, CPG oxidase, or PPG oxidase. In some embodiments, the algae strain has an increased amount of protoporphyrin IX as compared to a wildtype strain.

In some embodiments, the algae strain produces carotenoids or precursors of carotenoids. Without being bound by theory, carotenoids confer color and can have an impact on the visual appearance of a plant-based alternative. Exemplary carotenoids include, but are not limited to, gamma-carotene, beta-carotene, beta cryptoxanthin, zeaxanthin, autheraxanthin, lutein, prolycopene and lycopene.

In some embodiments, the algae strain is deficient for carotenoids or precursors of carotenoids. Deficiencies in carotenoid biosynthesis can occur due to mutations, such as mutations that impact carotenoid biosynthesis, for example, mutations in the phytoene synthase gene.

In some embodiments, the algae strain for use in the methods herein and for making PPIX-containing compositions is selected or identified based on one or more phenotypes and/or genotypes. In some embodiments, the algae strain for overproducing PPIX can be created through mating processes. In some embodiments, the algae strain for overproducing PPIX can be created through random mutagenesis, such as ultra violet mutagenesis. In some embodiments, the algae strain for overproducing PPIX can be generated through chemical mutagenesis with a compound that results in DNA alterations.

In some embodiments, modifications can be created through gene editing such as precisely engineered nuclease targeting to alter the expression of one or more components, such as by CRISPR-CAS nucleases. Such nucleases can be used to create insertions, deletions, mutations and replacements of one or more nucleotides or regions of nucleotides to modify the expression of one or more pathway enzymes in the pathway to reduce chlorophyll and/or to increase the production or accumulation of PPIX. Subsequent to the creation of the modification, the algae strain is grown and/or mated such that the nuclease and associated guide nucleic acids are removed, and the algae strain that remains does not retain the nuclease and associated editing system. In some embodiments, a nuclease such as the CRISPR-CAS nuclease is used to make a modification to a component of the chlorophyll pathway such that chlorophyll expression and/or accumulation is reduced or abrogated. In some embodiments, a nuclease such as the CRISPR-CAS nuclease is used to make a modification to a component of the chlorophyll pathway such that PPIX expression and/or accumulation is increased. In some embodiments, a nuclease such as the CRISPR-CAS nuclease is used to make a genetic modification in the gene encoding for ferrocheletase, such as a modification in one or more of SEQ ID NOs: 114-128 that reduces expression or abrogates expression of the gene, or a modification that abrogates, truncates or causes a frame shift in the gene encoding the enzyme, such as in SEQ ID NOs: 116-122, and/or a modification that alters or truncates the protein expressed such as an alteration in amino acid SEQ ID NO: 8. In some embodiments, a nuclease such as the CRISPR-CAS nuclease is used to make a modification in one or more of CHLD, CHLI1, CHLI2 or CHLH1 resulting in a PPIX-enriched algae strain. Such modifications are made by designing guide RNAs with modifications to one or more of SEQ ID NOs: 45-113, 130-150 or 153 to include one or more point mutations, insertions, deletions or combinations thereof. In some embodiments, such genetic modifications in more than one target sequence, such as those in a ferrocheletase sequence and in a another chlorophyll-pathway sequence (e.g., CHLD, CHLI1, CHLI2 or CHLH1) are combined either by concurrent or sequential rounds of nuclease engineering and/or by mating engineered algae strains containing the genetic modifications.

There are several families of engineered nucleases used in gene editing, for example, but not limited to, meganucleases, zinc finger nucleases (ZFNs), transcription activator-like effector-based nucleases (TALEN), the CRISPR-Cas system, and ARCUS. However, it should be understood that any known gene editing system utilizing engineered nucleases may be used in the methods described herein. Thus, in some embodiments, the algae strain overproducing PPIX can be genetically modified by using techniques such as a CRISPR-Cas system (e.g., CRISPR-CAS9) or by the use of zinc-finger nucleases.

CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By transfecting a cell with elements including a Cas gene and specifically designed CRISPRs, nucleic acid sequences can be cut and modified at any desired location. Methods of preparing compositions for use in genome editing using the CRISPR/Cas systems are described in detail in US Pub. No. 2016/0340661, US Pub. No. 2016/0340662, US Pub. No. 2016/0354487, US Pub. No. 2016/0355796, US Pub. No. 2016/0355797, and WO 2014/018423, which are specifically incorporated by reference herein in their entireties.

Zinc-finger nucleases (ZFNs) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms. The most common cleavage domain is the Type IIS enzyme Fok1. Fok1 catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. Proc., Natl. Acad. Sci. USA 89 (1992):4275-4279; Li et al. Proc. Natl. Acad. Sci. USA, 90:2764-2768 (1993); Kim et al. Proc. Natl. Acad. Sci. USA. 91:883-887 (1994a); Kim et al. J. Biol. Chem. 269:31,978-31,982 (1994b), all of which are incorporated herein by reference. One or more of these enzymes (or enzymatically functional fragments thereof) can be used as a source of cleavage domains.

Methods for selection of algae include, but are not limited to, genetic screening or phenotypic screening for deficiencies, mutations and changes in the chlorophyll biosynthesis pathway and/or chlorophyll accumulation, genetic screening or phenotypic screening for increased expression and/or accumulation of PPIX, PPIX biosynthesis intermediates and heme biosynthesis enzymes. In some embodiments, the algae strain for use in the methods herein and for making PPIX-containing compositions is selected or identified based on its spectral profile and/or its red or red-like color. In some embodiments, the algae for use in the methods herein and for making PPIX-containing compositions is selected or identified based on its growth rate in dark conditions. In some embodiments, the selection is based on growth rate in dark conditions and the appearance or enhancement of a red or red-like color when grown in dark conditions. In some embodiments, an algae strain is selected which is deficient in or reduced in the amount of carotenoids produced or accumulated.

In some embodiments, algae strains are mated to combine or enhance characteristics that contribute to PPIX production, PPIX accumulation, reduction in chlorophyll and/or reduction in carotenoids. In some embodiments, an algae strain that has fast growth under dark conditions (e.g., faster than a wildtype strain) is mated with an algae strain that exhibits a red or red-like color. Thus, such algae strain is not a transgenic strain. In some embodiments, an algae strain deficient for carotenoid production or accumulation is mated with an algae strain exhibiting a red or red-like color. It is contemplated that such generated algae is a protoporphyrin IX overexpressing algae strain grows faster than its parental strain following a mating with another algae.

In some embodiments, an algae strain is mutagenized and then a new strain is selected or identified that exhibits one or more characteristics of increased PPIX production, PPIX accumulation, reduction in chlorophyll and/or reduction in carotenoids. In some embodiments, an algae strain is generated by mutagenesis of a first starting strain and selection of a second strain that grows faster in the dark than the first starting strain. In some embodiments, an algae strain is generated by mutagenesis of a first starting strain and selection of a second strain that lacks one or more carotenoids.

### Algae for Use in the Compositions and Methods

In the compositions and methods provided herein for producing PPIX and PPIX-containing compositions, algae strains that have a PPIX biosynthesis pathway are employed. In some embodiments, the algae strain for overproducing PPIX is a Chlorophyta (green algae). In some embodiments, the green algae is selected from the group consisting of Chlamydomonas, Dunaliella, Haematococcus, Chlorella, and Scenedesmaceae. In some embodiments, the Chlamydomonas is a Chlamydomonas reinhardtii. In varying embodiments, the green algae can be a Chlorophycean, a Chlamydomonas, C. reinhardtii, C. reinhardtii 137c, or a psbA deficient C. reinhardtii strain. In some embodiments, the selected host is Chlamydomonas reinhardtii, such as in Rasala and Mayfield, Bioeng Bugs. (2011) 2(1):50-4; Rasala, et al., Plant Biotechnol J. (2011) May 2, PMID 21535358; Coragliotti, et al., Mol Biotechnol. (2011) 48(1):60-75; Specht, et al., Biotechnol Lett. (2010) 32(10):1373-83; Rasala, et al., Plant Biotechnol J. (2010) 8(6):719-33; Mulo, et al., Biochim Biophys Acta. (2011) May 2, PMID:21565160; and Bonente, et al., Photosynth Res. (2011) May 6, PMID:21547493; US Pub. No. 2012/0309939; US Pub. No. 2010/0129394; and Intl. Pub. No. WO 2012/170125. All of the foregoing references are incorporated herein by reference in their entirety for all purposes.

In some embodiments, the algae strain for overproducing PPIX is a single-celled algae. Illustrative and additional microalgae species of interest include without limitation, Achnanthes orientalis, Agmenellum, Amphiprora hyaline, Amphora coffeiformis, Amphora coffeiformis linea, Amphora coffeiformis punctata, Amphora coffeiformis taylori, Amphora coffeiformis tenuis, Amphora delicatissima, Amphora delicatissima capitata, Amphora sp., Anabaena, Ankistrodesmus, Ankistrodesmus falcatus, Boekelovia hooglandii, Borodinella sp., Botryococcus braunii, Botryococcus sudeticus, Carteria, Chaetoceros gracilis, Chaetoceros muelleri, Chaetoceros muelleri subsalsum, Chaetoceros sp., Chlamydomonas sp., Chlamydomonas reinhardtii, Chlorella anitrata, Chlorella Antarctica, Chlorella aureoviridis, Chlorella candida, Chlorella capsulate, Chlorella desiccate, Chlorella ellipsoidea, Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorella kessleri, Chlorella lobophora (strain SAG 37.88), Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris, Chlorella vulgaris f. tertia, Chlorella vulgaris var. autotrophica, Chlorella vulgaris var. viridis, Chlorella vulgaris var. vulgaris, Chlorella vulgaris var. vulgaris f. tertia, Chlorella vulgaris var. vulgaris f. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Ellipsoidon sp., Euglena, Franceia sp., Fragilaria crotonensis, Fragilaria sp., Gleocapsa sp., Gloeothamnion sp., Hymenomonas sp., Isochrysis aff. galbana, Isochrysis galbana, Lepocinclis, Micractinium, Micractinium (UTEX LB 2614), Monoraphidium minutum, Monoraphidium sp., Nannochloris sp., Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrina, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Pascheria acidophila, Pavlova sp., Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pyramimonas sp., Pyrobotrys, Sarcinoid chrysophyte, Scenedesmus armatus, Schizochytrium, Spirogyra, Spirulina platensis, Stichococcus sp., Synechococcus sp., Tetraedron, Tetraselmis sp., Tetraselmis suecica, Thalassiosira weissflogii, and Viridiella fridericiana. In some embodiments, the algae is a Chlamydomonas species. In some embodiments, the algae is a Chlamydomonas reinhardtii. In some embodiments, the algae is a derivative of a green Chlamydomonas strain made by mutagenesis or by mating with another algae strain. In some embodiments, the Chlamydomonas sp. is strain CC-125 deposited to the University of Minnesota Chlamydomonas collection center or a derivative thereof.

### Culture Methods for Overproducing PPIX Strains

Methods for growing algae in liquid media include a wide variety of options including ponds, aqueducts, small scale laboratory systems, and closed and partially closed bioreactor systems. Algae can also be grown directly in water, for example, in an ocean, sea, lake, river, reservoir, etc.

In some embodiments, the PPIX overproducing algae useful in the methods and compositions provided herein are grown in a controlled culture system, such as a small scale laboratory system, a large scale system, and/or a closed and partially closed bioreactor system. Small scale laboratory systems refer to cultures in volumes of less than about 6 liters, and can range from about 1 milliliter or less up to about 6 liters. Large scale cultures refer to growth of cultures in volumes of greater than about 6 liters, and can range from about 6 liters to about 200 liters, and even larger scale systems covering 5 to 2500 square meters in area, or greater. Large scale culture systems can include liquid culture systems from about 10,000 to about 20,000 liters and up to about 1,000,000 liters.

The culture systems for use with the methods for producing the compositions herein include closed structures such as bioreactors, where the environment is under stricter control than in open systems or semi-closed systems. A photobioreactor is a bioreactor which incorporates some type of light source to provide photonic energy input into the reactor. The term bioreactor can refer to a system closed to the environment and having no direct exchange of gases and contaminants with the environment. A bioreactor can be described as an enclosed, and in the case of a photobioreactor, illuminated, culture vessel designed for controlled biomass production of liquid cell suspension cultures.

In some embodiments, the algae used in the methods and for the compositions provided herein are grown in fermentation vessels. In some embodiments, the vessel is a stainless steel fermentation vessel. In some embodiments, the algae are grown in heterotrophic conditions whereby one or more carbon sources is provided to the culture. In some embodiments, the algae are grown in aerobic and heterotrophic conditions. In some embodiments, the algae are grown to a density greater than or about 10 g/L, about 20 g/L, about 30 g/L, about 40 g/L, about 50 g/L, about 75 g/L, about 100 g/L, about 125 g/L or about 150 g/L.

In some embodiments, the algae are inoculated from a seed tank to a starting density of greater than about 0.1 g/L, about 1.0 g/L, about 5.0 g/L, about 10.0 g/L, about 20.0 g/L, about 50 g/L, about 80 g/L, or about 100 g/L. Once inoculated, the algae are grown heterotrophically using an aerobic fermentation process. During this process, the algae are fed nutrients to maintain their growth. In some embodiments, these nutrients include a reduced carbon source. Exemplary aerobic fermentation process and/or reduced carbon sources include, but are not limited to, acetate, glucose, sucrose, fructose, glycerol and other types of sugars (e.g., dextrose, maltose, galactose, sucrose, ribose, etc.). In some embodiments, the algae culture is supplemented with iron.

In some embodiments, the algae are cultured under dark conditions. Preferably, the dark condition has a brightness of less than 1000 lux, less than 750 lux, less than 500 lux, less than 400 lux, less than 300 lux, less than 200 lux, less than 100 lux. In some embodiments, the algae cultured under dark conditions lack or are reduced in chlorophyll production at least 10%, at least 20%, at least 30%, at least 40%, at least 50%, at least 60%, at least 70%, at least 80% compared to the algae cultured under dark conditions. In some embodiments, the algae grown under dark conditions are supplemented with one or more nutrients. In some embodiments, the algae grown under dark conditions are grown in the presence of a reduced carbon source, such as acetate, glucose, sucrose, fructose, glycerol or other types of sugars (e.g., dextrose, maltose, galactose, sucrose, ribose, etc.). In some embodiments, the algae grown under dark conditions are grown in the presence of iron or otherwise supplemented with iron.

### PPIX-Containing Preparations and Products

Algae strains and cultures overproducing PPIX such as described herein can be used in various forms and preparations. In some embodiments, a PPIX-containing composition is prepared from an algae culture overproducing PPIX, where the composition is red or red-like in color.

In some embodiments, the PPIX-containing composition is prepared from a biomass isolated from cultured algae. In some embodiments, the biomass is further fractionated to remove one or more components. In some embodiments, the biomass is fractionated to remove starch. In some embodiments, the biomass is fractionated to remove protein. In some embodiments, the biomass is fractionated or otherwise treated to remove carotenoids. In some embodiments, the biomass is fractionated or otherwise treated to enrich for certain components. In some embodiments, the fractionated or treated biomass is enriched in PPIX. In some embodiments, the fractionated or treated biomass is enriched in protein or in protein and PPIX. In some embodiments, the fractionation or treatment enhances the red or red-like color of the preparation. The fractionated or treated biomass can be enriched for protein content such that the composition is about 10% protein, greater than about 10% protein, or greater than about 20%, about 30%, about 40% or about 50% protein.

In some embodiments, the biomass is fractionated or otherwise treated to remove or reduce any heme content and optionally, to enrich for PPIX. Thus, in some embodiments, the fractionation or composition may include greater amount of PPIX than of heme by at least 10%, at least 20%, at least 30%, at least 40%, at least 50%. Such fractionation can include separation of PPIX from heme. For example, heme-binding proteins and heme associated with proteins can be separated from PPIX which is not a protein-conjugated or protein-associated compound. Both free heme and protein-associated heme can be separated from PPIX based on heme's association with iron. PPIX does not contain an iron moiety and as such, this feature can be used to separate PPIX from a heme-containing fraction. In some embodiments, an algae biomass herein is fractionated or otherwise treated such that the heme content is reduced, such as reduced below 1%, below 0.1%, below 0.05%, below 0.01%, below 0.001% or below a detectable level in a PPIX-containing fraction. Alternatively, an algae biomass herein is fractionated or otherwise treated such that heme-protein content is reduced, such as reduced below 1%, below 0.1%, below 0.05%, below 0.01%, below 0.001% or below a detectable level in a PPIX-containing fraction. In some embodiments, an algae biomass or fractionated biomass is produced from a strain deficient in ferrocheletase or in a strain that does not make or does not accumulate heme such that the biomass or fraction has little to no heme.

In some embodiments, the PPIX-containing composition is a PPIX-containing liquid prepared from the culture media of the cultured algae. In some embodiments, the PPIX-containing composition is prepared from PPIX found extracellularly in the algae culture (extracellular fraction). In some embodiments, the algae culture is lysed or otherwise treated to release PPIX from the cells. In some embodiments, the PPIX-containing liquid is further fractionated to remove one or more components. In some embodiments, the PPIX-containing liquid is fractionated to remove starch. In some embodiments, the PPIX-containing liquid is fractionated to remove protein. In some embodiments, the PPIX-containing liquid is fractionated or otherwise treated to remove carotenoids. In some embodiments, the PPIX-containing liquid is fractionated or otherwise treated to enrich for certain components. In some embodiments, the fractionated or treated PPIX-containing liquid is enriched in PPIX. In some embodiments, the fractionation or treatment enhances the red or red-like color of the preparation.

In some embodiments, the PPIX-containing liquid is fractionated or otherwise treated to remove or reduce any heme content and optionally, to enrich for PPIX. Such fractionation can include separation of PPIX from heme. For example, heme-binding proteins and heme associated with proteins in the liquid can be separated from PPIX which is not a protein-conjugated or protein-associated compound. Both free heme and protein-associated heme can be separated from PPIX based on heme's association with iron. PPIX does not contain an iron moiety and as such, this feature can be used to separate PPIX from a heme-containing fraction. In some embodiments, a PPIX-containing liquid is fractionated or otherwise treated such that the heme content is reduced, such as reduced below 1%, below 0.1%, below 0.05%, below 0.01%, below 0.001% or below a level that is generally detectable in a PPIX-containing fraction. In some embodiments, a PPIX-containing liquid is produced from a strain deficient in ferrocheletase or in a strain that does not make or does not accumulate heme such that the PPIX-containing liquid has little to no heme content.

In some embodiments, the biomass or PPIX-containing composition is a PPIX-containing liquid, and/or fractionated PPIX-containing composition or a PPIX-containing liquid contains protoporphyrin IX at about 0.5%, 1.0%, 1.5%, 2.0%, 2.5%, 3.0%, 3.5%, 4.5%, 5.0%, 5.5%, 6.0%, 6.5%, 7.0%, 7.5%, 8.0%, 8.5%, 9.0%, 9.5%, 10.0% or more than 10% on a weight per total weight basis. In addition, in some embodiments, the biomass or PPIX-containing composition is a PPIX-containing liquid, and/or fractionated PPIX-containing composition or a PPIX-containing liquid contains protoporphyrin IX greater than chlorophyll content by at least 5%, at least 10%, at least 20%, at least 30%, at least 40%, or at least 50%.

The PPIX-containing compositions, including biomass, liquid and fractionated preparations can be further processed. Such processing can include concentrating, drying, lyophilizing, and freezing. In various embodiments, the PPIX-containing compositions can be combined with additional components and ingredients, for example, to create an edible product. In some embodiments, the PPIX-containing composition confers a red or red-like color to the edible product. In some embodiments, the PPIX-containing composition confers a meat-like characteristic such as a meat-like taste, aroma and/or texture to the edible product. In some embodiments, the PPIX-containing composition provides the appearance of blood to an edible product, such as to a meat replica, a beef-like product, a chicken-like product or the like.

In some embodiments, PPIX-containing composition is a PPIX-containing liquid, and/or fractionated PPIX-containing composition or a PPIX-containing liquid provides at least 1%, 2%, 3%, 4%, 5%, 6%, 7%, 8%, 9% or 10% of the protein to the edible composition. In some embodiments, PPIX-containing composition is a PPIX-containing liquid, and/or fractionated PPIX-containing composition or a PPIX-containing liquid provides greater than 5%, 10%, 15%, 20%, 25%, 30%, 35%, 40%, 45%, 50%, 55%, 60%, 65%, 70%, 75%, 80%, 85%, 90%, 95%, 99% or 100% of the protein in the edible product. In some embodiments, PPIX-containing composition is a PPIX-containing liquid, and/or fractionated PPIX-containing composition or a PPIX-containing liquid provides a daily recommended dosage of omega-3 fatty acids or a portion thereof to the edible product, for example, at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 250 mg, 300 mg, 350 mg, 400 mg, 450 mg or 500 mg of omega-3 fatty acids to the edible composition.

In some embodiments, PPIX-containing composition is a PPIX-containing liquid, and/or fractionated PPIX-containing composition or a PPIX-containing liquid provides at least about 5%, 10%, 15%, 20%, 25%, 30%, 35%, 40%, 45%, 50%, 55%, 60%, 65%, 70%, 75%, 80%, 85%, 90%, 95%, 99% or 100% of the daily recommended dosage of vitamin A or at least about 20 μg, 50 μg, 100 μg, 200 μg, 300 μg, 400 μg, 500 μg, 600 μg, 700 μg, 800 μg, 900 μg or 1000 μg of retinol activity equivalents (RAE) for vitamin A. In some embodiments, PPIX-containing composition is a PPIX-containing liquid, and/or fractionated PPIX-containing composition or a PPIX-containing liquid provides no more than about 2,000 μg, 2,500 μg or 3,000 μg of retinol activity equivalents (RAE) for vitamin A. Alternatively and/or additionally, PPIX-containing composition is a PPIX-containing liquid, and/or fractionated PPIX-containing composition or a PPIX-containing liquid provides at least about 5%, 10%, 15%, 20%, 25%, 30%, 35%, 40%, 45%, 50%, 55%, 60%, 65%, 70%, 75%, 80%, 85%, 90%, 95%, 99% or 100% of the daily recommended dosage of beta-carotene. Alternatively and/or additionally, PPIX-containing composition is a PPIX-containing liquid, and/or fractionated PPIX-containing composition or a PPIX-containing liquid provides about 0.25 mg, 0.5 mg, 1 mg, 1.5 mg, 2 mg, 2.5 mg, 3 mg, 4 mg, 5, mg, 6 mg, 9 mg, 10 mg, 12 mg, or 15 mg of beta-carotene.

Alternatively and/or additionally, PPIX-containing composition is a PPIX-containing liquid, and/or fractionated PPIX-containing composition or a PPIX-containing liquid provides less than daily recommended limit for saturated fat or a portion thereof to the edible product, for example, no more than about 1%, 5%, 10%, 15%, 20%, 25%, 30%, 35%, 40%, 45%, 50%, 55%, 60%, 65%, 70%, 75%, 80%, 85%, 90%, 95%, or 100% of the daily recommended dosage of saturated fat. Alternatively and/or additionally, PPIX-containing composition is a PPIX-containing liquid, and/or fractionated PPIX-containing composition or a PPIX-containing liquid provides no more than 0.01%, 0.05%, 0.1%, 0.2%, 0.3%, 0.4%, 0.5%, 0.6%, 0.7%, 0.8%, 0.9%, 1.0%, 1.2%, 1.5%, 2%, 5% or 10% of total saturated fat present in the edible composition or in the finished product made from the edible composition.

In some embodiments, PPIX-containing compositions are combined with additional ingredients to create a meat-like product. Such meat-like products can include clean meat or cultured meat (made from animal cells grown in the laboratory or otherwise outside of an animal), plant-based and non-animal based meats (made from plant ingredients and/or ingredients not from animal sources). In some embodiments, a PPIX-containing composition made from an over-producing algae is combined with additional ingredients to create a meat-like product whereby the addition of the PPIX-containing composition confers a red or red-like color, a meat-like aroma, a meat-like taste and/or a meat-like texture to the meat-like product. In some embodiments, the meat-like features conferred by the PPIX-containing composition are conferred to the raw or uncooked product. In some embodiments, the meat-like features conferred by the PPIX-containing composition is conferred to the cooked product. Alternatively, least one of the features of meat or meat-like flavor or aroma, a meat or meat-like texture, a blood-like appearance, a meat or meat-like color are derived from the algae preparation.

In some embodiments, whole algae or fractionated algae is combined with an additional protein source in an edible composition. For example, the protein source may be wheat protein, such as wheat protein, textured wheat protein, pea protein, textured pea protein, soy protein, textured soy protein, potato protein, whey protein, yeast extract, a fungus protein such as quorn, or other plant-based protein source or any combination thereof. In some embodiments, whole algae or fractionated algae is combined with an oil or source of fat in an edible composition. For example, the oil or fat source may be coconut oil, canola oil, sunflower oil, safflower oil, corn oil, olive oil, avocado oil, nut oil or other plant-based oil or fat source or any combination thereof. In some embodiments, whole algae or fractionated algae is combined with a starch or other carbohydrate source such as from potato, chickpea, wheat, soy, beans, corn or other plant-based starch or carbohydrate or any combination thereof. In some embodiments, whole algae or fractionated algae is combined with a thickener in an edible composition. For example, starches such as arrowroot, cornstarch, katakuri starch, potato starch, sago, tapioca and their starch derivatives may be used as a thickener; microbial and vegetable gums used as food thickeners include alginin, guar gum, locust bean gum, konjac and xanthan gum; and proteins such as collagen and egg whites may be used as thickeners; and sugar polymers for use as thickeners include agar, methylcellulose, carboxymethyl cellulose, pectin and carrageenan. In some embodiments, whole algae or an algae fraction may be combined with vitamins and minerals in an edible composition, such as vitamin E, vitamin C, thiamine (vitamin B1), zinc, niacin, vitamin B6, riboflavin (vitamin B2), and vitamin B12.

In some embodiments, whole algae or an algae fraction may be combined with additional ingredients such that the edible composition and/or finished product is vegetarian, vegan or gluten-free, and therefore may conform to the dietary guidelines of Jewish kosher practitioners, and halal practitioners. Thus, in some embodiments, the edible composition and/or finished product may be suitable for consumption by vegetarians, vegans, gluten-free populations, Jewish kosher practitioners, and halal practitioners. In some embodiments, whole algae or an algae fraction may be combined with additional ingredients such that the edible composition and/or finished product is GMO-free and/or does not contain any ingredients derived from genetically engineered organisms or cells.

### EXEMPLARY NUMBERED EMBODIMENTS

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. A composition comprising a preparation from an algae strain, wherein the algae strain overexpresses or accumulates protoporphyrin IX (PPIX). 2. The composition of embodiment 2, wherein the preparation is a biomass from the algae strain. 3. The composition of embodiment 2, wherein the preparation is a fractionated biomass from the algae strain. 4. The composition of embodiment 3, wherein the fractionated biomass comprises a PPIX-enriched fraction. 5. The composition of embodiment 4, wherein the PPIX-enriched fraction further comprises a protein-enriched fraction. 6. The composition of embodiment 1, wherein the preparation is an extracellular fraction of the algae culture. 7. The composition according to any of embodiments 1-6, wherein the preparation is red or red-like in color. 8. The composition according to any of embodiments 1-7, wherein the preparation contains a greater amount of PPIX than of heme. 9. The composition according to any of embodiments 1-8, wherein the preparation contains less than about 1%, 0.5%, 0.1%, 0.05%, 0.01%, 0.005% or 0.001% heme. 10. The composition according to any of embodiments 1-9, wherein the preparation contains less than about 1%, 0.5%, 0.1%, 0.05%, 0.01%, 0.005% or 0.001% heme-protein. 11. The composition according to any of embodiments 1-10, wherein the preparation does not contain a detectable amount of a heme-protein. 12. The composition according to any of embodiments 1-11, wherein the preparation does not contain a detectable amount of heme. 13. The composition according to any of embodiments 1-4, wherein the preparation does not contain a detectable amount of protein. 14. The composition according to any of embodiments 1-13, wherein the preparation has a protoporphyrin IX content greater than chlorophyll content. 15. The composition according to any of embodiments 1˜4 and 6, wherein the preparation provides at least 5%, 10%, 15%, 20%, 25%, 30%, 35%, 40%, 45%, 50%, 55%, 60%, 65%, 70%, 75%, 80%, 85%, 90%, 95%, 99% or 100% of the total protein content to the edible composition. 16. The composition according to any of embodiments 1-15, wherein the preparation provides vitamin A, beta carotene or a combination thereof to the composition. 17. The composition of embodiment 16, wherein the vitamin A, the beta carotene or the combination thereof is at least about 5%, 10%, 15%, 20%, 25%, 30%, 35%, 40%, 45%, 50%, 55%, 60%, 65%, 70%, 75%, 80%, 85%, 90%, 95%, 99% or 100% of the daily recommended requirement. 18. The composition according to any of embodiments 1-17, wherein the preparation provides less than about 0.01%, 0.05%, 0.1%, 0.2%, 0.3%, 0.4%, 0.5%, 0.6%, 0.7%, 0.8%, 0.9%, 1.0%, 1.2%, 1.5%, 2%, 5% or 10% of total saturated fat present in the composition. 19. The composition according to any of embodiments 1-18, wherein the preparation provides at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 250 mg, 300 mg, 350 mg, 400 mg, 450 mg or 500 mg of omega-3 fatty acids to the composition. 20. The composition according to any of embodiments 1-19, wherein the composition has a red or red-like color derived from the preparation. 21. The composition according to any of embodiments 1-20, wherein the composition has a meat or meat-like flavor derived from the preparation. 22. The composition according to any of embodiments 1-21, wherein the composition has a meat or meat-like texture derived from the preparation. 23. The composition according to any of embodiments 1-22, wherein the algae is a Chlamydomonas sp. 24. The composition of embodiment 23, wherein the algae is Chlamydomonas reinhardtii. 25. The composition of embodiment 23, wherein the Chlamydomonas sp. is strain CC-125 deposited to the University of Minnesota Chlamydomonas collection center or a derivative thereof 26. The composition of embodiment 23, wherein the algae has reduced or absence of ferrocheletase activity or expression of ferrocheletase.

Embodiment 27. A food product comprising the composition according to any of embodiments 1-26. 28. The food product of embodiment 27, wherein the food product comprises a clean meat, a cultured meat, a synthetic meat, a plant-based meat or a non-animal cell-based meat. 29. The food product of any of embodiments 27-28, wherein the food product is selected from the group consisting of a beef-like food product, a fish-like product, a chicken-like product, a pork-like product and a meat replica. 30. The food product of any of embodiments 27-29, wherein the food product is vegan, vegetarian or gluten-free.

Embodiment 31. An edible ingredient comprising the composition according to any of embodiments 1-25. 32. The edible ingredient of embodiment 31, wherein the ingredient is part of a finished product, wherein the finished product has a red or red-like color derived from the ingredient. 33. The edible ingredient of embodiment 31, wherein the ingredient is part of a finished product, wherein the finished product has a meat or meat-like flavor derived from the ingredient. 34. The edible ingredient of embodiment 31, wherein the ingredient is part of a finished product, wherein the finished product Hasan appearance of blood derived from the ingredient. 35. The edible ingredient according to any of embodiments 31-34, wherein the finished product is an ingredient for a burger, a sausage, a kebab, a filet, a ground meat-like product or a meatball. 36. The edible ingredient according to any of embodiments 31-34, wherein the edible composition is part of a finished product and wherein the finished product is an animal feed. 37. The edible ingredient according to any of embodiments 31-36, wherein the edible ingredient is combined with a protein source, a fat source, a carbohydrate, a starch, a thickener, a vitamin, a mineral, or any combination thereof 38. The edible ingredient of embodiment 35, wherein the protein source is textured wheat protein, textured soy protein, fungal protein or algal protein. 39. The edible ingredient according to any of embodiments 37 or 38, wherein the finished product is free of animal proteins. 40. The edible composition of any of embodiments 37-39, wherein the fat source comprises at least one of refined coconut oil or sunflower oil. 41. The edible composition of any of embodiments 37-40, further comprising at least one of potato starch, methylcellulose, water, and a flavor, wherein the flavor is selected at least one of yeast extract, garlic powder, onion powder, and salt.

Embodiment 42. A meat substitute comprising the composition according to any of embodiments 1-26 or the edible ingredient according to any of embodiments 31-41. 43. The meat substitute of embodiment 42, further comprising: (a) 0.01%-5% (by weight of the meat replica matrix) of a non-animal protoporphyrin IX; (b) a compound selected from glucose, ribose, fructose, lactose, xylose, arabinose, glucose-6-phosphate, maltose, and galactose, and any combination thereof; (c) at least 1.5 mM of a compound selected from cysteine, cystine, thiamine, methionine, and any combination thereof; and (d) one or more proteins selected from the group consisting of plant proteins, fungal proteins and algal proteins, wherein the meat substitute is a ground beef-like food product that contains no animal product; wherein cooking the ground beef-like food product results in the production of at least two volatile compounds which have a beef-associated aroma.

Embodiment 44. A method of producing a protoporphyrin IX composition, comprising: growing an algae population comprising an algae that is a protoporphyrin IX over-producer; and isolating a protoporphyrin IX composition from the culture. 45. The method of embodiment 44, wherein the growing comprises culturing the algae culture in an aerobic fermentation condition. 46. The method of embodiment 44 or embodiment 45, wherein the algae contains a chloroplast. 47. The method of embodiment 46, wherein biosynthesis of the protoporphyrin IX occurs in the chloroplast. 48. The method according to any of embodiments 44-47, wherein the algae is deficient for its ability to produce chlorophyll. 49. The method according to any of embodiments 44-48, wherein the algae is deficient for the ability to produce a functional Mg-chelatase enzyme. 50. The method according to any of embodiments 44-49, wherein the algae is reduced in or lacks ChlD1, ChlD2 or ChlDH. 51. The method according to any of embodiments 44-50, wherein the algae is reduced in or lacks a functional light dependent protochlorophyllide. 52. The method according to any of embodiments 44-51, wherein the algae is reduced in or lacks a functional light independent protochlorophyllide. 53. The method according to any of embodiments 44-52, wherein the algae is reduced in or lacks ChlB, ChlL, or ChlN. 54. The method according to any of embodiments 44-53, wherein the algae overexpresses one or more of glutamyl-tRNA reductase, glutamate-1-semialdehyde aminotransferase, ALA dehydratase, porphobilinogen deaminase, UPG III synthase, UPG III decarboxylase, CPG oxidase, and PPG oxidase. 55. The method according to any of embodiments 44-54, wherein the algae is generated by mating and wherein the generated strain is red or red-like in color. 56. The method according to any of embodiments 44-55, wherein the algae is generated by mutagenesis. 57. The method according to any of embodiments 44-56, wherein the algae is red or red-like in color. 58. The method according to any of embodiments 44-57, wherein the isolated protoporphyrin IX composition is an algae biomass. 59. The method of embodiment 58, wherein the algae biomass is fractionated. 60. The method of embodiment 59, wherein the algae biomass is fractionated to produce a protein-enriched fraction comprising protoporphyrin IX. 61. The method according to any of embodiments 44-57, wherein the isolated protoporphyrin IX composition is isolated from extracellular media of the algae culture. 62. The method according to any of embodiments 44-61, wherein the isolated protopophyrin IX composition is isolated away from algae protein. 63. The method according to any of embodiments 44-62, wherein the algae is deficient for carotenoids. 64. The method according to any of embodiments 44-63, wherein the algae is a Chlamydomonas sp. 65. The method according to any of embodiments 44-64, wherein the algae is a Chlamydomonas reinhardtii. 66. The method of embodiment 64, wherein the Chlamydomonas sp. is strain CC-125 deposited to the University of Minnesota Chlamydomonas collection center or a derivative thereof 67. The method according to any of embodiments 44-66, wherein progeny of a protoporphyrin IX overexpressing algae strain grows faster than its parental strain following a mating with another algae. 68. The method according to any of embodiments 44-66, wherein the protoporphyrin IX algae is a strain produced by mating a strain deficient for carotenoids with a strain exhibiting a red or red-like color. 69. The method according to any of embodiments 44-66, wherein the protoporphyrin IX overexpressing algae is generated by mutagenesis of a first starting strain and selection of a second strain that grows faster in the dark than the first starting strain. 70. The method according to any of embodiments 44-66, wherein the protoporphyrin IX algae is generated by mutagenesis of a first strain and selection of a second strain that lacks one or more carotenoids from the mutated first strain. 71. The method according to any of embodiments 44-70, wherein the algae lacks a functional ferrocheletase enzyme. 72. The method according to any of embodiments 44-70, wherein the algae is reduced in the amount of, or activity of, a ferrocheletase enzyme. 73. The method according to any of embodiments 44-70, wherein the algae is reduced in the amount of or lacks heme as compared to a wildtype strain.

Embodiment 74. A protoporphyrin IX-containing composition produced by the method according to any of embodiments 44-73. 75. The composition of embodiment 74, wherein the composition does not contain a detectable level of heme, heme-binding protein or a combination thereof 76. The method according to any of embodiments 44-73, further comprising the steps of: (a) culturing the algae strain under a dark condition, wherein the strain does not produce or is reduced in the production of chlorophyll, and (b) collecting a portion of the algae culture that is red or red-like in color to produce the protoporphyrin IX composition. 77. The method of embodiment 76, wherein the algae is a Chlamydomonas sp. 78. The method of embodiment 77, wherein the algae is a Chlamydomonas reinhardtii. 79. The method according to embodiment 76, wherein the algae exhibits a red or red-like color when grown in the dark condition. 80. The method of embodiment 76, wherein the collected portion is extracellular media from the algae culture. 81. The method of embodiment 76, wherein the collected portion is a biomass or fractionated biomass from the algae culture. 82. The method of embodiment 76, wherein the algae is grown in an aerobic fermentation condition. 83. The method of embodiment 76, wherein the algae is grown to a density greater than 10 g/L, 20 g/L, 30 g/L, 40 g/L, 50 g, L, 75 g/L, 100 g/L, 125 g/L, or 150 g/L. 84. The method of embodiment 76, wherein the algae is grown with acetate as a reduced carbon source. 85. The method of embodiment 76, wherein the algae is grown with sugar as a reduced carbon source. 86. The method of embodiment 76, wherein the algae culture is supplemented with iron during the culturing step. 87. The method of embodiment 76, wherein the algae culture is inoculated at a density greater than 0.1 g/L, 1.0 g/L, 5.0 g/L, 10 g/L, 20 g/L, 50 g/L, 80 g/L, or 100 g/L. 88. The method of embodiment 76, further comprising fractionating the collected portion, wherein the fractionating removes from the collected portion substantially all or most of a component selected from the group consisting of carotenoids, starch, and protein. 89. The method of embodiment 76, further comprising fractionating the collected portion, wherein the fractionating removes from the collected portion substantially all or most of heme, heme-binding protein or a combination thereof 90. The method of embodiment 76, further comprising fractionating the collected portion, wherein the fractionating produces a protein-enriched fraction. 91. The method of embodiment 76, wherein the algae lacks or is reduced in one or more of magnesium chelatase, magnesium protoporphyrinogen IX, protochlorophyllide, chlorophyllide, and chlorophyll. 92. The method of embodiment 76, wherein the algae lacks or is reduced in ferrocheletase. 93. The method according to any of embodiments 44-73 and 76-92, wherein the algae is not a transgenic strain.

Embodiment 94. A clean meat product produced by the method according to any of embodiments 76-93, wherein the process further comprises combining the collected portion with a clean meat manufacturing composition, wherein the collected portion provides a red or red-like color to the clean meat product. 95. The clean meat product of embodiment 94, wherein the collected portion is a PPIX-enriched fraction or purified PPIX.

Embodiment 96. An edible ingredient produced by the method according to any of embodiments 44-73 and 76-93, wherein the protoporphyrin IX composition confers a meat or meat-like flavor, texture, odor or any combination thereof to the edible ingredient. 97. The edible ingredient of embodiment 96, wherein the edible ingredient is incorporated in a finished product selected from the group consisting of a beef-like food product, a fish-like product, a chicken-like product, a pork-like product and a meat replica. 98. The edible ingredient of any of embodiments 96-97, wherein the edible ingredient is vegan, vegetarian or gluten-free. 99. The edible ingredient of any of embodiments 96-98, wherein the edible ingredient is free of animal proteins. 100. The edible ingredient of any of embodiments 96-99, wherein the edible ingredient does not contain any genetically modified components.

Embodiment 101. An engineered algae having a genetic modifications, where the genetic modification results in an accumulation of protoporphyrin IX (PPIX) in the algae as compared to an algae lacking the genetic modification. 102. The engineered algae of embodiment 101, wherein the engineered algae has reduced or absence of chlorophyll production. 103. The engineered algae of embodiments 101 or 102, wherein the algae has red or red-like color. 104. The engineered algae according to any of embodiments 101-103, wherein the algae is capable of growth on glucose as the sole carbon source. 105. The engineered algae according to any of embodiments 101-104, wherein the genetic modification comprises a genetic alteration to chlorophyll synthesis pathway, protoporphyrinogen IX synthesis pathway or heme synthesis pathway. 106. The engineered algae according to any of embodiments 101-105, wherein the genetic modification is associated with a deficiency in the expression of ferrocheletase. 107. The engineered algae according to any of embodiments 101-106, wherein the genetic modification comprises an alteration in one or more of CHLD, CHLI1, CHLI2 or CHLH1. 108. The engineered algae of embodiments 106 or 107, wherein the genetic modification comprises an alteration in an upstream regulatory region, a downstream regulatory region, an exon, an intron or any combination thereof 109. The engineered algae according to any of embodiments 105-108, wherein the genetic modification comprises an insertion, a deletion, a point mutation, an inversion, a duplication, a frameshift or any combination thereof.

Embodiment 110. The engineered algae according to any of embodiments 101-109, wherein the engineered algae has a PPIX content greater than the chlorophyll content. 111. The engineered algae according to any of embodiments 101-109, wherein the engineered algae has a heme content greater than the chlorophyll content. 112. The engineered algae according to any of embodiments 101-111, wherein the engineered algae has reduced production of one or more fatty acids. 113. The engineered algae according to any of embodiments 101-112, wherein the engineered algae further comprises a genetic modification that reduces or eliminates the expression of light independent protochlorophyllide oxidoreductase. 114. The engineered algae of embodiment 113, wherein the genetic modification comprises a mutation or deletion in one or more of ChlB, ChlL or ChlN. 115. The engineered algae according to any of embodiments 101-114, wherein the engineered algae has down-regulated expression of ferrocheletase. 116. The engineered algae according to any of embodiments 101-115, wherein the engineered algae has upregulated expression of protoporphyrinogen IX oxidase.

Embodiment 117. The engineered algae according to any of embodiments 101-116, wherein the algae contain a recombinant or heterologous nucleic acid. 118. The engineered algae according to any of embodiments 101-117, wherein the engineered algae comprises a Chlamydomonas sp. 119. The engineered algae of embodiment 118, wherein the Chlamydomonas sp. is Chlamydomonas reinhardtii.

Embodiment 120. An edible composition comprising an algae preparation, wherein the algae preparation comprises an engineered algae of any of embodiments 101-119 or a portion thereof 121. The edible composition of embodiment 120, wherein the edible composition comprises PPIX derived from the engineered algae. 122. The edible composition of embodiment 120, wherein the algae preparation comprises algae cells. 123. The edible composition of embodiment 120, wherein the algae preparation is a fractionated algae preparation.

### EXAMPLES

### Example 1: Identification of PPIX-Overproducing Algae

Strains of algae (Chlamydomonas reinhardtii) overexpressing PPIX were identified by their inability to produce chlorophyll. Additionally, these strains exhibited red, brown, orange or some variation of the listed color. The identified strains exhibit light sensitivity and cannot be grown in direct light greater than 10 μE m⁻² s⁻¹ for extended periods of time.

One of the identified strains was grown under fed-batch aerobic fermentation conditions where acetate is used as a reduced carbon source of nutrition for the culture. The strain was grown in a fermenter where minimal light can reach the culture. The strain was grown to a density that is greater than 50 g/1_, and harvested via centrifugation. The harvested strain was red in color and can be added to compositions, such as food products, to confer a red, orange or brown color.

Tables 1-5 show characteristic analysis of one exemplary, identified red heme algae (Strain number: TAI114, Species name: Chlamydomonas reinhardtii).

TABLE 1
MICROBIAL ANALYSIS

| Quality Measure | Specification | Result | Units | Method | Conclusion |
|---|---|---|---|---|---|
| Aerobic Plate Count | ≤10,000 | 7,250 | CFU · g − 1 | AOAC 990.12 | Specification Met |
| E. coli (Generic) | Negative | Negative | CFU · g − 1 | AOAC 991.14 | Specification Met |
| Total coliforms | ≤1,000 | Negative | CFU · g − 1 | AOAC 991.14 | Specification Met |
| Salmonella | Negative | Negative | ORG · 25 g | AOAC 030301 | Specification Met |
| Staphylococcus | Negative | Negative | CFU · g − 1 | AOAC 2003.07 | Specification Met |
| aureus | | | | | |
| Pseudomonas | Negative | Negative | CFU · g − 1 | USP | Specification Met |
| aeruginosa | | | | | |

TABLE 2
HEAVY METAL ANALYSIS

| Quality Measure | Specification | Result | Units | Method | Conclusion |
|---|---|---|---|---|---|
| Arsenic | ≤0.01 ppm | ≤0.01 ppm | ppm | MET-CH-030 | Specification Met |
| Cadmium | ≤0.1 ppm | ≤0.01 ppm | ppm | MET-CH-030 | Specification Met |
| Lead | ≤0.01 ppm | ≤0.01 ppm | ppm | MET-CH-030 | Specification Met |
| Mercury | ≤0.005 ppm | ≤0.01 ppm | ppm | MET-CH-030 | Specification Met |
| Sulfite | ≤10 ppm | ≤0.01 ppm | ppm | MET-NHP-018 | Specification Met |

TABLE 3
BIOMASS ANALYSIS

| Quality Measure | Result | Unit |
|---|---|---|
| Moisture | 10.66 | Percent of biomass |
| Ash | 3.19 | Percent of biomass |
| Protein | 26.00 | Percent of biomass |
| Fat | 4.77 | Percent of biomass |
| Starch | 39.5 | Percent of biomass |
| Soluble Dietary Fiber | 8.85 | Percent of biomass |
| Insoluble Dietary Fiber | 1.15 | Percent of biomass |

TABLE 4
Porphyrin (Heme) ANALYSIS

| Quality Measure | Result | Unit |
|---|---|---|
| Heme | 0.60 | Percent |
| protoporphyrin IX | 4.60 | Percent |

TABLE 5
AMINO ACID COMPOSITION

| Amino Acid | Result | Unit |
|---|---|---|
| Alanine | 2.25 | Percent of biomass |
| Arginine | 2.03 | Percent of biomass |
| Asparagine/Aspartic Acid | 2.38 | Percent of biomass |
| Glycine | 1.49 | Percent of biomass |
| Cysteine | 0.48 | Percent of biomass |
| Glutamine/glutamic acid | 2.83 | Percent of biomass |
| Proline | 1.63 | Percent of biomass |
| Serine | 1.25 | Percent of biomass |
| Tyrosine | 1.05 | Percent of biomass |
| Histidine | 0.51 | Percent of biomass |
| Isoleucine | 1.04 | Percent of biomass |
| Leucine | 2.38 | Percent of biomass |
| Lysine | 1.78 | Percent of biomass |
| Methionine | 0.63 | Percent of biomass |
| Phenylalanine | 1.15 | Percent of biomass |
| Threonine | 0.83 | Percent of biomass |
| Tryptophan | 0.55 | Percent of biomass |
| Valine | 1.88 | Percent of biomass |
| Percent Non-Essential | 51.1 | Percent of protein |
| Amino Acids | | |
| Percent Amino Acids | 48.9 | Percent of protein |

### Example 2: Fractionation

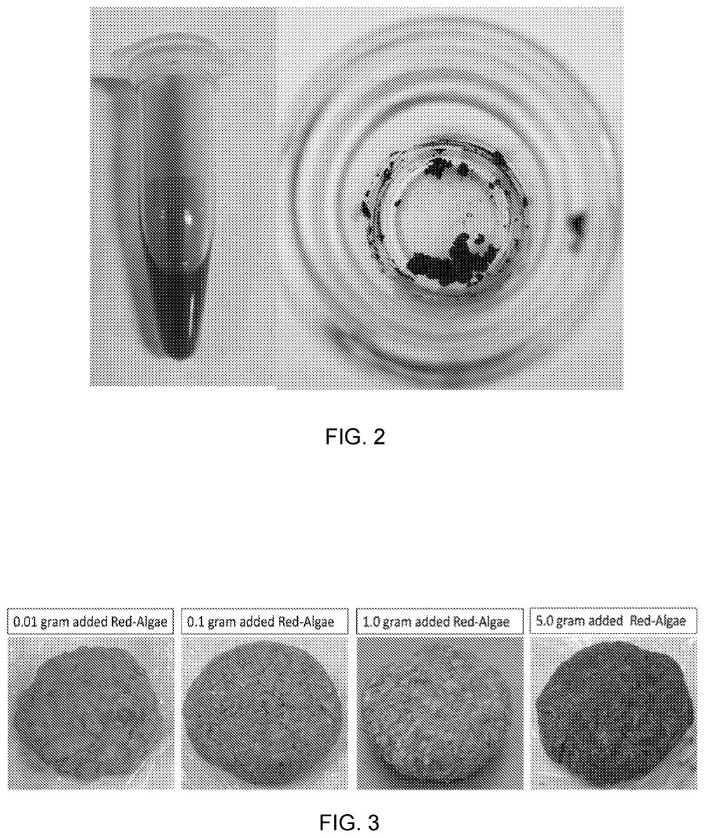

Cells from a PPIX overproducing strain of Chlamydomonas reinhardtii were harvested from a fermentation culture. Cells were re-suspended in a 10 mL solution (8:2 v/v solution of acetone:1.6M HCL) and vortexed for 30 minutes. The cell debris was centrifuged and the porphyrin layer was separated from the cell debris (FIG. 2). The porphyrin fraction was then diluted 1:10 with H₂O to precipitate the porphyrins from the acetone solution. Samples were further washed with water and can be frozen at that point for further analysis. Shown in FIG. 2 is the PPIX-containing fraction after the final step.

### Example 3: Preparation of a PPIX-Enriched “Meatless” Burger

The PPIX-enriched samples were used to prepare compositions of meat-like products produced from plant based materials and algae rich in PPIX. To create a PPIX-enriched burger, ingredients were mixed in the following proportions and formed into a disc shaped algae-plant based burger: 20% or about 20% Textured wheat protein, 20% or about 20% Refined coconut oil, 3% or about 3% Sunflower oil, 2% or about 2% Potato starch, 0.5% or about 0.5% Kojac gum, 0.5% or about 0.5% Xanthan gum, 45% or about 20% water and 4-9% or about 4-9% Flavors, including yeast extract, garlic powder, onion powder, salt, and PPIX-enriched (“red”) algae. Shown in FIG. 3 are burgers created with 0.01 g, 0.1 g, 1.0 g, and 5.0 g of the PPIX enriched algae.

In this example, the composition of the PPIX-enriched algae was 4.5% PPIX, 0.5% heme, 0% chlorophyll, 24.4% protein, 9% dietary fiber, 40% starch, 0.8% omega-3-fatty acids, 3.9% other fats, 7.5% moisture, and 8.4% ash.

### Example 4: Preparation of a PPIX-Enriched Plant-Based Burger

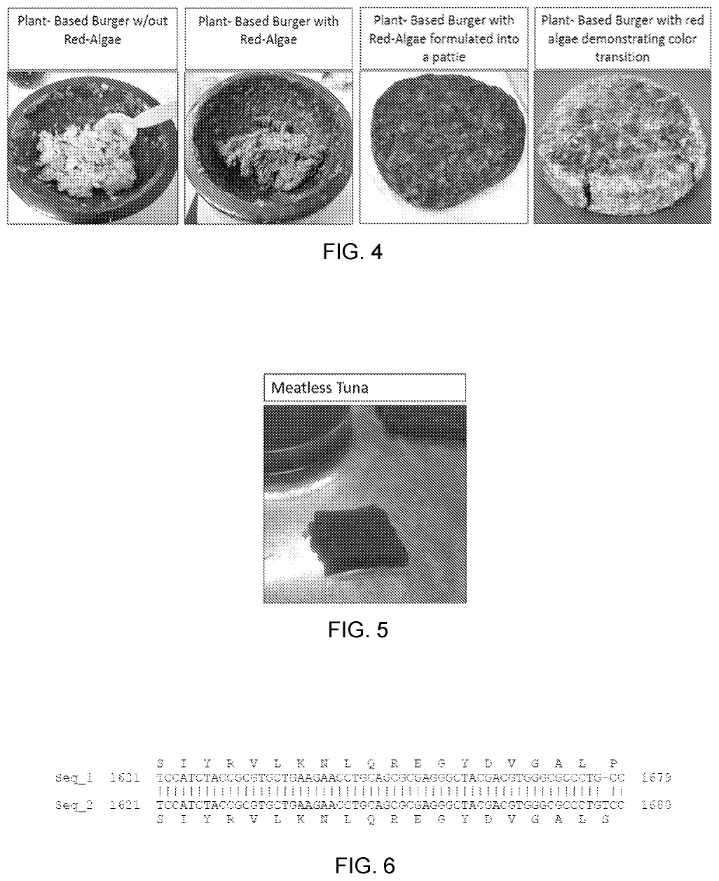

The PPIX-enriched samples was used to prepare burger compositions from plant based materials and algae rich in PPIX. To create a PPIX-enriched plant-based burger, ingredients were mixed in the following proportions and formed into a disc: 20% or about 20% Textured soy protein, 20% or about 20% Refined coconut oil, 3% or about 3% Sunflower oil, 2% or about 2% Potato starch, 1% or about 1% methylcellulose, 45% or about 45% water and 4-9% or about 4-9% Flavors, including yeast extract, garlic powder, onion powder, salt, and PPIX-enriched (“red”) algae. Shown in FIG. 4 are the ingredient mixes of the plant-based burger ingredients with no PPIX-enriched algae (far left), with the addition of PPIX-enriched algae (second from left), the ingredients with the addition of PPIX-enriched algae shaped into a burger before and after cooking (third from left and far right photos, respectively). As shown, the addition of the PPIX-enriched algae conferred a red/red-like color (resembling a burger with animal blood) to the ingredient mix and to the burger, and this color undergoes a transition when cooked.

In this example, the composition of the PPIX-enriched algae was 4.5% PPIX, 0.5% heme, 0% chlorophyll, 24.4% protein, 9% dietary fiber, 40% starch, 0.8% omega-3-fatty acids, 3.9% other fats, 7.5% moisture, and 8.4% ash.

### Example 5: Preparation of a PPIX-Enriched Meatless “Tuna”

The PPIX-enriched samples were used to prepare fish-like compositions. To create a PPIX-enriched meatless “fish”, ingredients were mixed in the following proportions: 20% Textured soy protein, 65% water and 10% Flavors and PPIX-enriched 5% (“red”) algae. Shown in FIG. 5 is a square portion of the meatless “tuna” produced.

In this example, the composition of the PPIX-enriched algae was 4.5% PPIX, 0.5% heme, 0% chlorophyll, 24.4% protein, 9% dietary fiber, 40% starch, 0.8% omega-3-fatty acids, 3.9% other fats, 7.5% moisture, and 8.4% ash.

### Example 6: Separation of PPIX from Heme and Heme-Binding Proteins

Separation of PPIX from heme and heme-binding proteins may be accomplished as follows. Algae biomass is mixed in a buffer such as Tris-EDTA buffer (pH 7.2) and stirred for 1 h at room temperature at about 1600 RPM. Samples are then placed on ice and treated with ultrasonication such as for 5 min with 1 s pulse. To the ultrasonicated algae, acetonitrile is added and then the mixture is vortexed for about 5 min and then is subjected to centrifugation such as at 2500×g for 5 min, making a pellet of precipitated proteins. The acetonitrile containing supernatant containing porphyrins (including PPIX), is removed and can be analyzed for porphyrin content and further use.

Further separation can be achieved as follows: To the pellet from the centrifugation, acetonitrile:1.7 M HCl (8:2, v/v) is added and placed in a shaker for about 20 min, extracting heme from the proteins into the acetonitrile. To create a two phase liquid-liquid system, saturated MgSO₄ and NaCl are added, and the solution is then vortexed for about 5 min and centrifuged at 2500×g for 5 min. The top organic layer can be removed and, if necessary, diluted with pure acetonitrile prior to analysis and further separation by LC/MS-MS. (Fyrestam and Ostman, Anal Bioanal Chem (2017) 409:6999-7010 “Determination of heme in microorganisms using HPLC-MS/MS and cobalt(III) protoporphyrin IX inhibition of heme acquisition in Escherichia coli”).

### Example 7: Targeted Modification of Chlorophyll Pathway to Create PPIX-Enriched Strains

Guide RNAs (sgRNAs) can be designed against the ferrocheletase gene to cause a deletion or an insertion that renders the protein complex non-functional, including a modification to one or more of SEQ ID NOs: 116-122. Once designed sgRNAs can be combined with the Cas9 protein by incubating them at 37° C. to form ribonuclear proteins (RNPs). These RNPs carrying the sgRNAs to target ferrocheletase are then electroporated into green algae cultures. 3×10⁸ cells are placed into MAX efficiency transformation buffer reagent for algae (Thermo fisher scientific) and placed into a cuvette with a 0.2 cm gap. The electroporation voltage is set to 250V and the pulse interval is set to 15 ms. Once electroporated cells are recovered in growth media with 40 mM sucrose added to improve recovery efficiency. Cells are then plated on growth media containing agar and grown in the dark due to the photosensitivity of chlorophyll-deficient mutants. Once recovered the population can be pulled and struck out for individual colonies. Plates are again placed in the dark for 2 to 3 weeks. Mutants of ferrocheletase can be identified by an increase in their fluorescence at 635 nm when they are excited by a light with a wavelength of 420 nm when compared to the unmodified green algae.

### Example 8: Additional Modification of PPIX-Enriched Strains that are Improved for Different Meat Imitations and Other Uses

Strains of algae overexpressing PPIX can by mated with strains that are under or overproducing omega-3s, omega-6s or omega-9s. For imitation fish, more omega oils in strains of algae overexpressing heme are ideal. For imitation beef-like products, less omega oils in strains of algae overexpressing heme are ideal. As such strains of algae that are mutants for either over or underexpressing omega oils can be mated with strains of algae overexpressing heme to form a more ideal algae for various meat-like products.

Mating can be done by identifying strains of Chlamydomonas that are the opposite mating type and then starving them for nitrogen. After nitrogen starvation, strains are re-suspended in water to promote the formation of flagella. The flagella of the different mating types assist in the fusion of algae strains that will result in the formation of a zygote. The mated cultures are then exposed to chloroform to kill strains that did not mate. The chloroform does not kill zygotes. The zygotes are then placed into growth medium and allowed to propagate. Individual colonies are then identified and screened for an increase in PPIX by measuring for an increase in fluorescence of the precursor protoporphyrin IX or by biochemical assay (Abnova KA1617) as well as those that are overexpressing or under-expressing omega oils.

SEQUENCES

ALA dehydratase (ALAD) nucleic acid sequence (SEQ ID NO: 1):
atgcagatgatgcagcgcaacgttgtgggccagcgccccgtcgctggctcccgccgctcgctggtggttgccaac
gttgcggaggtgacccgccccgcggtcagcaccaacggcaagcaccggactggtgtgccggagggaactcccatc
gtcacccctcaggacctgccctcgcgccctcgccgcaaccgccgcagcgagagcttccgtgcttccgttcgtgag
gtgaacgtgtcgcccgccaacttcatcctgccgatcttcatccacgaggagagcaaccagaacgtgcccatcgcc
tccatgcctggcatcaaccgcctggcgtatggcaagaacgtgattgactacgttgctgaggctcgctcttacggt
gtcaaccaggtcgtggttttccccaagacgcccgaccacctgaagacgcaaaccgcggaggaggcgttcaacaag
aacggcctcagccagcgcacgatccgcctgctgaaggactctttccctgacctggaggtgtacacggacgtggct
ctggacccctacaactcggacggccacgacggtatcgtgtcggacgccggtgtgatcctgaacgacgagaccatc
gagtacctgtgccgccaggccgtgagccaggccgaggccggtgccgacgtggtgtcgccctctgacatgatggac
ggccgcgtgggcgccatccgccgcgccctggaccgcgagggcttcaccaacgtgtccatcatgtcctacaccgcc
aagtacgcctccgcctactacggccccttccgtgacgccctggcgtccgcgcccaagcccggccaggcgcaccgc
cgcatcccccccaacaagaagacctaccagatggaccccgccaactaccgcgaggccatccgcgaggccaaggcc
gacgaggccgagggcgctgacatcatgatggtcaagcccggcatgccgtacctggacgtggtacgcctgctgcgt
gagaccagcccgctgcccgtggccgtgtaccacgtgtcgggcgagtacgccatgctcaaggcggcggcggagcgc
ggctggctgaacgagaaggatgccgtgcttgaggccatgacctgcttccgccgcgccggcgctgacctcatcctc
acctactacggcattgaggcctccaagtggctggcgggcgagaagtaa
ALA dehydratase (ALAD) amino acid sequence (SEQ ID NO: 2):
MQMMQRNVVGQRPVAGSRRSLVVANVAEVTRPAVSTNGKHRTGVPEGTPIVTPQDLPSRPRRNRRSESFRASVRE
VNVSPANEILPIFIHEESNQNVPIASMPGINRLAYGKNVIDYVAEARSYGVNQVVVFPKTPDHLKTQTAEEAFNK
NGLSQRTIRLLKDSFPDLEVYTDVALDPYNSDGHDGIVSDAGVILNDETIEYLCRQAVSQAEAGADVVSPSDMMD
GRVGAIRRALDREGFTNVSIMSYTAKYASAYYGPFRDALASAPKPGQAHRRIPPNKKTYQMDPANYREAIREAKA
DEAEGADIMMVKPGMPYLDVVRLLRETSPLPVAVYHVSGEYAMLKAAAERGWLNEKDAVLEAMTCFRRAGADLIL
TYYGIEASKWLAGEK
coproporphyrinogen III oxidase (CPX1) nucleic acid sequence (SEQ ID NO: 3):
atggcactgcaagcctcaacccgctcgctccagcagcgccgcgccttctcttcggcccagacctccaagcgtgtg
tctgtgaccaaggtccgcgcgacggctatcgaggcggagaactatgtgaagcaggctccccagtcgctggtccgc
ccgggcatcgacactgaggactctatgcgcgctcgcttcgagaaggtgatccgcaacgcccaggactccatctgc
aatgctatctccgagatcgatggcaagccgttccaccaggacgcctggacccgccccggcggcggtggcggcatc
agccgcgtgctgcaggacggcaacgtgtgggagaaggccggcgtcaacgtgtccgtggtctacggcaccatgccc
cctgaggcctaccgcgctgccactggcaacgccgagaagctgaagaacaagggtgacggtggccgcgtgcccttc
ttcgccgccggcatctcgtcggtgatgcacccccgcaacccccactgccccaccatgcacttcaactaccgctac
ttcgagactgaggagtggaacggcatccccggccagtggtggttcggcggcggcaccgacatcacccccagctat
gtggtgcccgaggacatgaagcacttccacggcacctacaaggcggtgtgcgaccgccacgatcccgcttactac
gagaagttccgcacctggtgcgatgagtacttcctcatcaagcaccgcggcgagcgccgcggcctgggcggcatc
ttcttcgatgacctgaacgaccgcaaccccgaggacatcctgaagttctcgaccgacgccgtgaacaacgtggtg
gaggcatactgccccatcatcaagaagcacatgaacgacccctacacccccgaggagaaggagtggcagcagatc
cgccgcggccgctacgtggagttcaacctggtctatgaccgcggcaccaccttcggcctgaagaccggcggccgc
attgagtcgatcctcatgtccatgccccagaccgcctcatggctgtacgaccaccagcccaaggccggctcgccc
gaggccgagctgctcgacgcctgccgcaacccccgcgtctgggtgtaa
coproporphyrinogen III oxidase (CPX1) amino acid sequence (SEQ ID NO: 4):
MALQASTRSLQQRRAFSSAQTSKRVSVTKVRATAIEAENYVKQAPQSLVRPGIDTEDSMRARFEKVIRNAQDSIC
NAISEIDGKPFHQDAWTRPGGGGGISRVLQDGNVWEKAGVNVSVVYGTMPPEAYRAATGNAEKLKNKGDGGRVPF
FAAGISSVMHPRNPHCPTMHFNYRYFETEEWNGIPGQWWFGGGTDITPSYVVPEDMKHFHGTYKAVCDRHDPAYY
EKERTWCDEYFLIKHRGERRGLGGIFFDDLNDRNPEDILKFSTDAVNNVVEAYCPIIKKHMNDPYTPEEKEWQQI
RRGRYVEFNLVYDRGTTFGLKTGGRIESILMSMPQTASWLYDHQPKAGSPEAELLDACRNPRVWV
coproporphyrinogen III oxidase (CPX2) nucleic acid sequence (SEQ ID NO: 5):
atgctgaggaagcagattggtggatctggccagcagcgggcgggcctccgacgggtgaaccaaggacctgcgcgt
cggcggttggcaccctgccgcgtggcggcccccgtgcaaacctcgtcctccgtcgccacattcaatggcttcgtg
gactacattcacggactccagaagaacattctgagcactgctgaggatctggagaacggcgagcggaagtttgtt
gttgaccgctgggagcgcgacgccagcaaccccaacgccgggtatggcattacgtgcgtgcttgaggacgggaag
gtgctggagaaggccgcagccaatatctcagtggtgcgcgggacgctgtcggcgcagcgcgcagtggccatgagc
tcccgcggccgcagcagcatcgaccccaagggcgggcagccctacgccgcggccgccatgagcctagtgttccac
agcgcgcacccgctcatccccacgctgcgcgcgacgtgcggttgttccaggtgggcgatgaggcgtggtacggcg
gtggctgtgacctgacgcccaactacctagacgtggaggactcgcagtccttccaccgctactggaaggacgtgt
gcggcaagtacaagccgggcctgtacaccgagctcaaggagtggtgcgacaggtacttctacatcccggcccgca
aagagcaccgtggcattggcggcctgttctttgatgacatggccactgcggaggcgggctgcgatgtggaggcgt
ttgtgcgggaagtgggagatggcatcctgccctgctggctgcccatcgtggcgcggcaccgtggccagcccttca
cggagcagcagcggcaatggcagctgctgcgccgcggtcgctacatcgagttcaacctgctgtacgaccgcggca
tcaagttcggtctggacggcggccgcatcgagagcatcatggtgtcggcgccgccgctgatcgcgtggaagtaca
acgtggtgccacagccgggcagccccgaggaggagatgctgaaggtgcttcagcagccccgcgagtgggcctga
coproporphyrinogen III oxidase (CPX2) amino acid sequence (SEQ ID NO: 6):
MLRKQIGGSGQQRAGLRRVNQGPARRRLAPCRVAAPVQTSSSVATFNGFVDYIHGLQKNILSTAEDLENGERKFV
VDRWERDASNPNAGYGITCVLEDGKVLEKAAANISVVRGTLSAQRAVAMSSRGRSSIDPKGGQPYAAAAMSLVFH
SAHPLIPTLRADVRLFQVGDEAWYGGGCDLTPNYLDVEDSQSFHRYWKDVCGKYKPGLYTELKEWCDRYFYIPAR
KEHRGIGGLFFDDMATAEAGCDVEAFVREVGDGILPCWLPIVARHRGQPFTEQQRQWQLLRRGRYIEFNLLYDRG
IKFGLDGGRIESIMVSAPPLIAWKYNVVPQPGSPEEEMLKVLQQPREWA
Ferrochelatase from Chlamydomonas reinhardtii nucleic acid sequence (SEQ ID NO: 7):
atggcgtcgtttggattgatgcaaaggacggtgcactgtccccagcttgtggaggagcggtgttcgccggtcgct
ggctgctctggtcgtggcctgccagttatccagcggcaacggcgtggcgtgtgcagtgccaccaacggtgtccag
cgagggcgtgtgctgcgccggacggccgcttcgaccgacgtggtctccttcgtggaccccaatgacattagaaaa
cccgcagcagcagcagctggccctgcggtggataaggtcggcgttctgctgttaaaccttggcgggcccgaaaag
ctcgacgacgtcaagcctttcctgtataacctattcgccgacccagaaattattcgcctgccagcggcagctcag
ttcctgcagccgctgctcgcgacgatcatctccacgcttcgcgccccgaagagcgcggagggctatgaggccatt
ggcggtggtagcccgttgcgtaggattacagacgagcaggcggaggcgctggcggagtctctgcgcgccaagggc
caacctgcgaacgtgtacgtgggcatgcgctattggcacccctacacggaggaggcgctggagcacattaaggcc
gacggcgtcacgcgcctggtcatcctcccgctgtaccctcagttctccatctctaccagcggctccagccttcga
ctgcttgagtcgctcttcaagagcgacatcgcgctcaagtcgctgcggcacacggtcatcccgtcctggtaccag
cggcggggctacgtgagcgcgatggcggacctgattgtagaggagctgaagaagttccgggacgtgcccagcgtg
gagctgtttttctccgcgcacggcgtgcccaagtcctacgtggaggaggcgggcgacccatacaaggaggagatg
gaggagtgcgtgcggctcattacggacgaggtcaagcggcgcggcttcgccaacacgcacacgctggcctaccag
agccgcgtgggccccgcggaatggctcaagccgtacacggatgagtccatcaaggagctgggcaagcgcggcgtc
aagtcgctgctggcggtgcccatcagctttgtcagcgagcacattgagacgttggaggagatcgacatggagtac
cgcgagctggcggaggagagcggcatccgcaactggggccgcgtgccggcgctgaacaccaacgccgccttcatc
gacgacctggcggacgcggtgatggaggcgctgccctacgtgggctgcctggccgggccgacagactcgctggtg
ccgctgggcgacctggagatgctgctgcaggcctacgaccgcgagcgccgcacgctgccgtcaccggtggtgatg
tgggagtggggctggaccaagagcgcggagacgtggaacggccgcattgccatgattgccatcatcatcatcctg
gcgctggaggcagccagcggccagtccatcctcaaaaacctgttcctggcggagtag
Ferrochelatase from Chlamydomonas reinhardtii amino acid sequence (SEQ ID NO: 8):
MASFGLMQRTVHCPQLVEERCSPVAGCSGRGLPVIQRQRRGVCSATNGVQRGRVLRRTAASTDVVSFVDPNDIRK
PAAAAAGPAVDKVGVLLLNLGGPEKLDDVKPFLYNLFADPEIIRLPAAAQFLQPLLATIISTLRAPKSAEGYEAI
GGGSPLRRITDEQAEALAESLRAKGQPANVYVGMRYWHPYTEEALEHIKADGVTRLVILPLYPQFSISTSGSSLR
LLESLFKSDIALKSLRHTVIPSWYQRRGYVSAMADLIVEELKKFRDVPSVELFFSAHGVPKSYVEEAGDPYKEEM
EECVRLITDEVKRRGFANTHTLAYQSRVGPAEWLKPYTDESIKELGKRGVKSLLAVPISFVSEHIETLEEIDMEY
RELAEESGIRNWGRVPALNTNAAFIDDLADAVMEALPYVGCLAGPTDSLVPLGDLEMLLQAYDRERRTLPSPVVW
EWGWTKSAETWNGRIAMIAIIIILALEAASGQSILKNLFLAE
Glutamate-1-semialdehyde aminotransferase (GSA) nucleic acid sequence (SEQ ID NO:
9):
atgcagatgcagctgaacgccaagaccgtgcagggcgccttcaaggcgcagcgccctcgctctgtccgcggcaac
gtggcggtgcgcgcagtggccgctccccctaagctggtcaccaagcgctccgaggagatcttcaaggaggctcag
gagctgctgcccggtggcgtgaactcgcccgtgcgcgctttccgctcggttggtggcggccccatcgtcttcgac
agggtcaagggtgcctactgctgggacgtcgatggcaacaagtacatcgactacgttggctcttggggccctgcc
atttgcggccacggcaacgacgaggtcaacaacgccctgaaggcgcagatcgacaagggcacctcgttcggtgct
ccctgcgagctggagaacgtgctggccaagatggtgattgaccgcgtgccctcggtggagatggtgcgcttcgtg
tcctcgggcactgaggcgtgcctgtcggtgctgcgcctgatgcgcgcatacaccggccgcgagaaggtgctgaag
ttcaccggctgctaccacggccacgccgactccttcctggtgaaggccggctccggtgtgatcaccctgggcctg
cccgactcgcccggtgtgcccaagagcaccgccgccgccaccctgaccgccacctacaacaacctggactccgtg
cgcgagctgttcgccgccaacaagggcgagattgccggtgtgatcctggagcccgtggtcggcaacagcggcttc
attgtgcccaccaaggagttcctgcagggcctgcgcgagatctgcacggctgagggcgccgtgctgtgcttcgat
gaggtcatgaccggcttccgcattgccaagggctgcgcccaggagcacttcggtatcacccccgacctgaccacc
atgggcaaggtcattggtggcggcatgcctgtgggcgcctacggcggcaagaaggagatcatgaagatggtcgcc
cccgccggccccatgtaccaggccggcaccctttcgggcaaccccatggccatgactgccggcatcaagacgctg
gagatcctgggccgccccggcgcctacgagcacctggagaaggtgaccaagcgcctgatcgacggcatcatggcc
gccgccaaggagcacagccacgagatcaccggcggcaacatcagcggcatgtttggcttcttcttctgcaagggc
cctgtgacctgcttcgaggacgccctggcggccgacactgccaagttcgcgcgcttccaccgcggcatgctggag
gagggcgtctacctggctccctcgcagttcgaggccggcttcacctctctggcccactccgaggcggacgtggat
gccacgatcgccgccgctcgccgcgtgttcgcccgcatctaa
Glutamate-1-semialdehyde aminotransferase (GSA) amino acid sequence (SEQ ID NO:
10):
MQMQLNAKTVQGAFKAQRPRSVRGNVAVRAVAAPPKLVTKRSEEIFKEAQELLPGGVNSPVRAFRSVGGGPIVFD
RVKGAYCWDVDGNKYIDYVGSWGPAICGHGNDEVNNALKAQIDKGTSFGAPCELENVLAKMVIDRVPSVEMVRFV
SSGTEACLSVLRLMRAYTGREKVLKFTGCYHGHADSFLVKAGSGVITLGLPDSPGVPKSTAAATLTATYNNLDSV
RELFAANKGEIAGVILEPVVGNSGFIVPTKEFLQGLREICTAEGAVLCFDEVMTGFRIAKGCAQEHFGITPDLTT
MGKVIGGGMPVGAYGGKKEIMKMVAPAGPMYQAGTLSGNPMAMTAGIKTLEILGRPGAYEHLEKVTKRLIDGIMA
AAKEHSHEITGGNISGMFGEFECKGPVTCFEDALAADTAKFAREHRGMLEEGVYLAPSQFEAGFTSLAHSEADVD
ATIAAARRVFARI
glutamyl-trna reductase (HEMA) nucleic acid sequence (SEQ ID NO: 11):
atgcagaccactatgcagcagcgtctccagggccgtaacgtggccgggcggagcgtcgctccctcggtccctgcc
catcgctccttccactcacaccgggctgccactcaaaccgctacgatcagcgctgctgctagctcaaccaccaag
ctgccagcttcgcatctggagagcagcaagaaggcgctggattcgctgaagcagcaggccgtcaatcgctacgcg
ggtgacaagaagagctccattattgccattggtctcaccattcacaacgcacccgtggagctgcgcgagaagctg
gctgtgcctgaggctgaatggccgcgtgctattgaggagctctgccagttcccgcacatcgaggaggccgcggtg
ctgtcgacgtgcaatcgcatggagctctacgttgtcggtctgtcgtggcaccgcggcgttcgcgaggtggaggag
tggctgtctcgcaccagcggcgtgcctctggatgagctgcgcccctacctgttcctgctgcgcgaccgcgacgcc
acgcaccacctgatgcgcgtgtcgggtggccttgactcgctggttatgggcgagggccagattctcgcccaagtg
cgccaggtctacaaggtcggccagaactgccccggcttcggtcgccacctgaacggcctgttcaagcaggctatc
accgctggcaagcgcgtgcgtgccgagacctccatctccaccggctccgtctccgtctcatccgccgccgtcgag
ctggcgcagctcaagctccccacccacaactggtccgacgctaaggtctgcatcatcggcgctggcaagatgtct
acgctgctggtgaagcacctgcagagcaagggctgcaaggaggtgacggtgctcaaccgctctctgccgcgcgcc
caggcgctggcggaggagttccctgaggtcaagttcaacatccacctgatgcccgacctgctgcagtgcgtggag
gccagcgacgtcatcttcgccgcctccggctctgaggagatcctcatccacaaggagcatgtcgaggccatgtcc
aagccatcggacgttgttggctccaagcgccgcttcgtcgacatctccgtgccccgcaacatcgcccccgccatc
aacgagctggagcacggcatcgtctacaacgtcgacgacctgaaggaggttgtggccgccaacaaggagggccgc
gcgcaggcggccgccgaggccgaggtgctgatccgcgaggagcagcgcgcgttcgaggcctggcgtgactctctg
gagaccgtgcccaccatcaaggcgctgcgctccaaggccgagaccatccgcgccgccgagtttgagaaggccgtg
tctcgcctgggcgaggggctatccaagaagcagctcaaggcggtggaggagctcagcaagggcatcgtcaacaag
ctgctgcacgggcccatgacggcactgcgctgcgacggcaccgatccggatgccgtgggccagaccctcgcgaac
atggaggccctggagcgcatgttccagctctcggaggtggacgtggccgcgctggcgggcaagcagtaa
glutamyl-trna reductase (HEMA) amino acid sequence (SEQ ID NO: 12):
MQTTMQQRLQGRNVAGRSVAPSVPAHRSFHSHRAATQTATISAAASSTTKLPASHLESSKKALDSLKQQAVNRYA
GDKKSSIIAIGLTIHNAPVELREKLAVPEAEWPRAIEELCQFPHIEEAAVLSTCNRMELYVVGLSWHRGVREVEE
WLSRTSGVPLDELRPYLFLLRDRDATHHLMRVSGGLDSLVMGEGQILAQVRQVYKVGQNCPGFGRHLNGLFKQAI
TAGKRVRAETSISTGSVSVSSAAVELAQLKLPTHNWSDAKVCIIGAGKMSTLLVKHLQSKGCKEVTVLNRSLPRA
QALAEEFPEVKFNIHLMPDLLQCVEASDVIFAASGSEEILIHKEHVEAMSKPSDVVGSKRRFVDISVPRNIAPAI
NELEHGIVYNVDDLKEVVAANKEGRAQAAAEAEVLIREEQRAFEAWRDSLETVPTIKALRSKAETIRAAEFEKAV
SRLGEGLSKKQLKAVEELSKGIVNKLLHGPMTALRCDGTDPDAVGQTLANMEALERMFQLSEVDVAALAGKQ
Light independent protochlorophyllide reductase subunit N (ch1N) nucleic acid
sequence (SEQ ID NO: 13):
atgttatactcacaatttaaacattcggtgcctttaggccgtaagtctccccttctttcagggggccccccttct
gggggtcgcccaacaacggctgcctcaggcctaggtcgcaacgtggccgtaagaattgggaccccgttgggcttt
gcccttcgggcccaggtaattatggcagctgcgggcaatactagcggtgcgccgcaccccgtaggggagtcccag
cctgcgttgtcccaggtggattctcaacttgtaattgagtgtgaaacaggaaattaccatactttttgcccaatt
agttgtgtttcttggttataccaaaaaattgaagatagttttttcttagttattggtacaaaaacgtgtgggtat
tttttacaaaatgctttaggggttatgatttttgccgaacctcgttacgctatggcggaattagaagaaagcgat
atttcggcgcaattaaatgattacaaagaattaaaacgtctatgtttacaaattaaacaagaccgtaacccaagt
gttattgtgtggattggcacatgcacaaccgaaattattaaaatggatttagaaggtatggcaccgaaactagaa
gctgaaatcggtattccaattgtggtagcacgcgcaaatggacttgattatgcttttacacaaggtgaagatact
gttttagctgcgatggtccaaaaatgcccggaattaggcgctattccagctattgtacctcagattccttctgac
tctcgtacacttagccaactatctgtagcggcttcggtacccgaaaacagtgcgtctgggccagaaggggagcct
tcactagcccagaagggaatggattctaagttaacaaacaactctccatgccgagtagattctgtctcagaatct
accccggcgtttcctggacgtgctccgcacgtcgggaaaagtactcctcaaaatttagttttatttggttcatta
cctagcacgatggcaaatcaactggagtttgaattaaaacgccaaggtattaatgttactgggtggttacctgcg
gctcgctattcatctttacctgcattaggtgaaaacgtgtatgtttgtgggattaatccatttttaagtcgaact
gctacttctttaatgcgtcgtcgtaaatgcaaattaatttcagctcctttcccaattggtccagatggtacaaaa
gcttgggtcgaaaaaatttgtaatgttttcggtgttacaccaactggtttagaagatcgtgaacgtcttgtttgg
gaaggtttaaaagattatttaaatttcgtaaaagggaaatctgttttctttatgggtgataatctgttagaaatt
tcattagcccgttttttaattcgctgtggtatgaccgtttatgaaatcggtattccgtacatggaccaacgattt
caagctggggaattagaattattaaaaaaaacatgcatggaaatgaacgtgcccctaccgcgtattgttgaaaaa
cctgataattactatcaaattcaacgtattaaagaattacaaccagatttagttattaccggcatggcccatgca
aacccactggaagcgcgcggcattactacgaaatggtccgttgaatttacgtttgcgcaaattcatgggtttggc
aacgcacgtgatatcttagaattagttacaaaaccgttacgtcgtaataaaaatctatctaaatatcaatttccg
ttagatagctgggacaagcctgcttccgtaggcgctcacgaactgtcggcctaa
Light independent protochlorophyllide reductase subunit N (ch1N) amino acid sequence
(SEQ ID NO: 14):
MLYSQFKHSVPLGRKSPLLSGGPPSGGRPTTAASGLGRNVAVRIGTPLGFALRAQVIMAAAGNTSGAPHPVGESQ
PALSQVDSQLVIECETGNYHTFCPISCVSWLYQKIEDSFFLVIGTKTCGYFLQNALGVMIFAEPRYAMAELEESD
ISAQLNDYKELKRLCLQIKQDRNPSVIVWIGTCTTEIIKMDLEGMAPKLEAEIGIPIVVARANGLDYAFTQGEDT
VLAAMVQKCPELGAIPAIVPQIPSDSRTLSQLSVAASVPENSASGPEGEPSLAQKGMDSKLTNNSPCRVDSVSES
TPAFPGRAPHVGKSTPQNLVLFGSLPSTMANQLEFELKRQGINVTGWLPAARYSSLPALGENVYVCGINPFLSRT
ATSLMRRRKCKLISAPFPIGPDGTKAWVEKICNVFGVTPTGLEDRERLVWEGLKDYLNFVKGKSVFFMGDNLLEI
SLARFLIRCGMTVYEIGIPYMDQRFQAGELELLKKTCMEMNVPLPRIVEKPDNYYQIQRIKELQPDLVITGMAHA
NPLEARGITTKWSVEFTFAQIHGFGNARDILELVTKPLRRNKNLSKYQFPLDSWDKPASVGAHELSA
Light Independent protochlorophyllide subunit B (ch1B) nucleic acid sequence (SEQ ID
NO: 15):
atgaaattagcgtattggatgtatgcgggaccggctcatattggaacattacgagttgcaagctcgtttcgaaat
gtgcatgctattatgcatgctcccttaggcgatgattattttaacgtaatgcgttcaatgttagaacgtgaacgt
gattttacgccagtgacggcaagtattgttgatcgtcatgttttagctcgtggttcacaagaaaaagttgttgaa
aacattcaacgaaaagataaagaagaatgtccggatttaattttattaacaccaacatgtacctcaagtattttg
caagaagatttacaaaattttgtaaatcgcgcggccgaagtagcaaagcgttcggatgttttattagctgacgtt
aaccattaccgagtgaatgaattacaagcggctgaccgtacgttagagcaaattgtacgcttttatttagaaaaa
gaagtaaataaacttcacgcggagttaggcggccttaaaaaaccgcttcgctttgcccagcgtacccaaaagccg
tctgccaatattttaggcatgtttacactaggtttccataatcaacatgactgtcgtgaattaaaacgtttatta
aatgatttaggtatcgaagtcaatgaagtgattcctgaaggtagttttgtacatggattaaaaaatttaccaaaa
gcgtggtttaacatcgtcccgtatcgtgaagttggtttaatgacggcaatttatttagaaaaagaatttggcatg
ccttatacctcaatcacgccaatgggcattattgacaccgcggcgtttattcgtgaaattgcggccatttgtagt
caaattagcacttcacaggcatctacaaactcaactgaaggactccagaggggagaaaatgtcagtttaactgaa
actaattcgattatttttaataaagcaaaatatgaacaatacattaatcaacaaacgcattttgtttctcaagca
gcttggttttcacgttctattgactgtcaaaatttaaccggtaaaaaaaccgttgtgtttggtgatgcaactcac
gcggcaagtatgacgaaaattcttgtgcgcgaaatgggtattcatgttgtttgcgcgggcacgtattgtaaacat
gatgcagattggtttagagagcaagtttcaggtttttgtgatcaagttttaattacagatgatcacagccaaatt
gcggaaatcattgctcaaattgaacctgcagccatttttggtacacaaatggaacgtcatgttgggaaaaggtta
gatattccttgtggggttatttctgcaccggtacatattcaaaacttcccactaggctttagaccgtttttaggg
tatgaaggtactaatcaaatttccgatttagtttataattcgtttagtttaggtatggaagatcacttactagaa
attttcaacggtcatgacaataaagaagttattacacgttcgtattcttcagaaactgatttagaatggacaaaa
gaagcattagatgaactagctcgtgttcctggttttgttcgttcaaaagttaaacgtaatactgaaaaatttgcg
cgtacaaataaaaatcaagttattactattgaagttatgtacgcagctaaagaagcggtatcagcgtaa
Light Independent protochlorophyllide subunit B (ch1B) amino acid sequence (SEQ ID
NO: 16):
MKLAYWMYAGPAHIGTLRVASSFRNVHAIMHAPLGDDYFNVMRSMLERERDFTPVTASIVDRHVLARGSQEKVVE
NIQRKDKEECPDLILLTPTCTSSILQEDLQNFVNRAAEVAKRSDVLLADVNHYRVNELQAADRTLEQIVRFYLEK
EVNKLHAELGGLKKPLRFAQRTQKPSANILGMFTLGFHNQHDCRELKRLLNDLGIEVNEVIPEGSFVHGLKNLPK
AWFNIVPYREVGLMTAIYLEKEFGMPYTSITPMGIIDTAAFIREIAAICSQISTSQASTNSTEGLQRGENVSLTE
TNSIIFNKAKYEQYINQQTHFVSQAAWFSRSIDCQNLTGKKTVVFGDATHAASMTKILVREMGIHVVCAGTYCKH
DADWFREQVSGFCDQVLITDDHSQIAEIIAQIEPAAIFGTQMERHVGKRLDIPCGVISAPVHIQNFPLGFRPFLG
YEGTNQISDLVYNSFSLGMEDHLLEIFNGHDNKEVITRSYSSETDLEWTKEALDELARVPGFVRSKVKRNTEKFA
RTNKNQVITIEVMYAAKEAVSA
Light independent protochlorophyllide reductase subunit L (ch1L) nucleic acid
sequence (SEQ ID NO: 17):
atgaaattagcagtttatggcaaaggtggtattggtaaatccacaacaagttgtaacatttcaattgcattagca
aaacgtggcaaaaaagtattacaaattggttgtgatccaaaacacgatagtacttttacattaaccggtttttta
attccaacaattattgatactttacaaagtaaagattatcattacgaagatgtttggccggaagatgttatttac
caaggctacgggagtgtggattgtgttgaagcaggtggcccgccagccggcgccggctgtggtgggtatgttgtt
ggtgaaacagttaaattattaaaagaattaaatgcattttatgaatatgatgttattctgtttgatgttttaggg
gatgttgtatgtggtgggtttgctgcacctttaaattacgccgactattgcattattgtcacagataatggcttt
gatgcgttatttgccgcaaaccgtattgctgcttcagtgcgcgaaaaagcgcgcattcacccattacgtttagct
gggttaattgggaatcgtacagccaaacgcgatttaatcgataaatacgttgaagcgtgcccgatgccagtctta
gaggtattaccgttaattgaagacattcgtgtgtcacgcgtaaaaggtaaaacattatttgaaatggcagaacat
gattcatcattacactacatttgtgacttttatttaaatattgcggatcaattattaactgaaccagaaggtgtt
gttccgcgcgaattagcagaccgtgaattatttactctattatcagatttctatttaaacgctgggactcctagc
cctagtggatctgagttcggctcaggcgcccttagcggaacgagcggcgaaacagctcccggtaatatgggtcag
cacatgagtaacgcagtaaaaacaaacgaacaggaaatgaatttctttcttgtgtaa
Light independent protochlorophyllide reductase subunit L (ch1L) amino acid sequence
(SEQ ID NO: 18):
MKLAVYGKGGIGKSTTSCNISIALAKRGKKVLQIGCDPKHDSTFTLTGFLIPTIIDTLQSKDYHYEDVWPEDVIY
QGYGSVDCVEAGGPPAGAGCGGYVVGETVKLLKELNAFYEYDVILFDVLGDVVCGGFAAPLNYADYCIIVTDNGF
DALFAANRIAASVREKARIHPLRLAGLIGNRTAKRDLIDKYVEACPMPVLEVLPLIEDIRVSRVKGKTLFEMAEH
DSSLHYICDFYLNIADQLLTEPEGVVPRELADRELFTLLSDFYLNAGTPSPSGSEFGSGALSGTSGETAPGNMGQ
HMSNAVKTNEQEMNFFLV
Magnesium Chelatase subunit H (CHLH2) nucleic acid sequence (SEQ ID NO: 19):
atgcggattgtgctggtcagcggcttcgagagctttaacgtgggcctgtacaaggatgcggcggagctgctgaag
cgctccatgcccaacgtcacactccaggtgttctccgaccgcgacctggcctccgacgccacccgctcccggctg
gaggcggctctggggcgcgccgacatcttcttcggatcactgctgttcgactacgaccaggtggagtggctacgg
gcccggctggagcgggtgcctgtgcggctagtgtttgagtcggcgttggagctcatgagctgcaacaaggtgggg
tcgttcatgatgggcggcggcggtcccggcggcggcccgcccggcaaggcgcccggcccgccgcccgcggtgaag
aaggttctctccatgtttggaagcggtcgcgaggaggacaagatgggcggctcctccaatgtggtggccatgttc
agttacctggtggagaccctgatggagccaacgggtgggttatttggtagttggtggttgtgttatggttggccg
tttcggttgggtgatctgggctggtatctacaacccccctcaaccctcacgcctccaggctacgtgccgccgcct
gtggtggagactcccgcactgggctgcctccacccctccgcgcccggccgctacttcgagtcccccgccgagtac
atgaagtggtacgccagggagggcccgctgcgcggcacgggcgccccggtggttggcgtgctgctgtaccgcaag
catgtgatcaccgaccagccgtacatcccgcagctggtcagccagctggaggcggaggggctgctgcccgtgccc
atcttcatcaacggcgtggaggcgcacaccgtggttcgcgacctgctgacctccgtgcacgagcaggatctgctt
gcacgcggcgagacgggcgccatcagccccaccctgaagcgggacgcggtcaaggtggacgcggtggtgagcacc
attggcttcccgctggtgggcggccccgccggcaccatggagggcgggcggcaggcggaggtggccaaggccatc
ctgggcgccaaggacgtgccgtacacggtggcggcgccgctgcttattcaggacatggagagctggagcagggac
ggcgtggcgggtctccagagtgtggtgctgtactcgctgccggagctggacggcgcagtggacacggtgccactg
ggggggctggtgggggacgacatctacctggtgccggagcgggtgaagaagctggcggggcggctcaagtcgtgg
cgtacgacacgcactaagcatgcctctgtttgtgacgtccagcccctcccccccccgtctcccctctccaccctc
cctctcccttcctctcccttcctctcactctccaccctcttccccctccgcccaaacataacgaggcgggggctg
ctgggcgcaagcgggccctggagtacccgctgcgacctagctagtccaactccacccatcccccaatgccgcaat
agctttccggagatgagcacacacacacacacacacacacacacacacacacacacacacacacacacacacaca
cgccacccacgcacacacacacacacacacgctccccccgctcgccacacccccatcccaccccacccgcaggag
ctgctgacgtaccccgcggactggggcccggccgagtggggcccgctgccctacctgcccgaccccgacgtgctg
gttcgccgcatggaggcgcagtggggcgagctgcgagcctaccgcggcctcaacacctcggcgcgcggcatgttc
caggagtacggggctgacgtggtcctgcacttcggcatgcacggcaccgtggagtggttgcctggggcgccgctg
gggaacaacggcctcagctggagcgacgtgctgctcggcgagctgccaaacgtgtacgtgtacgctgccaacaac
ccctccgagtccatcgtggcaaagcggcgcggctacggcaccatcgtcagccacaacgtgccgccgtacgggcgg
gcgggtctgtacaagcagctttccagcctcaaggagacgcttcaggagtaccgcgaggccgcgcaggccgcacgt
gcccgagcaggagccagcagcagcagcggcagtagcagcagtagcagtagcagcggcagtggcagtagcagcagc
agtgtggagctgcgggcggcgttggcaccggtgttcgacgcctacactgaccgcctgtatgcctacctgcagctg
ctggaggggcggctgttcagcgaggggctacacgtactgggagcgccgccggcgccgccgcaggtgggtggtttt
cccgcgagcttccaacggtaccgtaaactgcccaactgcccaacttctccccaaacacaggaggctgtcaagatc
cggaacctgctcatgcagaacacgcaggagctggacgggctgctcaagggcctgggtgggcgttacgtgcttccc
gaggcgggcggcgacctgctgcgggacgggtcgggcgtgctgcccaccggccgcaacatccacgcactggacccc
taccgcatgccctcccccgccgccatggcccgtggggcggcggtggcggcggccattcttgagcagcaccgggcg
gctaacagcggggcgtggcccgagacctgcgccgtcaacctgtgggggctggactccatcaagagcaagggcgag
agtgtgggggtggtgctggcgctggtgggggcggtgccggtgcgcgagggtacgggccgcgtcgcgcgcttccaa
ctggtgccgctgtcagagttgggccggccgcgtgtggacgtgctttgtaacatgagcggcatcttccgcgactcc
ttccagaacgtggtggagctgctcgacgacctgtttgcaagggccgccgccgccgctgacgagccagatgacatg
aacttcatcgccaaacacgcccgagccatggagaagcagggcctgtccgccacctcggcccgcctgttctccaac
ccggctggcgactacgggtcgatggtcaacgagcgagtggggcagggcagctgggccaacggcgacgagctgggt
gacacgtgggcggcccgcaacgccttcagctacggccgaggcaaggagcgaggcacggcgcggcccgaggtgctg
caggcgctgctcaagaccacggaccggatcgtgcagcagatcgacagtgtggagtacggcctgacagacatccag
gagtactacgccaacacgggcgccctcaagagagccgccgaggtggccaaaggcgacccgggccccggtggccgg
cggccgcgcgtggggtgttccattgtggaggcctttggcggcgcgggcgcgggcgcgggcggcgccggtggagcg
ggcgtgccgccgcctcgcgagctggaggaggtgctgcgcctggagtaccgctcgaagctgctcaaccccaagtgg
gcccgggccatggcggcgcagggcagcggcggcgcctacgagatcagtcagcgcatgacggcgttggtgggctgg
ggcgccaccaccgatttcagggagggctgggtgtgggacccaggcgccatggacacgtatgtgggcgatgaggag
atggccagcaagctcaagaagaacaacccgcaggcctttgccaacgtgctgcggcgcatgctggaggcggcgggc
cgcggcatgtggagccccaacaaggaccagctggcacagctcaagtcgctgtacagcgagatggacgaccagctg
gagggggtgacg
Magnesium Chelatase subunit H (CHLH2) amino acid sequence (SEQ ID NO: 20):
MRIVLVSGFESFNVGLYKDAAELLKRSMPNVTLQVFSDRDLASDATRSRLEAALGRADIFFGSLLFDYDQVEWLR
ARLERVPVRLVFESALELMSCNKVGSFMMGGGGPGGGPPGKAPGPPPAVKKVLSMFGSGREEDKMGGSSNVVAMF
SYLVETLMEPTGGLFGSWWLCYGWPFRLGDLGWYLQPPSTLTPPGYVPPPVVETPALGCLHPSAPGRYFESPAEY
MKWYAREGPLRGTGAPVVGVLLYRKHVITDQPYIPQLVSQLEAEGLLPVPIFINGVEAHTVVRDLLTSVHEQDLL
ARGETGAISPTLKRDAVKVDAVVSTIGFPLVGGPAGTMEGGRQAEVAKAILGAKDVPYTVAAPLLIQDMESWSRD
GVAGLQSVVLYSLPELDGAVDTVPLGGLVGDDIYLVPERVKKLAGRLKSWRTTRTKHASVCDVQPLPPPSPLSTL
PLPSSPFLSLSTLFPLRPNITRRGLLGASGPWSTRCDLASPTPPIPQCRNSFPEMSTHTHTHTHTHTHTHTHTHT
RHPRTHTHTHAPPARHTPIPPHPQELLTYPADWGPAEWGPLPYLPDPDVLVRRMEAQWGELRAYRGLNTSARGMF
QEYGADVVLHFGMHGTVEWLPGAPLGNNGLSWSDVLLGELPNVYVYAANNPSESIVAKRRGYGTIVSHNVPPYGR
AGLYKQLSSLKETLQEYREAAQAARARAGASSSSGSSSSSSSSGSGSSSSSVELRAALAPVFDAYTDRLYAYLQL
LEGRLFSEGLHVLGAPPAPPQVGGFPASFQRYRKLPNCPTSPQTQEAVKIRNLLMQNTQELDGLLKGLGGRYVLP
EAGGDLLRDGSGVLPTGRNIHALDPYRMPSPAAMARGAAVAAAILEQHRAANSGAWPETCAVNLWGLDSIKSKGE
SVGVVLALVGAVPVREGTGRVARFQLVPLSELGRPRVDVLCNMSGIFRDSFQNVVELLDDLFARAAAAADEPDDM
NFIAKHARAMEKQGLSATSARLFSNPAGDYGSMVNERVGQGSWANGDELGDTWAARNAFSYGRGKERGTARPEVL
QALLKTTDRIVQQIDSVEYGLTDIQEYYANTGALKRAAEVAKGDPGPGGRRPRVGCSIVEAFGGAGAGAGGAGGA
GVPPPRELEEVLRLEYRSKLLNPKWARAMAAQGSGGAYEISQRMTALVGWGATTDFREGWVWDPGAMDTYVGDEE
MASKLKKNNPQAFANVLRRMLEAAGRGMWSPNKDQLAQLKSLYSEMDDQLEGVT
Magnesium Chelatase subunit 1 (CHLI1) Chlamydomonas reinhardtii nucleic acid
sequence (SEQ ID NO: 21):
atggccctgaacatgcgtgtttcctcttccaaggtcgctgccaagcagcagggccgcatctccgcggtgccggtt
gtgtcgagcaaggtggcctcctccgcccgcgtggcccccttccagggcgctcccgtggccgcgcagcgcgctgct
ctgctggtgcgcgccgctgccgctactgaggtcaaggctgctgagggccgcactgagaaggagctgggccaggcc
cgccccatcttccccttcaccgccatcgtgggccaggatgagatgaagctggcgctgattctgaacgtgatcgac
cccaagatcggtggtgtcatgatcatgggcgaccgtggcactggcaagtccaccaccattcgtgccctggcggat
ctgctgcccgagatgcaggtggttgccaacgacccctttaactcggaccccaccgaccccgagctgatgagcgag
gaggtgcgcaaccgcgtcaaggccggcgagcagctgcccgtgtcttccaagaagattcccatggtggacctgccc
ctgggcgccactgaggaccgcgtgtgcggcaccatcgacatcgagaaggcgctgaccgagggtgtcaaggcgttc
gagcccggcctgctggccaaggccaaccgcggcatcctgtacgtggatgaggtcaacctgctggacgaccacctg
gtcgatgtgctgctggactcggccgcctccggctggaacaccgtggagcgcgagggtatctccatcagccacccc
gcccgcttcatcctggtcggctcgggcaaccccgaggagggtgagctgcgcccccagctgctggatcgcttcggc
atgcacgcccagatcggcaccgtcaaggacccccgcctgcgtgtgcagatcgtgtcgcagcgctcgaccttcgac
gagaaccccgccgccttccgcaaggactacgaggccggccagatggcgctgacccagcgcatcgtggacgcgcgc
aagctgctgaagcagggcgaggtcaactacgacttccgcgtcaagatcagccagatctgctcggacctgaacgtg
gacggcatccgcggcgacatcgtgaccaaccgcgccgccaaggccctggccgccttcgagggccgcaccgaggtg
acccccgaggacatctaccgtgtcattcccctgtgcctgcgccaccgcctccggaaagaccccctggctgagatc
gacgacggtgaccgcgtgcgtgagatcttcaagcaggtgttcggcatggagtaa
Magnesium Chelatase subunit 1 (CHLI1) Chlamydomonas reinhardtii amino acid
sequence (SEQ ID NO: 22):
MALNMRVSSSKVAAKQQGRISAVPVVSSKVASSARVAPFQGAPVAAQRAALLVRAAAATEVKAAEGRTEKELGQA
RPIFPFTAIVGQDEMKLALILNVIDPKIGGVMIMGDRGTGKSTTIRALADLLPEMQVVANDPFNSDPTDPELMSE
EVRNRVKAGEQLPVSSKKIPMVDLPLGATEDRVCGTIDIEKALTEGVKAFEPGLLAKANRGILYVDEVNLLDDHL
VDVLLDSAASGWNTVEREGISISHPARFILVGSGNPEEGELRPQLLDRFGMHAQIGTVKDPRLRVQIVSQRSTED
ENPAAFRKDYEAGQMALTQRIVDARKLLKQGEVNYDFRVKISQICSDLNVDGIRGDIVTNRAAKALAAFEGRTEV
TPEDIYRVIPLCLRHRLRKDPLAEIDDGDRVREIFKQVFGME
Magnesium Chelatase sunubit1 (CHLI2) Chlamydomonas reinhardtii nucleic acid
sequence (SEQ ID NO: 23):
atgcagagtctccagggtcagcgcgcgttcactgcggtgcgccagggtcgggcgggtcccctgcggactcgcctg
gtcgtgcgctcgtctgttgccttgccatccacgaaagccgcgaagaagccgaacttcccgttcgtcaagattcag
ggccaggaggagatgaagcttgcactgctgctgaacgtggtcgaccccaacatcggcggagtgcttattatgggt
gaccgcggcactgccaagtcggtcgcggtccgcgccctggtggatatgcttcccgacattgacgtggttgagggc
gacgccttcaacagctcccccaccgaccccaagttcatgggccccgacaccctgcagcgcttccgcaacggcgag
aagctgcccaccgtccgcatgcggacccccctggtggagctgcctctgggcgccaccgaggaccgcatctgcggc
accatcgacatcgagaaggcgctgacgcagggcatcaaggcctacgagcccggcctgctggccaaggccaaccgc
ggcatcctgtatgtggacgaggtgaacctgctggatgatggcctggttgatgtcgtgctggactcgtcggctagc
ggcctgaacactgtggagcgtgagggtgtgtccattgtgcaccctgcccgcttcatcatgattggctcaggcaac
ccccaggagggtgagctgcgcccgcagctgctggatcgcttcggcatgagcgtcaacgtggccacgctgcaggac
accaagcagcgcacgcagctggtgctggaccggcttgcgtacgaggcggaccctgacgcatttgtggactcgtgc
aaggccgagcagacggcgctcacggacaagctggaggcggcccgccagcgcctgcggtccgtcaagatcagcgag
gagctgcagatcctgatctcggacatttgctcgcgcctggatgtggatggcctgcgcggtgacattgtgatcaac
cgcgccgccaaggcgcttgtggccttcgagggccgcaccgaggtgaccacgaatgacgtggagcgcgtcatctcg
ggctgcctcaaccaccgcctgcgcaaggacccgctggaccccattgacaacggcaccaaggtggccatcctgttc
aagcgcatgaccgaccccgagatcatgaagcgcgaggaggaggccaagaagaagcgcgaggaggcggccgccaag
gccaaggcggagggcaaggcggaccgccccacgggcgccaaggctggcgcctgggctggcttgccccctcgtcgg
taa
Magnesium Chelatase sunubit1 (CHLI2) Chlamydomonas reinhardtii amino acid
sequence (SEQ ID NO: 24):
MQSLQGQRAFTAVRQGRAGPLRTRLVVRSSVALPSTKAAKKPNFPFVKIQGQEEMKLALLLNVVDPNIGGVLIMG
DRGTAKSVAVRALVDMLPDIDVVEGDAFNSSPTDPKFMGPDTLQRFRNGEKLPTVRMRTPLVELPLGATEDRICG
TIDIEKALTQGIKAYEPGLLAKANRGILYVDEVNLLDDGLVDVVLDSSASGLNTVEREGVSIVHPARFIMIGSGN
PQEGELRPQLLDREGMSVNVATLQDTKQRTQLVLDRLAYEADPDAFVDSCKAEQTALTDKLEAARQRLRSVKISE
ELQILISDICSRLDVDGLRGDIVINRAAKALVAFEGRTEVTTNDVERVISGCLNHRLRKDPLDPIDNGTKVAILF
KRMTDPEIMKREEEAKKKREEAAAKAKAEGKADRPTGAKAGAWAGLPPRR
Magnesium Chelatase subunit D (CHLD) Chlamydomonas reinhardtii nucleic acid
sequence (SEQ ID NO: 25):
atgaagtctctctgccatgagctcgctggccccagcgttactgggtgcggccggcgaagcctccggaaggctttc
agcggtgccaagattgcgcaggtctctcgccccgctgtgcttaacagcgtgcagcgccaacagcgtctcgcctgt
tctgccgtggccgagctctccgctgctgagctgcgcgccatgaaggtgtctgaggaggactccaagggcttcgat
gcggatgtgtcgacccgcctggcccgctcgtaccctctggcggccgtggtgggccaggacaacatcaagcaggcg
ctgctgctgggcgccgtggacaccgggctgggcggcatcgccatcgccggtcgccgcggtaccgccaagtccatc
atggctcgcggcctgcacgctctgctgccgcccattgaggtggtggagggcagcatctgcaacgccgaccccgag
gacccccgctcctgggaggctggcctggctgagaagtatgcgggcggccctgtgaagaccaagatgcgctcggcg
ccgtttgtgcagatccctctgggtgtgactgaggaccgcttggtgggcactgtggacattgaggcgtccatgaag
gagggcaagactgtgttccagcccggcctgctggctgaggcgcaccgcggcatcctgtacgtggacgagatcaac
ctgctggatgacggcattgccaacctgctgctgtccatcctgtcggacggagtcaacgtggtggagcgcgagggc
atctccatcagccacccctgccggccgctgctgattgccacctacaaccccgaggagggccctctgcgtgagcac
ctgctggaccgcatcgccattggcctcagcgccgacgtccccagcaccagcgacgagcgcgtcaaggccattgac
gcagccatccgcttccaggacaagccgcaggacactattgacgacaccgcggagctcaccgacgccctgcgcacc
tcggtcatcctggctcgcgagtacctgaaggacgtgaccatcgcgccggagcaggtgacctacattgtggaggag
gcgcgccgcggcggagtccaggggcaccgcgcggagctgtacgcggtcaagtgtgccaaggcgtgtgcggctctg
gagggccgtgagcgtgtgaacaaggatgacctgcgccaggccgtgcagctggtcatcctgccgcgcgccaccatc
ctggaccagcccccgcccgagcaggagcagcccccgccgccgcccccgccccctcccccgccgccgccgcaggac
caaatggaggacgaggaccaggaggagaaggaggacgagaaggaggaggaggagaaggagaacgaggaccaggac
gagcccgagatccctcaggagttcatgtttgagtccgagggcgtcatcatggacccctccatcctcatgttcgcg
cagcagcagcagcgcgcgcagggccgctccggccgcgccaagacgctcatcttcagcgacgaccgcggccgctac
atcaagcccatgctgcccaagggtgacaaggtcaagcgcctggcagtggacgccacgcttcgcgccgccgcgccc
taccagaagattcgccggcagcaggccatcagcgagggcaaggtgcagcgcaaggtgtacgtggacaagccagac
atgcgctccaagaagctggcccgcaaggccggtgcgctggtgatttttgttgtggacgcgtccggctccatggct
ctgaaccgcatgagcgccgccaagggcgcctgcatgcgcctgctggctgagtcgtacaccagccgcgaccaggtg
tgcctcatccccttctacggcgacaaggccgaggtgctgctgccgccctccaagtccatcgccatggcccgccgc
cgcctggactcgctgccctgcggcggcggctcgccccttgcgcacggcctgtccacggcggtacgtgtgggcatg
caggccagccaggcgggcgaggtgggccgcgtcatgatggtgctcatcacggacggccgcgccaacgtcagcctg
gccaagtccaacgaggaccccgaggcgctcaagcccgacgcgcccaagcccaccgccgactcgctgaaggacgag
gtgcgcgacatggccaagaaggccgcgtccgccggcatcaacgtgcttgtcattgacacggagaacaagttcgtg
agcaccggctttgcggaggagatctccaaggcagcgcagggcaagtactactacctgcccaacgccagcgacgcc
gccatcgcggcggccgcgtccggcgccatggccgcggccaagggcggctactag
Magnesium Chelatase subunit D (CHLD) Chlamydomonas reinhardtii amino acid
sequence (SEQ ID NO: 26):
MKSLCHELAGPSVTGCGRRSLRKAFSGAKIAQVSRPAVLNSVQRQQRLACSAVAELSAAELRAMKVSEEDSKGFD
ADVSTRLARSYPLAAVVGQDNIKQALLLGAVDTGLGGIAIAGRRGTAKSIMARGLHALLPPIEVVEGSICNADPE
DPRSWEAGLAEKYAGGPVKTKMRSAPFVQIPLGVTEDRLVGTVDIEASMKEGKTVFQPGLLAEAHRGILYVDEIN
LLDDGIANLLLSILSDGVNVVEREGISISHPCRPLLIATYNPEEGPLREHLLDRIAIGLSADVPSTSDERVKAID
AAIRFQDKPQDTIDDTAELTDALRTSVILAREYLKDVTIAPEQVTYIVEEARRGGVQGHRAELYAVKCAKACAAL
EGRERVNKDDLRQAVQLVILPRATILDQPPPEQEQPPPPPPPPPPPPPQDQMEDEDQEEKEDEKEEEEKENEDQD
EPEIPQEFMFESEGVIMDPSILMFAQQQQRAQGRSGRAKTLIFSDDRGRYIKPMLPKGDKVKRLAVDATLRAAAP
YQKIRRQQAISEGKVQRKVYVDKPDMRSKKLARKAGALVIFVVDASGSMALNRMSAAKGACMRLLAESYTSRDQV
CLIPFYGDKAEVLLPPSKSIAMARRRLDSLPCGGGSPLAHGLSTAVRVGMQASQAGEVGRVMMVLITDGRANVSL
AKSNEDPEALKPDAPKPTADSLKDEVRDMAKKAASAGINVLVIDTENKFVSTGFAEEISKAAQGKYYYLPNASDA
AIAAAASGAMAAAKGGY
Magnesium Chelatase subunit H (CHLH1) Chlamydomonas reinhardtii nucleic acid
sequence (SEQ ID NO: 27):
atgcagacttcctcgcttcttggccggcgcacggcccacccggctgcgggcgcgacgcccaagccggttgcgccc
tcgccccgcgtggctagcacccgccaggtcgcgtgcaatgtggcgactggaccccggccgcccatgaccaccttc
accggtggcaacaagggccctgctaagcagcaggtgtcgctggatctgcgcgacgagggcgctggcatgttcacc
agcaccagcccggagatgcgccgtgtcgtccctgacgatgtgaagggtcgcgttaaggtgaaggttgtgtacgtg
gtgctggaggcccagtaccagtcggccatcagcgctgcggtgaagaacatcaacgccaagaactccaaggtgtgc
ttcgaggtggtgggctacctgctggaggagctgcgtgaccagaagaacctcgatatgctcaaggaggatgtggcc
tctgccaacatcttcatcggctcgctcatcttcattgaggagcttgccgagaagattgtggaggcggtgagcccc
ctgcgcgagaagctggacgcgtgcctgatcttcccgtccatgccggcggtcatgaagctgaacaagctgggcacg
ttttcgatggctcagctgggccagtcgaagtcggtgttctcggagttcatcaagtctgctcgcaagaacaacgac
aacttcgaggagggcttgctgaagctggtgcgcaccctgcctaaggtgctgaagtatctgccctcggacaaggcg
caggacgccaagaacttcgtgaacagcctgcagtactggctgggcggtaactcggacaacctggagaacctgctg
ctgaacaccgtcagcaactacgtgcccgctctgaagggcgtggacttcagcgtggctgagcccaccgcctacccc
gatgtgggtatctggcaccctctggcctcgggcatgtacgaggacctgaaggagtacctgaactggtacgacacc
cgcaaggacatggtcttcgccaaggacgcccccgtcattggcctggtgctgcagcgctcgcacctggtgactggc
gatgagggccactacagcggcgtggtcgctgagctggagagccgcggtgctaaggtcatccccgtctttgccggt
ggcctggacttctccgcccccgtcaagaagttcttctacgaccccctgggctctggccgcacgttcgtggacacc
gttgtgtcgctgaccggcttcgcgctggtgggcggccccgcgcgccaggacgcgccgaaggccattgaggcgctg
aagaacctgaacgtgccctacctggtgtcgctgccgctggtgttccagaccactgaggagtggctggacagcgag
ctgggcgtgcaccccgtccaggtggctctgcaggttgccctgcccgagctggatggtgccatggagcccatcgtg
ttcgctggccgtgactcgaacaccggcaagtcgcactcgctgcccgaccgcatcgcttcgctgtgcgctcgcgcc
gtgaactgggccaacctgcgcaagaagcgcaacgccgagaagaagctggccgtcaccgtgttcagcttcccccct
gacaagggcaacgtcggcactgccgcctacctgaacgtgttcggctccatctaccgcgtgctgaagaacctgcag
cgcgagggctacgacgtgggcgccctgccgccctcggaggaggatctgatccagtcggtgctgacccagaaggag
gccaagttcaactcgaccgacctgcacatcgcctacaagatgaaggtggacgagtaccagaagctgtgcccttac
gccgaggcgctggaggagaactggggcaagccccccggcaccctgaacaccaacggccaggagctgctggtgtac
ggccgccagtacggcaacgtcttcatcggcgtgcagcccaccttcggctacgagggcgacccgatgcgcctgctg
ttctcgaagtcggccagcccccaccacggcttcgccgcctactacaccttcctggagaagatcttcaaggccgac
gccgtgctgcacttcggcacccacggctcgctggagttcatgcccggcaagcaggtcggcatgtcgggtgtgtgc
taccccgactcgctgatcggcaccatccccaacctctactactacgccgccaacaacccgtctgaggccaccatc
gccaagcgccgctcgtacgccaacaccatttcgtacctgacgccgcctgccgagaacgccggcctgtacaagggc
ctgaaggagctgaaggagctgatcagctcgtaccagggcatgcgtgagtctggccgcgccgagcagatctgcgcc
accatcattgagaccgccaagctgtgcaacctggaccgcgacgtgaccctgcccgacgctgacgccaaggacctg
accatggacatgcgcgacagcgttgtgggccaggtgtaccgcaagctgatggagattgagtcccgcctgctgccc
tgcggcctgcacgtggtgggctgcccgcccaccgccgaggaggccgtggccaccctggtcaacatcgctgagctg
gaccgcccggacaacaacccccccatcaagggcatgcccggcatcctggcccgcgccattggtcgcgacatcgag
tcgatttacagcggcaacaacaagggcgtcctggctgacgttgaccagctgcagcgcatcaccgaggcctcccgc
acctgcgtgcgcgagttcgtgaaggaccgcaccggcctgaacggccgcatcggcaccaactggatcaccaacctg
ctcaagttcaccggcttctacgtggacccctgggtgcgcggcctgcagaacggcgagttcgccagcgccaaccgc
gaggagctgatcaccctgttcaactacctggagttctgcctgacccaggtggtcaaggacaacgagctgggcgcc
ctggtagaggcgctgaacggccagtacgtcgagcccggccccggcggtgaccccatccgcaaccccaacgtgctg
cccaccggcaagaacatccacgccctggaccctcagtcgattcccactcaggccgcgctgaagagcgcccgcctg
gtggtggaccgcctgctggaccgcgagcgcgacaacaacggcggcaagtaccccgagaccatcgcgctggtgctg
tggggcactgacaacatcaagacctacggcgagtcgctggcccaggtcatgatgatggtcggtgtcaagcccgtg
gccgacgccctgggccgcgtgaacaagctggaggtgatccctctggaggagctgggccgcccccgcgtggacgtg
gttgtcaactgctcgggtgtgttccgcgacctgttcgtgaaccagatgctgctgctggaccgcgccatcaagctg
gcggccgagcaggacgagcccgatgagatgaacttcgtgcgcaagcacgccaagcagcaggcggcggagctgggc
ctgcagagcctgcgcgacgcggccacccgtgtgttctccaacagctcgggctcctactcgtccaacgtcaacctg
gcggtggagaacagcagctggagcgacgagtcgcagctgcaggagatgtacctgaagcgcaagtcgtacgccttc
aactcggaccgccccggcgccggtggcgagatgcagcgcgacgtgttcgagacggccatgaagaccgtggacgtg
accttccagaacctggactcgtccgagatctcgctgaccgatgtgtcgcactacttcgactccgaccccaccaag
ctggtggcgtcgctgcgcaacgacggccgcacccccaacgcctacatcgccgacaccaccaccgccaacgcgcag
gtccgcactctgggtgagaccgtgcgcctggacgcccgcaccaagctgctcaaccccaagtggtacgagggcatg
cttgcctcgggctacgagggcgtgcgcgagatccagaagcgcatgaccaacaccatgggctggtcggccacctcg
ggcatggtggacaactgggtgtacgacgaggccaactcgaccttcatcgaggatgcggccatggccgagcgcctg
atgaacaccaaccccaacagcttccgcaagctggtggccaccttcctggaggccaacggccgcggctactgggac
gccaagcccgagcagctggagcgcctgcgccagctgtacatggacgtggaggacaagattgagggcgtcgaataa
Magnesium Chelatase subunit H (CHLH1) Chlamydomonas reinhardtii amino acid
sequence (SEQ ID NO: 28):
MQTSSLLGRRTAHPAAGATPKPVAPSPRVASTRQVACNVATGPRPPMTTFTGGNKGPAKQQVSLDLRDEGAGMFT
STSPEMRRVVPDDVKGRVKVKVVYVVLEAQYQSAISAAVKNINAKNSKVCFEVVGYLLEELRDQKNLDMLKEDVA
SANTFIGSLIFTEELAEKIVEAVSPLREKLDACLIFPSMPAVMKLNKLGTFSMAQLGQSKSVFSEFIKSARKNND
NFEEGLLKLVRTLPKVLKYLPSDKAQDAKNFVNSLQYWLGGNSDNLENLLLNTVSNYVPALKGVDFSVAEPTAYP
DVGIWHPLASGMYEDLKEYLNWYDTRKDMVFAKDAPVIGLVLQRSHLVTGDEGHYSGVVAELESRGAKVIPVFAG
GLDFSAPVKKFFYDPLGSGRTFVDTVVSLTGFALVGGPARQDAPKAIEALKNLNVPYLVSLPLVFQTTEEWLDSE
LGVHPVQVALQVALPELDGAMEPIVFAGRDSNTGKSHSLPDRIASLCARAVNWANLRKKRNAEKKLAVTVFSFPP
DKGNVGTAAYLNVFGSIYRVLKNLQREGYDVGALPPSEEDLIQSVLTQKEAKFNSTDLHIAYKMKVDEYQKLCPY
AEALEENWGKPPGTLNTNGQELLVYGRQYGNVFIGVQPTFGYEGDPMRLLFSKSASPHHGFAAYYTFLEKIFKAD
AVLHFGTHGSLEFMPGKQVGMSGVCYPDSLIGTIPNLYYYAANNPSEATIAKRRSYANTISYLTPPAENAGLYKG
LKELKELISSYQGMRESGRAEQICATIIETAKLCNLDRDVTLPDADAKDLTMDMRDSVVGQVYRKLMEIESRLLP
CGLHVVGCPPTAEEAVATLVNIAELDRPDNNPPIKGMPGILARAIGRDIESIYSGNNKGVLADVDQLQRITEASR
TCVREFVKDRTGLNGRIGTNWITNLLKFTGFYVDPWVRGLQNGEFASANREELITLFNYLEFCLTQVVKDNELGA
LVEALNGQYVEPGPGGDPIRNPNVLPTGKNIHALDPQSIPTQAALKSARLVVDRLLDRERDNNGGKYPETIALVL
WGTDNIKTYGESLAQVMMMVGVKPVADALGRVNKLEVIPLEELGRPRVDVVVNCSGVFRDLFVNQMLLLDRAIKL
AAEQDEPDEMNFVRKHAKQQAAELGLQSLRDAATRVFSNSSGSYSSNVNLAVENSSWSDESQLQEMYLKRKSYAF
NSDRPGAGGEMQRDVFETAMKTVDVTFQNLDSSEISLTDVSHYFDSDPTKLVASLRNDGRTPNAYIADTTTANAQ
VRTLGETVRLDARTKLLNPKWYEGMLASGYEGVREIQKRMTNTMGWSATSGMVDNWVYDEANSTFIEDAAMAERL
MNTNPNSFRKLVATFLEANGRGYWDAKPEQLERLRQLYMDVEDKIEGVE
Photochlorophyllide reductase subunit B (ch1B) nucleic acid sequence (SEQ ID NO:
29):
atgaaattagcttattggatgtacgcaggtcccgctcatatcggtgtgttgcgtgttagcagctcttttaaaaat
gtacatgccattatgcatgctcctttaggagatgattattttaatgtaatgcgttccatgttagaacgtgaacgt
gattttacaccagtaacagccagtattgtagatcgtcatgttttagcaagaggatcgcaagaaaaagtggttgaa
aatattacgcgaaaaaataaagaagaaactcctgatttaattttattaactcctacttgtacgtcaagcatttta
caagaagatttacacaattttgttgaatcggcattagctaaaccagtacaaatagatgaacatgcagaccataaa
gtaactcaacaaagtgcactttcaagtgtatcccctttactaccgcttgaagaaaatacattaatagtaagtgaa
ctagataagaagcttagcccgtctagcaagttgcatattaatatgcccaatatttgtattcccgaaggagaaggg
gaaggggagcagactaaaaattcaatttttgttaaatctgcaactttaacaaatttgtcagaagaggaactatta
aatcaagaacatcataccaaaacaagaaatcactctgacgttattttagctgatgtaaaccattatcgtgtaaat
gaattacaagctgcagatcgtactcttgaacaaattgtacgttattatatttctcaagcacaaaaacaaaattgt
ttaaacattactaaaacagccaaaccatctgtaaatattattggtatttttactttgggttttcataatcaacat
gattgtcgtgaattaaaacgtttatttaatgatttaggtattcaaatcaatgaaatcatacctgaaggcggaaat
gtacacaacttaaaaaaattaccccaagcttggtttaattttgtgccctaccgtgaaattggcttaatgactgct
atgtatttaaaatccgagtttaatatgccttacgtcgcaattactcctatgggattaattgatacggctgcttgt
attcgttcaatttgtaaaatcattacaactcaattattaaatcagacggctacagtgcaggagccatcaaaattt
atttacccgaaggcgacgtcattagaacaaaccaatattctcgaaacctctcaaaaagaaactattcttaaagac
aatccagatagcggaaataccctttctacaactgtagaagaaattgaaactttatttaataaatatatcgatcaa
caaactcgttttgtttcccaagcagcctggttttcacgttctattgactgtcaaaatttaacaggtaaaaaagcc
gtagttttcggagatgctacacattcagctgccatgacaaaattattagcacgtgaaatgggtattaaggtttca
tgcgctggaacttattgcaaacacgatgcggattggtttagagagcaagttagtgggttttgtgatcaagtttta
attaccgatgatcacacacaagtaggggatatgattgcacaattagaacctgcagccatttttgggacacaaatg
gaacgtcacgttggtaaacgtttagatattccatgtggtgttatatctgctcctgtgcatattcaaaactttccg
ttaggttatcgaccttttttaggttatgaaggtacaaatcaaatagctgatttagtgtataattcatttaatctt
ggaatggaagaccatttattacaaatttttggaggtcatgattcagaaaacaattcgtcaattgcaacgcatttg
aatacaaataacgcaataaatttagcgccaggatatttacctgagggagaaggcagtagtagaacttcaaatgta
gtgtctacaatttctagtgaaaaaaaagccattgtatggtctccagaaggtttagcagaattaaataaagtccca
ggatttgttcgaggaaaagttaaacgtaatacggaaaaatatgctttacaaaaaaattgttcgatgattactgta
gaagttatgtatgcagcaaaagaagctttgtcggcttaa
Photochlorophyllide reductase subunit B (ch1B) amino acid sequence (SEQ ID NO: 30):
MKLAYWMYAGPAHIGVLRVSSSFKNVHAIMHAPLGDDYFNVMRSMLERERDFTPVTASIVDRHVLARGSQEKVVE
NITRKNKEETPDLILLTPTCTSSILQEDLHNFVESALAKPVQIDEHADHKVTQQSALSSVSPLLPLEENTLIVSE
LDKKLSPSSKLHINMPNICIPEGEGEGEQTKNSIFVKSATLTNLSEEELLNQEHHTKTRNHSDVILADVNHYRVN
ELQAADRTLEQIVRYYISQAQKQNCLNITKTAKPSVNIIGIFTLGEHNQHDCRELKRLENDLGIQINEIIPEGGN
VHNLKKLPQAWFNEVPYREIGLMTAMYLKSEFNMPYVAITPMGLIDTAACIRSICKIITTQLLNQTATVQEPSKF
IYPKATSLEQTNILETSQKETILKDNPDSGNTLSTTVEEIETLFNKYIDQQTRFVSQAAWFSRSIDCQNLTGKKA
VVFGDATHSAAMTKLLAREMGIKVSCAGTYCKHDADWFREQVSGFCDQVLITDDHTQVGDMIAQLEPAAIFGTQM
ERHVGKRLDIPCGVISAPVHIQNFPLGYRPFLGYEGTNQIADLVYNSFNLGMEDHLLQIFGGHDSENNSSIATHL
NTNNAINLAPGYLPEGEGSSRTSNVVSTISSEKKAIVWSPEGLAELNKVPGFVRGKVKRNTEKYALQKNCSMITV
EVMYAAKEALSA
Photochlorophyllide reductase subunit L (chIL) nucleic acid sequence (SEQ ID NO:
31):
atgaaattagctgtttacggaaaaggtggtattggaaaatcaacgacaagttgtaatatttcgattgctttacga
aaacgtggtaaaaaagtgttacaaattggttgtgatcctaaacatgatagtacttttacattgacagggttttta
attccaaccattattgatacattaagttctaaagattatcattatgaagatatttggcccgaagatgttatttac
ggaggttatgggggtgtagattgtgttgaagctggaggaccacctgccggtgcggggtgtggtggttatgttgta
ggtgaaacggtaaaacttttaaaagagttaaatgcttttttcgaatacgatgttattttatttgatgttttaggt
gatgttgtttgtggtggctttgctgctccattaaactacgctgattattgtattattgtaactgataatggtttt
gatgctttatttgctgcaaatcgtattgcagcttcagttcgtgaaaaagcacgtacacatccattgcgtttagcg
ggtttaatcggaaatcgtacatcaaaacgtgatttaattgataaatatgtagaagcttgtcctatgccagtatta
gaagttttaccattaattgaagaaattcgtatttcacgtgttaaaggcaaaactttatttgaaatgtcaaataaa
aataatatgacttcggctcatatggatggctctaaaggtgacaattctacagtaggagtgtcagaaactccatcg
gaagattatatttgtaatttttatttaaatattgctgatcaattattaacagaaccagaaggagttattccacgt
gaattagcagataaagaactttttactcttttatcagatttctatcttaaaatttaa
Photochlorophyllide reductase subunit L (chIL) amino acid sequence (SEQ ID NO: 32):
MKLAVYGKGGIGKSTTSCNISIALRKRGKKVLQIGCDPKHDSTFTLTGFLIPTIIDTLSSKDYHYEDIWPEDVIY
GGYGGVDCVEAGGPPAGAGCGGYVVGETVKLLKELNAFFEYDVILFDVLGDVVCGGFAAPLNYADYCIIVTDNGF
DALFAANRIAASVREKARTHPLRLAGLIGNRTSKRDLIDKYVEACPMPVLEVLPLIEEIRISRVKGKTLFEMSNK
NNMTSAHMDGSKGDNSTVGVSETPSEDYICNFYLNIADQLLTEPEGVIPRELADKELFTLLSDFYLKI
Photochlorophyllide reductase subunit N (ch1N) nucleic acid sequence (SEQ ID NO:
33):
atgttagatggtgccacaacgattttaaatttaaatagtttttttgaatgtgaaactggcaattatcatactttt
tgcccgattagctgtgtagcttggttatatcaaaaaatcgaagatagcttttttttagtaattgggacaaaaaca
tgtggttattttttacaaaatgcccttggagttatgatttttgccgaacctaggtatgctatggcagaattagaa
gaaagtgatatttcagcacaattaaacgattataaagaattaaaacgtttatgtttacaaattaaacaagataga
aatcccagcgttattgtttggattggaacttgtacaactgaaattatcaaaatggatttagaagggatggctcca
cgtttagaaactgaaatcggcatacccattgttgtagcacgtgctaatggtttagattatgcttttacacaaggt
gaagacacagttttatcagcaatggccttagcatccttaaaaaaagatgttccttttttagtaggtaatactggg
ttaacaaacaaccagcttctccttgaaaaatcaacttcttcagttaatgggacagacggaaaggaattacttaaa
aaatctcttgtattatttggttccgtaccaagtacagttactacacaattaactttagaattaaaaaaagaaggt
attaatgtatctggatggcttccatctgctaattataaagatttacctacttttaataaagatacacttgtatgt
ggtataaatccttttttaagtcgaacagctaccacgttaatgcgtcgtagtaagtgcacattaatttgtgcaccc
tttccaataggccccgatggcacaagagtttggattgaaaaaatttgtggtgcttttggcattaatcctagtctt
aatccaattactggtaatactaatttatatgatcgtgaacaaaaaattttcaacgggctagaagattatttaaaa
ttattacgtggaaaatctgtattttttatgggtgataatttattagaaatttctttagcacgttttttaacacgt
tgtggtatgattgtttatgaaatcggaattccatatttagataaacgatttcaagcagcagaattagctttatta
gaacaaacttgtaaagaaatgaatgtaccaatgccgcgcattgtagaaaaaccagataattattatcaaattcga
cgtatacgtgaattaaaacctgatttaacgattactggaatggcacatgcaaatccattagaagctcgaggtatt
acaacaaaatggtcagttgaatttacttttgctcaaattcatggatttactaatacacgtgaaattttagaatta
gtaacacagcctcttagacgcaatctaatgtcaaatcaatctgtaaatgctatttcttaa
Photochlorophyllide reductase subunit N (ch1N) amino acid sequence (SEQ ID NO: 34):
MLDGATTILNLNSFFECETGNYHTFCPISCVAWLYQKIEDSFFLVIGTKTCGYFLQNALGVMIFAEPRYAMAELE
ESDISAQLNDYKELKRLCLQIKQDRNPSVIVWIGTCTTEIIKMDLEGMAPRLETEIGIPIVVARANGLDYAFTQG
EDTVLSAMALASLKKDVPFLVGNTGLTNNQLLLEKSTSSVNGTDGKELLKKSLVLFGSVPSTVTTQLTLELKKEG
INVSGWLPSANYKDLPTFNKDTLVCGINPFLSRTATTLMRRSKCTLICAPEPIGPDGTRVWIEKICGAFGINPSL
NPITGNTNLYDREQKIFNGLEDYLKLLRGKSVFFMGDNLLEISLARFLTRCGMIVYEIGIPYLDKRFQAAELALL
EQTCKEMNVPMPRIVEKPDNYYQIRRIRELKPDLTITGMAHANPLEARGITTKWSVEFTFAQIHGETNTREILEL
VTQPLRRNLMSNQSVNAIS
Porphobilinogen deaminase (PBGD1) nucleic acid sequence (SEQ ID NO: 35):
atgcagcagtgcgttggccgctccgtccgcgctccgtccagcagggcggtcgcgcccaaggtcgctggcgctcgt
gtcagccgccgcgtgtgccgcgtctatgcctccgctgttgctaccaagacggtgaagattggcacgcgcggctcg
cccctggctctggcccaggcttacatgactcgcgacctgctgaagaagagcttccctgagctgagcgaggagggt
gctctggagatcgtgatcatcaagaccaccggtgacaaaatcctgaaccagcccctggctgacatcggtggcaag
ggtctgtttaccaaggagatcgatgatgctctgctgagcggcaagattgacatcgccgtgcactccatgaaggac
gtgcccacctacctgcccgagggcaccatcctgccctgcaacctgccccgcgaggatgtgcgcgatgtgttcatc
tcgcctgtcgccaaggacctgagcgagctgcccgccggcgccattgtgggctcggcctcgctgcgccgtcaggcc
cagatcctggccaagtacccccacctcaaggtggagaacttccgcggcaacgtgcagacccgcctgcgcaagctg
aacgagggcgcctgctccgccaccctgctggctctggccggtctgaagcgcctggacatgactgagcacatcacc
aagaccctcagcattgacgagatgctgcccgccgtgagccagggcgccattggcattgcctgccgcaccgacgac
ggcgccagccgcaacctgctggccgccctgaaccacgaggagacccgcatcgccgtggtgtgcgagcgcgccttc
ctgaccgccctggacggctcttgccgcacccccattgccggctacgcgcacaagggcgccgacggcatgctgcac
ttcagcggcctggtggccaccccggacggcaagcagatcatgcgcgctagccgcgtggtgcccttcacggaggcg
gatgccgtcaagtgcggcgaggaggccggcaaggagctcaaggccaacggccccaaggagctgttcatgtactaa
Porphobilinogen deaminase (PBGD1) amino acid sequence (SEQ ID NO: 36):
MQQCVGRSVRAPSSRAVAPKVAGARVSRRVCRVYASAVATKTVKIGTRGSPLALAQAYMTRDLLKKSFPELSEEG
ALEIVIIKTTGDKILNQPLADIGGKGLFTKEIDDALLSGKIDIAVHSMKDVPTYLPEGTILPCNLPREDVRDVFI
SPVAKDLSELPAGAIVGSASLRRQAQILAKYPHLKVENFRGNVQTRLRKLNEGACSATLLALAGLKRLDMTEHIT
KTLSIDEMLPAVSQGAIGIACRTDDGASRNLLAALNHEETRIAVVCERAFLTALDGSCRTPIAGYAHKGADGMLH
FSGLVATPDGKQIMRASRVVPFTEADAVKCGEEAGKELKANGPKELFMY
Porphobilinogen deaminase (PBGD2) nucleic acid sequence (SEQ ID NO: 37):
atgcgatcgtatctgctcaaggctcaagtggcctcatgtcagttttcgcgcacgtcgaaggtctggagactggcg
ccgggttctgacagacgacggtgtcggggcctcactcggacaccgcactgcgcggcccccaccagcgagcccgcc
ccgccatccagcagcggcaagagcgggcaacgaccactcgtgatagccacgcggccatctaagcttgcaaaggag
cagacgcggcaggtgcagcagctgctgctggcggcggcgcagctcaaggacgagcagctgcagctgagcaccctg
gaactggcgtctaggggcgacacgactcagggtgtgtcgctgcgcagtctgggctcgggcgcattcaccgaggag
ctggaccaggctgtgctgtcgggcgctgccgacatgtcggtgcacagcctgaaggactgccccgccgccctggcg
cccgggctgctgctggccgcctgcctgccgcgggccgacccccgggacgtcctcatcgcgcccgaggccacctcg
ctgggcgagctggtgccgggcagccgtgtgggcaccagcagcagccgccgcgcggcgcagatcaagcactccttc
ccccacctgcaggttgtgcagctgcgcggcaatgtggactcgcggctggggcgcatccgcagccgcgacatcggc
gccacagtgctggcggcggcgggcctcaagcggctgggtgtgatgaactcggacgagggtgacactaccgctacg
ggcgccgtgggggtggtgtgcagggcagacgatgagtgggtggtcggcctgctggacgccatctcgcaccgcggc
acggccctggaggtggcggcggagcgggcgtgcctggcagcgctgctgggcggcggcggcgcgtgccagcgttca
gcgttcccggacattgcgtgggcctgccacacgcggcacgaccccgacagcaacacaatggacctggattgcctg
gtggcggacctggagggcaaggagctcttcaggtacacggagttctaccggccggtcattgacgaggtggacgcg
gtgtcgctggggtcgctgtacggcagcctgctgcgcatgatggcgccaccaggcgcggccccctgttggcagcta
ccttcctcgcggcattag
Porphobilinogen deaminase (PBGD2) amino acid sequence (SEQ ID NO: 38):
MRSYLLKAQVASCQFSRTSKVWRLAPGSDRRRCRGLTRTPHCAAPTSEPAPPSSSGKSGQRPLVIATRPSKLAKE
QTRQVQQLLLAAAQLKDEQLQLSTLELASRGDTTQGVSLRSLGSGAFTEELDQAVLSGAADMSVHSLKDCPAALA
PGLLLAACLPRADPRDVLIAPEATSLGELVPGSRVGTSSSRRAAQIKHSFPHLQVVQLRGNVDSRLGRIRSRDIG
ATVLAAAGLKRLGVMNSDEGDTTATGAVGVVCRADDEWVVGLLDAISHRGTALEVAAERACLAALLGGGGACQRS
AFPDIAWACHTRHDPDSNTMDLDCLVADLEGKELFRYTEFYRPVIDEVDAVSLGSLYGSLLRMMAPPGAAPCWQL
PSSRH
Protoporphyrinogen oxidase (PPX1) nucleic acid sequence (SEQ ID NO: 39):
atgatgttgacccagactcctgggaccgccacggcttctagccggcggtcgcagatccgctcggctgcgcacgtc
tccgccaaggtcgcgcctcggcccacgccattctcggtcgcgagccccgcgaccgctgcgagccccgcgaccgcg
gcggcccgccgcacactccaccgcactgctgcggcggccactggtgctcccacggcgtccggagccggcgtcgcc
aagacgctcgacaatgtgtatgacgtgatcgtggtcggtggaggtctctcgggcctggtgaccggccaggccctg
gcggctcagcacaaaattcagaacttccttgttacggaggctcgcgagcgcgtcggcggcaacattacgtccatg
tcgggcgatggctacgtgtgggaggagggcccgaacagcttccagcccaacgatagcatgctgcagattgcggtg
gactctggctgcgagaaggaccttgtgttcggtgaccccacggctccccgcttcgtgtggtgggagggcaagctg
cgccccgtgccctcgggcctggacgccttcaccttcgacctcatgtccatccccggcaagatccgcgccgggctg
ggcgccatcggcctcatcaacggagccatgccctccttcgaggagagtgtggagcagttcatccgccgcaacctg
ggcgatgaggtgttcttccgcctgatcgagcccttctgctccggcgtgtacgcgggcgacccctccaagctgtcc
atgaaggcggccttcaacaggatctggattctggagaagaacggcggcagcctggtgggaggtgccatcaagctg
ttccaggaacgccagtccaacccggccccgccgcgggacccgcgcctgccgcccaagcccaagggccagacggtg
ggctcgttccgcaagggcctgaagatgctgccggacgccattgagcgcaacatccccgacaagatccgcgtgaac
tggaagctggtgtctctgggccgcgaggcggacgggcggtacgggctggtgtacgacacgcccgagggccgtgtc
aaggtgtttgcccgcgccgtggctctgaccgcgcccagctacgtggtggcggacctggtcaaggagcaggcgccc
gccgccgccgaggccctgggctccttcgactacccgccggtgggcgccgtgacgctgtcgtacccgctgagcgcc
gtgcgggaggagcgcaaggcctcggacgggtccgtgccgggcttcggtcagctgcacccgcgcacgcagggcatc
accactctgggcaccatctacagctccagcctgttccccggccgcgcgcccgagggccacatgctgctgctcaac
tacatcggcggcaccaccaaccgcggcatcgtcaaccagaccaccgagcagctggtggagcaggtggacaaggac
ctgcgcaacatggtcatcaagcccgacgcgcccaagccccgtgtggtgggcgtgcgcgtgtggccgcgcgccatc
ccgcagttcaacctgggccacctggagcagctggacaaggcgcgcaaggcgctggacgcggcggggctgcagggc
gtgcacctggggggcaactacgtcagcggtgtggccctgggcaaggtggtggagcacggctacgagtccgcagcc
aacctggccaagagcgtgtccaaggccgcagtcaaggcctaa
Protoporphyrinogen oxidase (PPX1) amino acid sequence (SEQ ID NO: 40):
MMLTQTPGTATASSRRSQIRSAAHVSAKVAPRPTPFSVASPATAASPATAAARRTLHRTAAAATGAPTASGAGVA
KTLDNVYDVIVVGGGLSGLVTGQALAAQHKIQNFLVTEARERVGGNITSMSGDGYVWEEGPNSFQPNDSMLQIAV
DSGCEKDLVEGDPTAPREVWWEGKLRPVPSGLDAFTEDLMSIPGKIRAGLGAIGLINGAMPSFEESVEQFIRRNL
GDEVFFRLIEPFCSGVYAGDPSKLSMKAAFNRIWILEKNGGSLVGGAIKLFQERQSNPAPPRDPRLPPKPKGQTV
GSFRKGLKMLPDAIERNIPDKIRVNWKLVSLGREADGRYGLVYDTPEGRVKVFARAVALTAPSYVVADLVKEQAP
AAAEALGSFDYPPVGAVTLSYPLSAVREERKASDGSVPGFGQLHPRTQGITTLGTIYSSSLFPGRAPEGHMLLLN
YIGGTTNRGIVNQTTEQLVEQVDKDLRNMVIKPDAPKPRVVGVRVWPRAIPQFNLGHLEQLDKARKALDAAGLQG
VHLGGNYVSGVALGKVVEHGYESAANLAKSVSKAAVKA
Uroporphyrinogen III decarboxylase (UROD1) nucleic acid sequence (SEQ ID NO: 41):
atgcagaccaaggctttcacctctgcgcgcccccagcgggccgctgcgctcaaggcgcagcgcacctcgtcggtg
accgtgcgcgcgaccgcggcccccgccgtggcctctgcccccgccgcctcgggctctgcctctgaccccctgatg
ctgcgcgccatccgcggcgacaaggtggagcgcccgcccgtgtggatgatgcgccaggccggccgctaccagaag
gtgtaccaggacctgtgcaagaagcaccccacgttccgtgagcgctcggagcgcgtggacctggcggtggagatc
tctctgcagccgtggcacgcgttcaagcccgacggcgtcatcctgttcagcgacattctgacccccctgcccggc
atgaacatccccttcgacatggcgcccggccccatcatcatggaccccatccgcaccatggcgcaagtggagaag
gtgacgaagctggacgctgaggccgcctgccccttcgtgggcgagtcgctgcgccagctgcgcacctacatcggc
aaccaggccgcggtcctgggcttcgtgggcgcccccttcaccctggccacctacattgtggagggcggcagctcc
aagaacttcgcgcacatcaagaagatggctttctccacccccgagatcctgcacgccctgctggacaagctggct
gacaacgtggccgactacgtccgctaccaggccgacgccggcgcccaggtggtgcagatcttcgactcgtgggcc
agcgagctgcagccccaggacttcgacgtgttctccggcccctacatcaagaaggtgatcgacagcgtgcgcaag
acccaccccgacctgcccatcatcctctacatcagcggctctggcggcctgctggagcgcatggcctcttgctcg
cccgacatcatctcgctggaccagtcggtggacttcaccgacggcgtcaagcgctgcggcaccaacttcgccttc
cagggcaacatggaccccggcgtcctgttcggctccaaggacttcatcgagaagcgcgtcatggacaccatcaag
gctgcccgcgacgccgacgtgcgccacgtgatgaacctgggccacggcgtgctgcccggcacccccgaggaccac
gtgggccactacttccacgtcgcccgcaccgcccacgagcgcatgtaa
Uroporphyrinogen III decarboxylase (UROD1) amino acid sequence (SEQ ID NO: 42):
MQTKAFTSARPQRAAALKAQRTSSVTVRATAAPAVASAPAASGSASDPLMLRAIRGDKVERPPVWMMRQAGRYQK
VYQDLCKKHPTFRERSERVDLAVEISLQPWHAFKPDGVILFSDILTPLPGMNIPFDMAPGPIIMDPIRTMAQVEK
VTKLDAEAACPFVGESLRQLRTYIGNQAAVLGFVGAPFTLATYIVEGGSSKNFAHIKKMAFSTPEILHALLDKLA
DNVADYVRYQADAGAQVVQIFDSWASELQPQDFDVFSGPYIKKVIDSVRKTHPDLPIILYISGSGGLLERMASCS
PDIISLDQSVDFTDGVKRCGTNFAFQGNMDPGVLFGSKDFIEKRVMDTIKAARDADVRHVMNLGHGVLPGTPEDH
VGHYFHVARTAHERM
Uroporphyrinogen III synthase (HEM4) nucleic acid sequence (SEQ ID NO: 43):
atgtcggccctggacgccgccgccatcccctacgagctagtgccgggtgtgtcctccgctctggccgccccgctg
ttcgccggcgtcccgctcacacacgtcagcctgagcccctcgttcaccgtggtcagcgggcacgacgtggccggc
accgactgggcggcgttccgggggctgcccacgctggtggttctgatggcgggtcgtaacctggggcagatagcc
cggcggcttgtgcaggacgcggggtgggcgcccgatacacctgtaagtcaacctagtggctag
Uroporphyrinogen III synthase (HEM4) amino acid sequence (SEQ ID NO: 44):
MSALDAAAIPYELVPGVSSALAAPLFAGVPLTHVSLSPSFTVVSGHDVAGTDWAAFRGLPTLVVLMAGRNLGQIA
RRLVQDAGWAPDTPVSQPSG
CHLD 5′ untranslated region (regulatory region) (SEQ ID NO: 45):
ggcgtccccacaaccaggacagcctacttcttgaccttattaataagtcgctgcgtgtcgcgactgaccattttg
gcccggacttgcgtgcttgtgatttgtgcttcgactagatccgcgggcaccaagggacgcggacagctgatagtc
aagaactagatcctctgggagcgtctggggctgtccccgctgctcgccaaggaa
CHLD 3′ untranslated region (regulatory region) (SEQ ID NO: 46):
gtgccgagtgactgaggtggcaaggtgcagtggcggcggaggcagttgtgctggggtggcaaggcggacaggcga
agctggtgggttgcgacgaggaggaggtgcacgtgcacgcgtaacataagaagaacagtgggaggacaggtagcg
tgacttgactgggacgaggagcgtactgatgtgtggcgtgtgttggtatgtgagcgttacccctcccctagatag
cggcggtctccactttcaggaggatgagagccatcatgaggctttgagggggcactggttcgtgtgtaggctgag
gctgctgttgaagtcacaaggcagcactgcatgcgcgagtgagtgtggccggatatgcatcgagttgcaggtaca
ctgaaatgaggtgactgcggcgtatatcgctgccagtacaggttgaagcggcgggcacggtgaatggagtactcg
gcctggaacgcttgcgatcagatggtcgagctcaagaagatttggttgagccgttgggtcgtgcgtcatattatg
gcttgcatcttcggggagcggcaagaaacggactccaatgcaggccctcgggcgagaaagattgggcgtgtccgg
gggtgcattctcgccgcgtggggctgcatcgaatttcgcttgagtgccccttcccggggagggggggcggtagtt
caaccccatcatcgtaggggggttgtaaatgccagcccaaactaaa
CHLD Exon 1 (SEQ ID NO: 47):
atgaagtctctctgccatgagctcgctggccccagcgttactgggtgcggccggcgaagcctccggaaggctttc
agcggtgccaagattgcgcaggtctctcgccccgctgtgcttaacagcgtgcagcgccaacagcgtctcgcctgt
tctgccgtggccgagctctccgctgctgagctgcgcg
CHLD Exon 2 (SEQ ID NO: 48):
ccatgaaggtgtctgaggaggactccaagggcttcgatgcggatgtgtcgacccgcctggcccgctcgtaccctc
tggcggccgtggtgggccaggacaacatcaagcaggcgctgctgctgggcgccgtggacaccgggctgggcggca
tcgccatcgccggtcgccgcggtaccgccaagtccatcatggctcgcggcctgcacgctctgctgccgcccattg
aggtggtggagggcagcatctgcaacgccgaccccgaggacccccgctcctgggag
CHLD Exon 3 (SEQ ID NO: 49):
gctggcctggctgagaagtatgcgggcggccctgtgaagaccaagatgcgctcggcgccgtttgtgcagatccct
ctgggtgtgactgaggaccgcttggtgggcactgtggacattgaggcgtccatgaag
CHLD exon 4 (SEQ ID NO: 50):
gagggcaagactgtgttccagcccggcctgctggctgaggcgcaccgcggcatcctgtacgtggacgagatcaac
ctgctggatgacggcattgccaacctgctgctgtccatcctgtcggacggagtcaacgtggtggagcgcgagggc
atctccatcagccaccc
CHLD exon 5 (SEQ ID NO: 51):
ctgccggccgctgctgattgccacctacaaccccgaggagggccctctgcgtgagcacctgctggaccgcatcgc
cattggcctcagcgccgacgtccccagcaccagcgacgagcgcgtcaaggc
cattgacgcagccatccgcttccaggacaagccgcag
CHLD exon 6 (SEQ ID NO: 52):
gacactattgacgacacc gcggagctcaccgacgccctgcgcacctcg
CHLD exon 7 (SEQ ID NO: 53):
gtcatcctggctcgcgagtacctgaaggacgtgaccatcgcgccggagcaggtgacctacattgtggaggaggcg
cgccgcggcggagtccaggggcacc gcgcggagctgtacgcggtcaag
CHLD exon 8 (SEQ ID NO: 54):
tgtgccaaggcgtgtgcggctctggagggccgtgagcgtgtgaacaaggatgacctgcgccaggccgtgcagctg
gtcatcctgccgcgcgccaccatcctggaccagcccccgcccgagcaggagcagcccccgccgccgcccccgccc
cctcccccgccgccgccgcag
CHLD exon 9 (SEQ ID NO: 55):
gaccaaatggaggacgaggaccaggaggagaaggaggacgagaaggaggaggaggagaaggagaacgaggaccag
gacgagcccgag
CHLD exon 10 (SEQ ID NO: 56):
atccctcaggagttcatgtttgagtccgagggcgtcatcatggacccctccatcctcatgttcgcgcagcagcag
cagcgcgcgcagggccgctccggccgcgccaagacgctcatcttcagcgacgaccgcggccgctacatcaagccc
atgctgcccaagggtgacaaggtcaagcgcctggcagtggacgccacgcttcgcgccgccgcgccctaccagaag
CHLD exon 11 (SEQ ID NO: 57):
attcgccggcagcaggccatcagcgagggcaaggtgcagcgcaaggtgtacgtggacaagccagaca
CHLD exon 12 (SEQ ID NO: 58):
tgcgctccaagaagctggcccgcaaggccggtgcgctggtgatttttgttgtggacgcgtccggctccatggctc
tgaaccgcatgagcgccgccaagggcgcctgcatgcgcctgctggctgagtcgtacaccagccgcgaccaggtgt
gcctcatccccttctacggcgacaaggccgaggtgctgctgccgccctccaagtccatcgccatggcccgccgcc
gcctggactcgctgccctgcggcggcggctcgccccttgcgcacggcctgtccacggcggtacgtgtgggcatgc
aggccagccaggcgggcgaggtgggccgcgtcatgatggtgctcatcacggacggccgcgccaacgtcagcctgg
ccaagtccaacgaggaccccgaggcgctcaagcccgacgcgcccaagcccaccgccgactcgctgaaggacgagg
tgcgcgacatggccaagaaggccgcgtccgccggcatcaacgtgcttgtcattgacacggagaacaagttcgtga
gcaccggctttgcggaggagatctccaaggcagcgcagggcaagtactactacctgcccaacgccagcgacgccg
ccatcgcggcggccgcgtccggcgccatggccgcggccaagggcggctactag
CHLD Intron 1 (SEQ ID NO: 59):
gtgagcgcctactttgatatgtaccaaagataccactgataggtttaggcacggaagatctggacttggaccccg
tttgcgcaagccgggcgatgcacccatttcgcggtcacgccgagcgctggggtgcaatttagcgtgcccgacaag
ctagaaaacagggaattaccatttgtttaattttgttgcgagagatctttgcttgtgtccaccggccgcgcgggg
gaacttccggtgttgcgcaaggttgcgtgcgtgcccaccatcaacacctgtgccaggtctgtgtcacccccaggt
tccaccaccctgcaatcttccaattgtgtctcgtttgctcgttgtctaatagtcgtcctttgctcatccctacct
gcag
CHLD Intron 2 (SEQ ID NO: 60):
gtgaggcagggaaggtgacacaggaggttttgaaagagagacagggaggcaaagatggatggcggggcgggcagt
gactttggggcggcatggagtgggattggtggagtgggattgggcaccatgtatcacagatgttggcaacacagc
gcagggccttgctctgtgcttgtgttgaccgtctagtcccccgtgccctgaaccaagtctttcctcctgacacgg
tcctccatgtcctccttccggcattcccttcctcgtccacag
CHLD Intron 3 (SEQ ID NO: 61):
gtgagccagcaagggaggagaggggaacggccgggtagggcagccggagtttaaccacgccaattcaacggggag
caacggggaagaggaagggccggaagaggacggcaaaagcatttggtgggggcagcggctgtagtcagaagcgca
aaggctgccacagtgtggcccgcaccctcctcaccaccagtttggcatgatcgtttagcatgggctggaatactc
accgccagttctctcctctcccctctcctcccctgtccccgcctgcag
CHLD Intron 4 (SEQ ID NO: 62):
gtgagtgcgcgcgctgggtgtgtttgtgggacggcgcggcattggagcgcaggtgcgggtgctgggccgtgcact
tgtccgttggttcccttggaagcttcgatacacactcttactgcacgctctttaaccgccccccccctccacctc
tgcccgccccgtgcag
CHLD Intron 5 (SEQ ID NO: 63):
gtgggtgggggaaagtgactggatgtcggtgggttttaggtatgtgcgtgtgtacgatgcggggagcagtacgga
agcgggcacgagcggtgagggggcaggattgtggcgcacgctcgggccaagcccgggctcgcgacagagggtggg
cttgtattcgtagtcaagcgcatcaggaagtgcagttgactggattcacctgaaacggcgctgagcgggcggcta
atagaatcccgcttcctgtccgcccctccccttgcccttcaatccgtcag
CHLD Intron 6 (SEQ ID NO: 64):
gtgagtggcgggggccgtgcgtttgtttgttgcgtgggctggctggctggctttgttggatgagggcgctgctca
ccactcatctctttgaatccccacttatccagttgcctgcatgaaaccccgcctgactcactccccaccatcctg
taccgcttttccaaacatccttgcaaccatcccgccatccccacccgcag
CHLD Intron 7 (SEQ ID NO: 65):
gtgaggagttggagggggaaggggcgaggggatgcgacagaagcgagggcgaggggagccggggtgggttgttgc
aagtgtcgtgaattatagaatgaccccaaaagcgccggcccaacagggcctattacttgcgagtcaatccaaccc
ctgatatagggagaatggggtagaggtcgtatcacgacagcaaggatgtacagtgggccttggggttgggaggta
cagggaaaaaggagaggacatggggttgggtaagcggggaataacaaatatacacccagcgtttatggaagtggg
agatggaaacgggggcggacgaacaggaacaggggccggatggaggggctatgggggcatggtgggtgggggtac
ggcgcggggcagagcagggtcttgggtgaatgggcaagatgctgatgcttgggatgaagacactatgagcaaaga
aatggttgttgacgattgccatgatcatcgcagtgggggaggcggggtggcaataccggcagtcaacagttgggg
tgcgatcaagattgattggagtaccagcagtggccgggatctggctgacgtgtctcgagcgagttgctggggtgg
caaggagatgcaggggcagacgacgttgtgcgaccacacttacacacatttccttccccttgcgtgtgtccgtgc
gccctgtgcctccag
CHLD Intron 8 (SEQ ID NO: 66):
gtacgtaaacgtatttgattgctcaggtggttagccttggtgtggctgctgtttgacttgtgcagctgtctttgt
gtacatgttccacaaccctgtactccccatattccgcccccattccag
CHLD Intron 9 (SEQ ID NO: 67):
gtgagaggcggcgcggcggcttgcgggcgaaggcggggggcggggcggaggcaatgcggccgcgcatggccagca
acggaagggctggctatcaacacggcgagcgcacgatattcatataagagtgccatcgtgcaatgctgaatactt
gcgccaaccggatctcgctgctccgcttccaccggactgctttctcatctctccccttcaccctgtgtgtatcca
cag
CHLD Intron 10 (SEQ ID NO: 68):
gtgagtgcccgaggtggtgggtggtgaattggggcacgagggtatgtgggcctaagggagctgaatggggcatgt
tttcttctgagcatcacggtcagagcttgacctgtcctccccgctgtacccccgtgcacggtccgacacag
CHLD Intron 11 (SEQ ID NO: 69):
gtgagtacagcgcatcccggcgcaatcattgggcctagttactgctgcaggactcgtgtgctcttaagggctggc
agctgtcagaagctctactcctcgcactgaccactgtgcctttctctccttcctctctccctccccgcacccctc
ctcccacttcctcaacag
CHLI2 5′-untranslated region (regulatory region) (SEQ ID NO: 70):
gcagacttccataaagctcttgtaacgctgtaccaactagtaagcggtacaattcgcctgagcccgagcaacgcg
acctttcttgctctgtggatctctgataatctaaccagaccaaaaccttttcactaatctaggcaaca
CHLI2 3′-untranslated region (regulatory region) (SEQ ID NO: 71):
aaaaggctggtgtaggcctgtcgggtcgtgttaaaggttgctgcgtgaacgtgtaagtgtgacagtgtgccggta
tgtgtgtgtatacatgtgttgcggtgtgcttttgtggcggtacatggtgatgactgagcgggtgggacagagcac
ggttaactgacgagggcagtccgtgcgagacggacgtttttgtagccgaggtgcaaggactgatgacgggctaag
ctgctggagacttggagttgagagtgcaggtggatcgacggtttctctaaggagtatgaataggcaggagggctg
gagacatttggggtgcaaggaggcggtagtatgggagatgtccatgggcggattttggcctctgtaacttcttaa
cgccca
CHLI2 Exon 1 (SEQ ID NO: 72):
atgcagagtctccagggtcagcgcgcgttcactgcggtgcgccagggtcgggcgggtcccctgcggactcgcctg
gtcgtgcgctcgtctgttgccttgccatccacgaaagccgcgaagaagccgaacttcccgttcgtcaagattcag
ggccaggaggagatgaagcttgcactgctgctgaacgtggtcgaccccaacatcggcggagtgcttattatgggt
gaccgcggcactgccaagtcggtcgcg
CHLI2 Exon 2 (SEQ ID NO: 73):
gtccgcgccctggtggatatgcttcccgacattgacgtggttgagggcgacgccttcaacagctcccccaccgac
cccaagttcatgggccccgacaccctgcagcgcttccgcaacggcgagaagctgcccaccgtccgcatgcggacc
cccctg
CHLI2 Exon 3 (SEQ ID NO: 74):
gtggagctgcctctgggcgccaccgaggaccgcatctgcggcaccatcgacatcgagaaggcgctgacgcagggc
atcaaggcctacgagcccggcctgctg
CHLI2 Exon 4 (SEQ ID NO: 75):
gccaaggccaaccgcggcatcctgtatgtggacgaggtgaacctgctggatgatggcctg
CHLI2 Exon 5 (SEQ ID NO: 76):
gttgatgtcgtgctggactcgtcggctagcggcctgaacactgtggagcgtgagggtgtgtccattgtgcaccct
gcccgcttcatcatgattggctcaggcaacccccag
CHLI2 Exon 6 (SEQ ID NO: 77):
gagggtgagctgcgcccgcagctgctggatcgcttcggcatgagcgtcaacgtggccacgctgcaggacaccaag
cagcgcacgcagctggtgctggaccg
CHLI2 Exon 7 (SEQ ID NO: 78):
gcttgcgtacgaggcggaccctgacgcatttgtggactcgtgcaaggccgagcagacggcgctcacggacaagct
ggaggcggcccgccagcgcctgcggtccgtcaagatcagcgaggagctgcag
CHLI2 Exon 8 (SEQ ID NO: 79):
atcctgatctcggacatttgctcgcgcctggatgtggatggcctgcgcggtgacattgtgatcaaccgcgccgcc
aaggcgcttgtggccttcgagggccgcaccgaggtgaccacgaatgacgtggagcgcgtcatctcgggctgcctc
aaccaccg
CHLI2 Exon 9 (SEQ ID NO: 80):
cctgcgcaaggacccgctggaccccattgacaacggcaccaaggtggccatcctgttcaagcgcatgaccgaccc
cgagatcatgaagcgcgaggaggaggccaagaagaagcgcgaggaggcggccgccaaggccaaggcggagggcaa
ggcggaccgccccacgggcgccaaggctggcgcctgggctggcttgccccctcgtcggtaa
CHLI2 Intron 1 (SEQ ID NO: 81):
gtaggtaacacaagcaattatggggcgaagatctaggctccgctgatccgggcgggcaatcggcatcgtcggtgc
aaccgtggggcgtctgtgcaccctttgctggtgccaggttgcctgactcgcctgcattcctgtaccgagccacat
tggctgctttgcagcgtgcatgggacgggtgtaggataagcgctatgtatgcgatagcgcgggtgcaccggcttg
gcatggcaaggttgcggggtgcacatgcgtgccagcgtcccctcagcatcagagtctggatctaagggctcagcg
gcttcctgcgcatgtgggtctttgcgtagtgctacgaagccttataattaaagctcatgtattgagtggtccggg
tttggggcactagtagtgccaggaggcgcgtgccaggttgatatgagcatatcagcacccgttccttgcgaaacg
cttccgttgtgctcccttccccaccacctccccgctcatacccatacatatggctatccgtcctctcattgcttg
cccctacag
CHLI2 Intron 2 (SEQ ID NO: 82):
gtgagcgggcctaccttctgaagacagtcttacgtgttgcactgcagcggtgttgcgcacctctgcttttgcgtg
cgccgggaagcgcggattgcggcctcacagatcaagcccggaaacgcttgttgtttccagcgggtggcacacacg
cgcgcgcgcgcacagtgacaccctcacggccgcgctgccctgcag
CHLI2 Intron 3 (SEQ ID NO: 83):
gtgcgtagtgcatggggagaggggacgaggggaggagggcagggccaataaaccgaaccccaagtcatcgagaca
cagaacccgataatagctcccagatcgccaaggggtgaggcgggaagccaaggatgatgcgttggccgcattgcg
tgttgacgtcaggcttacacagggtctgactggctgtgcttggggtttggcacgcttcttgactggccccgtacg
catgctgcag
CHLI2 Intron 4 (SEQ ID NO: 84):
gtgagtggtggtggtttctgggtcagcagaggacttctgtagtaggtaatgtgggccagggaagtgtggctaaca
tgccaaacacgggggcgcaccagtgcaagctgcattcgctgacgtgcacgggtgcaatgggtgcaaggcgaactg
caatcgcggtgcacagttgccagggctgcgctcacgcttgagtgtctgcacacgcactgcag
CHLI2 Intron 5 (SEQ ID NO: 85):
gtgcgtagcgtgcgcgcatgtacttgtctcccttgtcatgttgggaaaggtcggtccccagcctgcttgcaagat
gcggccggtcagcagctgcggacggtcagcacctacgtgccgaggttgtgtaacatgaatggcgttggggcggcc
gacctgccacaagctgaactgcgaccagcaaggcagctgccagcaacgcacacccgacgtgctacacgcttgtgt
tttgacctcctaaacacacccgcccgctgtctgtcacgtccacag
CHLI2 Intron 6 (SEQ ID NO: 86):
gtaagcggcggcggcgcggggacacggagggacatttcgcgagcatgggttgaggagtcgggaggattcggtggc
tggccggagtcgggagtcggagtcgcgagtcggaagtcaagcttctggcggcttcgtgctgtcgggtgcgctcgc
catgatggcgctgaccggagggcgtcacgctgtgtatgtgggcgcgcag
CHLI2 Intron 7 (SEQ ID NO: 87):
gtacggggcgtacagcgggggcggctgcacggggccagtgaccgacagggcagcacgcggctggcgaagagcgac
aaagtgacagggtgaccaagaccgggtgatgccacgagaggggcgcgggagccgtgcattgggtcgaggagggag
gaatgcaactttacactgatgcctctgtatacggccgccttccgagccctgcaaaccttcgctttcccccgacgc
acgcag
CHLI2 Intron 8 (SEQ ID NO: 88):
gtgagcgcagcgtgcggtggatgcggtgcgcgtgcgggttgccaacttattattttgtacgtggacgcgtggctg
gcgatggcatgtcatggcgcgaatggatattgggcgaatggataccggtaatggtagcacggggcggcagggcct
ggcggtagtggggttgagggggcgaggactccagcgcgcgatacatgccatgttcagcatggccccaactgacag
cgcccgctgccctgtgcgccccgctccctccgcgcacccgctcctcctacacag
CHLH1 5′-untranslated region (regulatory region) (SEQ ID NO: 89):
ctagtctagagggaactagggaggggcaacagagaa
CHLH1 3′-untranslated region (regulatory region) (SEQ ID NO: 90):
gcggcctccccttcatggtagcactagttggcgggttgtggttggactaggcggctagggtatatacctagtagc
ggcggctgcggagtggagggctggcgcccagcgcgagggcgtggcctttcctcctggacccgagagcgctccgcg
aggagacggcgagtgagataggcagcagcgagcggagatcgatttgtgaacagttttgtggcgggatcccatagc
ggatgcagagaagaccttagagcagcttcctcggtggagtgaacgagccagagcggagggaaggcgcatgaggga
actgcagggactggaactgcgggagtgcaggtccggtgctaggtccgctaaacagtgcggtctacgcctgtgtgt
gaggtgtgcgtgtgtgtgtgagctgtgcggttttgttgtgcaaagtaggagtgagccgagccgcgcgtactttgt
ggcgtgtttggctgctggcgctgagagccaagagagggtaaacgggtttggtattttatggtgcggggtgaaagc
agccctcgcaggaatggagcgattctgcagcatgatgcacgtgtgcctgcgcgtggatggtggctgttgatatgg
ctctgccactccggcagcaccgctacgatacctagcggtgcctggagtggtctctctgtttggtgcgtgatgttt
gggtttgccgttttgattctttgtttcgtgctgaatggctgaggcggcaagacccctcgtgccagtgtacagagc
ctcacggctccctcggaccccgcgtggggacgtccattcccggtggcggtgtcgcctcggcggtgtaaagcaaaa
aatatttt
CHLH1 Exon 1 (SEQ ID NO: 91):
atgcagacttcctcgcttcttggccggcgcacggcccacccggctgcgggcgcgacgcccaagccg
CHLH1 Exon 2 (SEQ ID NO: 92):
gttgcgccctcgccccgcgtggctagcacccgccag
CHLH1 Exon 3 (SEQ ID NO: 93):
gtcgcgtgcaatgtggcgactggaccccggccgcccatgaccaccttcaccggtggcaacaagggccctgctaag
cagcaggtgtcgctggatctgcgcgacgagg
CHLH1 Exon 4 (SEQ ID NO: 94):
gcgctggcatgttcaccagcaccagcccggagatgcgccgtgtcgtccctgacgatgtgaagggtcgcgttaagg
tgaaggttgtgtacgtggtgctggaggcccagtaccagtcggccatcagcgctgcggtgaagaacatcaacgcca
agaactccaag
CHLH1 Exon 5 (SEQ ID NO: 95):
gtgtgcttcgaggtggtgggctacctgctggaggagctgcgtgaccagaagaacctcgatatgctcaaggaggat
gtggcctctgccaacatcttcatcggctcgctcatcttcattgaggagcttgccgagaag
CHLH1 Exon 6 (SEQ ID NO: 96):
attgtggaggcggtgagccccctgcgcgagaagctggacgcgtgcctgatcttcccgtccatgccggcggtcatg
aagctgaacaagctgggcacgttttcgatggctcagctgggccagtcgaagtcggtgttctcggagttcatcaag
tctgctcgcaag
CHLH1 Exon 7 (SEQ ID NO: 97):
aacaacgacaacttcgaggagggcttgctgaagctggtgcgcaccctgcctaaggtgctgaagtatctgccctcg
gacaaggcgcaggacgccaagaacttcgtgaacagcctgcagtactggctgggcggtaactcggacaacctggag
aacctgctgctgaacaccgtcagcaactacgtgcccgctctgaagggcgtggacttcagcgtggctgagcccacc
gcctaccccgatgtgggtatctggcaccctctggcctcgggcatgtacgaggacctgaaggagtacctgaactg
CHLH1 Exon 8 (SEQ ID NO: 98):
gtacgacacccgcaaggacatggtcttcgccaaggacgcccccgtcattggcctggtgctgcagcgctcgcacct
ggtgactggcgatgagggccactacagcggcgtggtcgctgagctggagagccgcggtgctaaggtcatccccgt
ctttgccg
CHLH1 Exon 9 (SEQ ID NO: 99):
gtggcctggacttctccgcccccgtcaagaagttcttctacgaccccctgggctctggccgcacgttcgtggaca
ccgttgtgtcgctgaccggcttcgcgctggtgggcggccccgcgcgccaggacgcgccgaaggccattgaggcgc
tgaagaacctgaacgtgccctacctggtgtcgctgccgctggtgttccagaccactgaggagtggctggacagcg
agctgggcgtgcaccccgtccaggtggctctgcag
CHLH1 Exon 10 (SEQ ID NO: 100):
gttgccctgcccgagctggatggtgccatggagcccatcgtgttcgctggccgtgactcgaacaccggcaagtcg
cactcgctgcccgaccgcatcgcttcgctgtgcgctcgcgccgtgaactgggccaacctgcgcaagaagcgcaac
gccgagaagaagctggccgtcaccgtgttcagcttcccccctgacaagggcaacgtcggcactgccgcctacctg
aacgtgttcggctccatctaccgcgtgctgaagaacctgcagcgcgagggctacgacgtgggcgccctgccgccc
tcggaggaggatctgatccagtcggtgctgacccagaaggaggccaagttcaactcgaccgacctgcacatcgcc
tacaagatgaaggtggacgagtaccagaagctgtgcccttacgccgaggcgctggaggagaactggggcaagccc
cccggcaccctgaacaccaacggccaggagctgctggtgtacggccgccagtacggcaacgtcttcatcggcgtg
cagcccaccttcggctacgagggcgacccgatgcgcctgctgttctcgaagtcggccagcccccaccacggcttc
gccgcctactacaccttcctggagaagatcttcaaggccgacgccgtgctgcacttcggcacccacggctcgctg
gagttcatgcccggcaagcaggtcggcatgtcgggtgtgtgctaccccgactcgctgatcggcaccatccccaac
ctctactactacgccgccaacaacccgtctgaggccaccatcgccaagcgccgctcgtacgccaacaccatttcg
tacctgacgccgcctgccgagaacgccggcctgtacaagggcctgaaggagctgaaggagctgatcagctcgtac
cagggcatgcgtgagtctggccgcgccgagcagatctgcgccaccatcattgagaccgccaagctgtgcaacctg
gaccgcgacgtgaccctgcccgacgctgacgccaaggacctgaccatggacatgcgcgacagcgttgtgggccag
gtgtaccgcaagctgatggagattgagtcccgcctgctgccctgcggcctgcacgtggtgggctgcccgcccacc
gccgaggaggccgtggccaccctggtcaacatcgctgagctggaccgcccggacaacaacccccccatcaagggc
atgcccggcatcctggcccgcgccattggtcgcgacatcgagtcgatttacagcggcaacaacaagggcgtcctg
gctgacgttgaccagctgcagcgcatcaccgaggcctcccgcacctgcgtgcgcgagttcgtgaaggaccgcacc
ggcctgaacggccgcatcggcaccaactggatcaccaacctgctcaagttcaccggcttctacgtggacccctgg
gtgcgcggcctgcagaacggcgagttcgccagcgccaaccgcgaggagctgatcaccctgttcaactacctggag
ttctgcctgacccag
CHLH1 Exon 11 (SEQ ID NO: 101):
gtggtcaaggacaacgagctgggcgccctggtagaggcgctgaacggccagtacgtcgagcccggccccggcggt
gaccccatccgcaaccccaacgtgctgcccaccggcaagaacatccacgccctggaccctcagtcgattcccact
caggccgcgctgaagagcgcccgcctggtggtggaccgcctgctggaccgcgagcgcgacaacaacggcggcaag
taccccgagaccatcgcgctggtgctgtggggcactgacaacatcaagacctacggcgagtcgctggcccaggtc
atgatgatggtcggtgtcaagcccgtggccgacgccctgggccgcgtgaacaagctggaggtgatccctctggag
gagctgggccgcccccgcgtggacgtggttgtcaactgctcgggtgtgttccgcgacctgttcgtgaaccagatg
ctgctgctggaccgcgccatcaagctggcggccgagcaggacgagcccgatgagatgaacttcgtgcgcaagcac
gccaagcagcaggcggcggagctgggcctgcagagcctgcgcgacgcggccacccgtgtgttctccaacagctcg
ggctcctactcgtccaacgtcaacctggcggtggagaacagcagctggagcgacgagtcgcagctgcaggagatg
tacctgaagcgcaagtcgtacgccttcaactcggaccg
CHLH1 Exon 12 (SEQ ID NO: 102):
ccccggcgccggtggcgagatgcagcgcgacgtgttcgagacggccatgaagaccgtggacgtgaccttccagaa
cctggactcgtccgagatctcgctgaccgatgtgtcgcactacttcgactccgaccccaccaagctggtggcgtc
gctgcgcaacgacggccgcacccccaacgcctacatcgccgacaccaccaccgccaacgcgcaggtccgcactct
gggtgagaccgtgcgcctggacgcccgcaccaagctgctcaaccccaagtggtacgagggcatgcttgcctcggg
ctacgagggcgtgcgcgagatccagaagcgcatgaccaacaccatgggctggtcggccacctcgggcatggtgga
caactgggtgtacgacgaggccaactcgaccttcatcgaggatgcggccatggccgagcgcctgatgaacaccaa
ccccaacagcttccgcaagctggtggccaccttcctggaggccaacggccgcggctactgggacgccaagcccga
gcagctggagcgcctgcgccagctgtacatggacgtggaggacaagattgagggcgtcgaataa
CHLH1 Intron 1 (SEQ ID NO: 103):
gtaggtgtaattagaaggatcaaaacctagcggcctgatctgggactgacggcctcgcgcttcaatcactctgat
gcag
CHLH1 Intron 2 (SEQ ID NO: 104):
gtaggcacggcagaatgctcaatgaacatgcagctacatatgtttgggatcatggctgatctctgtgcgacgggt
ccgcgcag
CHLH1 Intron 3 (SEQ ID NO: 105):
gtgagcagcgcggaccgagcaagcgctggcgatgcagttggatttgttgttcttgggtcaggcgctcgctcgatg
gccagcgcgtgtatttaatgggataagggttgagacaaagcatctcttcgggtaaaaatcttagttttcgacagc
acgttgagaggcatgcaacttgctctttcgcag
CHLH1 Intron 4 (SEQ ID NO: 106):
gtgggtaaggagttgcattatcagtgtggcatggtgttgcgggcgtctggggcgctgcaacagcggcatcgtgcc
gaactgaccgtgccgggctacccgcgtgcag
CHLH1 Intron 5 (SEQ ID NO: 107):
gtgcgctagggttggggtctggagggtgtggattgcgcccaagtgccctgttgcgcttggcggtcgctgtcatga
tgtgagggtgacgtagtgcactcaattgcctgctacgtcaccacctttgatgggctggatctgaggcaggtcagc
tcggttccctgctgcatccagtgtccctgtcgccctgcacgtttgacgctgttcccccttccgcactgtctcgct
ttgcag
CHLH1 Intron 6 (SEQ ID NO: 108):
gtgtgggcacgcgctttgggaagggaggcatacatttttggttgcggttaggctgggcgcggacttggcactcac
acggtcattgcacactcatgtctcaccttcatttacggtcccttgtgccgaactacctacag
CHLH1 Intron 7 (SEQ ID NO: 109):
gtgagcagcatcagggcagagtgcatgaacggattggtggcagtggggaatggaattagacggacacgtctgggc
ggcaatatgttgcgctgcagtttttggggtgtagtgaactagaaaatagggaagagataggccacataacatccg
aaagctcatatttttgcaaccggcgcacctatcacagcccacctgaagggttttgtagtcaacgcgtgcaactga
ctagatgtccccttacctgtctgatttcag
CHLH1 Intron 8 (SEQ ID NO: 110):
gtgaggcggggcggcgctgccctcggtaggggttgcagatggtgatgggtaaccgaatgcatggccaatggggag
tgaaatcaggaaaggaggggtaacacaatgcagggcagcacctgaatcgtgaaggcggagttaggcagggatctg
tcagttcgcctgtcacgtggatgggcgcagctgacctttgtggtgttgtggtgtggcgcag
CHLH1 Intron 9 (SEQ ID NO: 111):
gtgagctcagctgggacatgtaggggctcgggtcgccggagcatcgatgtagaattacgggaggaggggagaggg
gagaggattgcacgaaccgagatgagggcggtggttcgggatttcgggcaaaagctcgtgcggcaagcgttcagt
gactgaagagcagtgtgcttcaactgcccctctgtccctcag
CHLH1 Intron 10 (SEQ ID NO: 112):
gtgcgaccggtgccgctgcgtggccaacagcttggtgccaccttcctgcggtgttgatttacactgtgtgcgtgg
atgtgttggtttttcgcaactttagtctgggctccagctctttgccttcattgatcactcgtcttacctcctgcg
ccatcatttgaatacag
CHLH1 Intron 11 (SEQ ID NO: 113):
gtgagccttaatgcaacacgtgtagccgttcgcatgggtggctgggtcatgctatggttggatcggcgtccgcct
gcttgctactgcctgttcggtaccagcgtttactgaccccgcgtgtgccattcccaccacctaccccctcgcctt
gcag
Ferrochelatase 5′-Untranslated region (regulatory region) (SEQ ID NO: 114):
gacagtgatatagcaataccgatataataggtttggcgggcttcaccttgtccttacccagaatgtggccctgac
agtcgatttccagcccccttgccactcgctccctgatttcttcaatcaactagttgggtcgttttctcgtaagg
Ferrochelatase 3′-Untranslated region (regulatory region) (SEQ ID NO: 115):
gggggcgggtggcgagtaaggcgtatggcggagcgaggagatgggctgtggcgtggccggtgttcttttgtgtga
ttggaaacatagacggggtgcggcacgcggcctgactgctgcgcggttggtgtggttgcggggggagcggggtcg
atggggcagcgcgcacgagttggttgaaggaggagggccaggcgctgggctacacccatggtttgaggatgctag
tgagtgatgtgtgcggggggcatggtgtgtaccattcagagtccagatgcacgcacggttgcgtgggagcgttcc
ctgctgtgcatgatgatggcgccttcgatgaatcatctcttgaaggtccaaatgaaacgtctgaagtctgcagag
ggtggtgctggacatgccatccaggcggaagtgggcagctgtgtctgactacaaagtaggtcttgttttgcttgg
atagcgtttggctatgtagcgtgtattctgctcatcaatcacgccaggcgtcagggactacccatgcaagtcggg
agcgtggctggctctggaaaagttgtagctgctaggtggcgttggctggggtgtcatgcatctcggcaggtaggc
ggtagcggtggacgacctctgcagcggagcatgtgcacaagatgtgactgcgcatgcacccgtatatgacggcgt
tggcgtcagttgttgagagtgaacagaggagagacgagcgaagctgccatgcccttagtggctggtgcgagaggg
gaagaaagagagaggaaggactttgcggcagtgccccacgccggagttggggacacggtcatcaacagggcggcg
gagctgggcggagtgggtgtgtgatgggacagggttcaaggcaggttggcgaggtcggagtgggtagaccagtcc
ttcagtgcaagggcattagggcatgatgtaagggctgaagcttg
Ferrochelatase Exon 1 (SEQ ID NO: 116):
atggcgtcgtttggattgatgcaaaggacggtgcactgtccccagcttgtggaggagcggtgttcgccggtcgct
ggctgctctggtcgtggcctgccagttatccagcggcaacg
Ferrochelatase Exon 2 (SEQ ID NO: 117):
gcgtggcgtgtgcagtgccaccaacggtgtccagcgagggcgtgtgctgcgccggacggccgcttcgaccgacgt
ggtctccttcgtggaccccaatgacattagaaaacccgcagcagcagcagctggccctgcggtggataaggtcgg
cgttctgctgttaaaccttggcgggcccgaaaagctcgacgacgtcaagcctttcctgtataacctattcgccga
cccagaaattattcgcctgccagcggcagctcagttcctgcagccgctgctcgcgacgatcatctccacgcttcg
cgccccgaagagcgcggagggctatgaggccattggcggtggtagcccgttgcgtaggattacagacgagcaggc
ggaggcgctggcggagtctctgcgcgccaagggccaacctgcgaacgtgtacgtgggcatgcgctattggcaccc
ctacacggaggaggcgctggagcacattaaggccgacggcgtcacgcgcctggtcatcctcccgctgtaccctca
gttctccatctctaccagcggctccagccttcgactgcttgagtcgctcttcaagagcgacatcgcgctcaagtc
gctgcggcacacggtcatcccgtcctggtaccagcggcggggctacgtgagcgcgatggcggacctgattgtaga
g
Ferrochelatase Exon 3 (SEQ ID NO: 118):
gagctgaagaagttccgggacgtgcccagcgtggagctgtttttctccgcgcacggcgtgcccaagtcctacgtg
gaggaggcgggcgacccatacaaggaggagatggaggagtgcgtgcggctcattacggacgag
Ferrochelatase Exon 4 (SEQ ID NO: 119):
gtcaagcggcgcggcttcgccaacacgcacacgctggcctaccagagccgcgtgggccccgcggaatggctcaag
ccgtacacggatgagtccatcaa
Ferrochelatase Exon 5 (SEQ ID NO: 120):
ggagctgggcaagcgcggcgtcaagtcgctgctggcggtgcccatcagctttgtcagcgagcacattgagacgtt
ggaggagatcgacatggagtaccgcgagctggcggaggagagcg
Ferrochelatase Exon 6 (SEQ ID NO: 121):
gcatccgcaactggggccgcgtgccggcgctgaacaccaacgccgccttcatcgacgacctggcggacgcggtga
tggaggcgctgccctacgtgggctgcctggccgggccgacagactcgctggtgccgctgg
Ferrochelatase Exon 7 (SEQ ID NO: 122):
gcgacctggagatgctgctgcaggcctacgaccgcgagcgccgcacgctgccgtcaccggtggtgatgtgggagt
ggggctggaccaagagcgcggagacgtggaacggccgcattgccatgattgccatcatcatcatcctggcgctgg
aggcagccagcggccagtccatcctcaaaaacctgttcctggcggagtag
Ferrochelatase Intron 1 (SEQ ID NO: 123):
gtgcgataataaatttgcatccttatgaattgctcaatgactaacgagcagcgtccgcgaccacag
Ferrochelatase Intron 2 (SEQ ID NO: 124):
gtgagggtggcattctgtaaagggagttgtggagttgggcagagcgagtgggtttggtcgccagggcgaggatgt
tgcgcgggcgttggcaggaacagggctgctagggcttgcgtggccagcgactagggtttcgactggccagcgccg
ccggggcgcgcttgccgaagctgcacagccccaagcgcttctgtggatcaaatggaaacttgtggcagtgtgtat
gctagcgccttggcgcaagaccaattttagtggtattactgttattactgtggtagcggtgggtattcggcggcg
tggttgttgttgcagccccgtgcgactaagaccgctggcaacgacagcaagccgccgcacccaggcatatacggc
ccaccagcaccaccgtacacaaccacgtgcctttgcactctacgcaccacagcgcgctgctgccgctcccacctc
ccatcccaacggcccctcttacccccacttcacaacccctcctctcacacgccctcctcttccccctcctcttcc
ag
Ferrochelatase Intron 3 (SEQ ID NO: 125):
gtgggccgggcgcagcgggcgggcgggaggggcaggaggggcaggaggggaggaagggaggggaggaagggatgg
aaagctggcgcagcggcagcggcgggacaggtagagggcgctgccccagcggcggcaggtgggcatggtgggcgg
gtaggggcgacgcgtgagggactcgtcaggcatccgcatggcggcgacttgctgctcctcaccgctgacggctgc
atctgctgtgtgcgtaacctggcctggctggcaccgcag
Ferrochelatase Intron 4 (SEQ ID NO: 126):
gtgaggcccgtgggtgggacgcggggagggacgcggggagggggagacgcgggagcgggacaagggtgaggatac
ggggagggaataggagaggccatggggagggatggggacacgggaggatgcacgggcctgggtggagccaggggg
aagtggacgacgagcccggcgggaggagggctgggtagaaggacgcgggaggtggttgggacaggtggacggggc
gtgtggagcatacggcgcaagaagcgggactgagcgggttgcagggatggatgtaatcacggcaagtaagaaccc
cgagtggggctcagcgtgtcagcctgccttatctttcgcgcaagcgctggggttttatttcgctgtacacacgtc
gcgcctttctgccgcag
Ferrochelatase Intron 5 (SEQ ID NO: 127):
gtgaggaggcgccggagttttgggggaaggggtgcggcgtgaagcgagatggcaggggcgaaggaaggagcggat
ggtggctgggtgcaagcggagaggcgacagagagtggaggttttggtggagcggttggggagaggggcgcagcag
ggatgcggccctggggatggcgggacagaagggagcaagtttgccaagtgaagggggggggtgctcaagaggaga
gggcggtggaggttaagacggccgtgctggttatgctggggttgcaaggcgcatgggcgcatggagccgggggag
tttggctgtggatgggcactgcggatgggcacggcttgctactcatgtgcggtcgcggtccgcggtgtgtcagcc
agccaggacccatcccactgggtcttcctgcgtgcctgggactgcttgccgccacccacccattcatcaccacca
ctgcgcagacccaccaacaccgctgccctgaactgctctgactcttggcgctcctcag
Ferrochelatase Intron 6 (SEQ ID NO: 128):
gtgagtcgcgccgtcgcggttggttcgcggatgccggttggcggatgacgttcggcggttggcattgggtttggg
tttgaggggttgttgggtgaggtcgggattggggtcgggattgggggtcgagcgtggggctggcgtggatgatgg
cgtggtctttggaaggggcttggggaggttgcgcgtgtggatgcggacagcatgggcgcgacagtgcgcatgtgc
atgtgctgtgtcaaacgtctggtgcgttcagtgtgtccttgcgtgcctcccaccgtacgcagccatcccgcgcgc
ctggaccgtagagaccgcctacgtgtccgctagcggcctcggcctcagcctaagcgccagtagcgccagcgacac
aagcaacactgtcgctaatggcagcagcggcagcagcagcagtcacgagaatgcccgcggccgggagaaagtgct
cctagccgggggccgccgctagctggtttcctcagcgcgtggacggtggtgccttcatcccgaccaccccaggcg
cgtccccagtcccgtcgagctcgcctgccttgtggcccgccttgaccgccctggcgccacccggtggctcgcata
acgactcgctttccgttctccgcctgacgctgtccgcctgacgctctgcgcttgactctttgcgccttcctcccc
tcttcccccag
Mutant sequenced RedAlgaeCHLH DNA (SEQ ID NO: 129):
atgcagacttcctcgcttcttggccggcgcacggcccacccggctgcgggcgcgacgcccaagccggttgcgccc
tcgccccgcgtggctagcacccgccaggtcgcgtgcaatgtggcgactggaccccggccgcccatgaccaccttc
accggtggcaacaagggccctgctaagcagcaggtgtcgctggatctgcgcgacgagggcgctggcatgttcacc
agcaccagcccggagatgcgccgtgtcgtccctgacgatgtgaagggtcgcgttaaggtgaaggttgtgtacgtg
gtgctggaggcccagtaccagtcggccatcagcgctgcggtgaagaacatcaacgccaagaactccaaggtgtgc
ttcgaggtggtgggctacctgctggaggagctgcgtgaccagaagaacctcgatatgctcaaggaggatgtggcc
tctgccaacatcttcatcggctcgctcatcttcattgaggagcttgccgagaagattgtggaggcggtgagcccc
ctgcgcgagaagctggacgcgtgcctgatcttcccgtccatgccggcggtcatgaagctgaacaagctgggcacg
ttttcgatggctcagctgggccagtcgaagtcggtgttctcggagttcatcaagtctgctcgcaagaacaacgac
aacttcgaggagggcttgctgaagctggtgcgcaccctgcctaaggtgctgaagtatctgccctcggacaaggcg
caggacgccaagaacttcgtgaacagcctgcagtactggctgggcggtaactcggacaacctggagaacctgctg
ctgaacaccgtcagcaactacgtgcccgctctgaagggcgtggacttcagcgtggctgagcccaccgcctacccc
gatgtgggtatctggcaccctctggcctcgggcatgtacgaggacctgaaggagtacctgaactggtacgacacc
cgcaaggacatggtcttcgccaaggacgcccccgtcattggcctggtgctgcagcgctcgcacctggtgactggc
gatgagggccactacagcggcgtggtcgctgagctggagagccgcggtgctaaggtcatccccgtctttgccggt
ggcctggacttctccgcccccgtcaagaagttcttctacgaccccctgggctctggccgcacgttcgtggacacc
gttgtgtcgctgaccggcttcgcgctggtgggcggccccgcgcgccaggacgcgccgaaggccattgaggcgctg
aagaacctgaacgtgccctacctggtgtcgctgccgctggtgttccagaccactgaggagtggctggacagcgag
ctgggcgtgcaccccgtccaggtggctctgcaggttgccctgcccgagctggatggtgccatggagcccatcgtg
ttcgctggccgtgactcgaacaccggcaagtcgcactcgctgcccgaccgcatcgcttcgctgtgcgctcgcgcc
gtgaactgggccaacctgcgcaagaagcgcaacgccgagaagaagctggccgtcaccgtgttcagcttcccccct
gacaagggcaacgtcggcactgccgcctacctgaacgtgttcggctccatctaccgcgtgctgaagaacctgcag
cgcgagggctacgacgtgggcgccctgtccgccctcggaggaggatctgatccagtcggtgctgacccagaagga
ggccaagttcaactcgaccgacctgcacatcgcctacaagatgaaggtggacgagtaccagaagctgtgccctta
cgccgaggcgctggaggagaactggggcaagccccccggcaccctgaacaccaacggccaggagctgctggtgta
cggccgccagtacggcaacgtcttcatcggcgtgcagcccaccttcggctacgagggcgacccgatgcgcctgct
gttctcgaagtcggccagcccccaccacggcttcgccgcctactacaccttcctggagaagatcttcaaggccga
cgccgtgctgcacttcggcacccacggctcgctggagttcatgcccggcaagcaggtcggcatgtcgggtgtgtg
ctaccccgactcgctgatcggcaccatccccaacctctactactacgccgccaacaacccgtctgaggccaccat
cgccaagcgccgctcgtacgccaacaccatttcgtacctgacgccgcctgccgagaacgccggcctgtacaaggg
cctgaaggagctgaaggagctgatcagctcgtaccagggcatgcgtgagtctggccgcgccgagcagatctgcgc
caccatcattgagaccgccaagctgtgcaacctggaccgcgacgtgaccctgcccgacgctgacgccaaggacct
gaccatggacatgcgcgacagcgttgtgggccaggtgtaccgcaagctgatggagattgagtcccgcctgctgcc
ctgcggcctgcacgtggtgggctgcccgcccaccgccgaggaggccgtggccaccctggtcaacatcgctgagct
ggaccgcccggacaacaacccccccatcaagggcatgcccggcatcctggcccgcgccattggtcgcgacatcga
gtcgatttacagcggcaacaacaagggcgtcctggctgacgttgaccagctgcagcgcatcaccgaggcctcccg
cacctgcgtgcgcgagttcgtgaaggaccgcaccggcctgaacggccgcatcggcaccaactggatcaccaacct
gctcaagttcaccggcttctacgtggacccctgggtgcgcggcctgcagaacggcgagttcgccagcgccaaccg
cgaggagctgatcaccctgttcaactacctggagttctgcctgacccaggtggtcaaggacaacgagctgggcgc
cctggtagaggcgctgaacggccagtacgtcgagcccggccccggcggtgaccccatccgcaaccccaacgtgct
gcccaccggcaagaacatccacgccctggaccctcagtcgattcccactcaggccgcgctgaagagcgcccgcct
ggtggtggaccgcctgctggaccgcgagcgcgacaacaacggcggcaagtaccccgagaccatcgcgctggtgct
gtggggcactgacaacatcaagacctacggcgagtcgctggcccaggtcatgatgatggtcggtgtcaagcccgt
ggccgacgccctgggccgcgtgaacaagctggaggtgatccctctggaggagctgggccgcccccgcgtggacgt
ggttgtcaactgctcgggtgtgttccgcgacctgttcgtgaaccagatgctgctgctggaccgcgccatcaagct
ggcggccgagcaggacgagcccgatgagatgaacttcgtgcgcaagcacgccaagcagcaggcggcggagctggg
cctgcagagcctgcgcgacgcggccacccgtgtgttctccaacagctcgggctcctactcgtccaacgtcaacct
ggcggtggagaacagcagctggagcgacgagtcgcagctgcaggagatgtacctgaagcgcaagtcgtacgcctt
caactcggaccgccccggcgccggtggcgagatgcagcgcgacgtgttcgagacggccatgaagaccgtggacgt
gaccttccagaacctggactcgtccgagatctcgctgaccgatgtgtcgcactacttcgactccgaccccaccaa
gctggtggcgtcgctgcgcaacgacggccgcacccccaacgcctacatcgccgacaccaccaccgccaacgcgca
ggtccgcactctgggtgagaccgtgcgcctggacgcccgcaccaagctgctcaaccccaagtggtacgagggcat
gcttgcctcgggctacgagggcgtgcgcgagatccagaagcgcatgaccaacaccatgggctggtcggccacctc
gggcatggtggacaactgggtgtacgacgaggccaactcgaccttcatcgaggatgcggccatggccgagcgcct
gatgaacaccaaccccaacagcttccgcaagctggtggccaccttcctggaggccaacggccgcggctactggga
cgccaagcccgagcagctggagcgcctgcgccagctgtacatggacgtggaggacaagattgagggcgtcgaata
a
CHLI1 5′-untranslated region (regulatory region) (SEQ ID NO: 130):
tcctacagagtaaaggtctaggcgatgcgcgactgaaagactgtgaatcccggcgtcgccgtggtgggatgtggg
ccggtgcgctgtcgcagaggataaattacaggtatcaaacaaggttagggcgttggaaggagcggcgctagggaa
ctgaaatcggatctgcatcggaccctcattccgcgacttgtccttcttttgcctcgccccgcagctcttgagttt
tgttcttgaccctttgacacgaaccaaccgatataaaa
CHLI1 3′-untranslated region (regulatory region) (SEQ ID NO: 131):
gcggcaggccttcatggtcgtcgttggagcatttgcggaaaggctgatggcagcagatgcagccatgtcagttgt
ggctgaagttgttggctggggcgggagcgggcagcagctgctgcgagcggccgaagcagcggtgctgctttgcgt
atgagaggaagaccagtgccctcgaggaggcgagtgcctgtgtgagtgtcaggacgtgtgacttcggaaactgag
ggcggtgagtagatgtgactggggcttgcaggaagcctactgaccctatcagaaaaggtgagcaggggtatatgg
tctaggagcgttgccggagcgtggctggccagtgctagccgcgcgggctctgttgctcgctggcgcgccgccgcc
ttcacaacagatgccgtagaaatgcagcgatgtgacgaggcgtggcctattctgcaatgtgtgaggcgccaatgg
cgccactgacaaatggaggagtggtcaaagcttgggtacgttttgagagctgcatcgggcagcgaggatcagtgt
gcggtaagaccgacggcagacggattggcaagggaataggagggacgtgggcgtgggcgcccgcgctttgtcgag
gccgcatgagccggccgcttctagacccgtagcccattttgaacaagcgcccacgcgtgctcccgatgggggaca
tcgatcacgggaattgattaaggggcatgtgtggtgtgcaagtgagtgactggtggttccgtccctgtgaggttg
tttcgttggacgtggctgccgggttgcgcgcgggctaagcgggcctgaggcagagcgctggcgtgtagccgcgag
tatcgatctgtaacgtgc
CHLI1 Exon 1 (SEQ ID NO: 132):
atggccctgaacatgcgtgtttcctcttccaaggtcgctgccaagcagcagggccgcatctccgcggtgccggtt
gtgtcgagcaaggtggcctcctccgcccgcgtggcccccttccag
CHLI1 Exon 2 (SEQ ID NO: 133):
ggcgctcccgtggccgcgcagcgcgctgctctgctgg
CHLI1 Exon 3 (SEQ ID NO: 134):
tgcgcgccgctgccgctactgaggtcaaggctgctgagggccgcactgagaaggagctgg
CHLI1 Exon 4 (SEQ ID NO: 135):
gccaggcccgccccatcttccccttcaccgccatcgtgggccaggatgagatgaagctggcgctgattctgaacg
tgatcgaccccaagatcggtggtgtcatgatcatgggcgaccgtggcactggcaagtccaccaccattcgtgccc
tggcggatctgctgcccgagatgcag
CHLI1 Exon 5 (SEQ ID NO: 136):
gtggttgccaacgacccctttaactcggaccccaccgaccccgagctgatgagcgaggaggtgcgcaaccgcgtc
aaggccggcgagcagctgcccgtgtcttccaagaagattcccatggtggacctgcccctgggcgccactgaggac
cgcgtgtgcggcaccatcgacatcgagaaggcgctgaccgagg
CHLI1 Exon 6 (SEQ ID NO: 137):
gtgtcaaggcgttcgagcccggcctgctggccaaggccaaccgcggcatcctgtacgtggatgaggtcaacctgc
tggacgaccacctg
CHLI1 Exon 7 (SEQ ID NO: 138):
gtcgatgtgctgctggactcggccgcctccggctggaacaccgtggagcgcgagggtatctccatcagccacccc
gcccgcttcatcctggtcggctcgg
CHLI1 Exon 8 (SEQ ID NO: 139):
gcaaccccgaggagggtgagctgcgcccccagctgctggatcgcttcggcatgcacgcccagatcggcaccgtca
aggacccccgcctgcgtgtgcagatcgtgtcgcagcgctcgaccttcgacgagaaccccgccgccttccg
CHLI1 Exon 9 (SEQ ID NO: 140):
caaggactacgaggccggccagatggcgctgacccagcgcatcgtggacgcgcgcaagctgctgaagcagggcga
ggtcaactacgacttccgcgtcaagatcagccagatctgctcggacctgaacgtggacggcatccgcggcgacat
cgtgaccaaccgcgccgccaaggccctggccgccttcgagggccgcaccgag
CHLI1 Exon 10 (SEQ ID NO: 141):
gtgacccccgaggacatctaccgtgtcattcccctgtgcctgcgccaccgcctccggaaagaccccctggctgag
atcgacgacggtgaccgcgtgcgtgagatcttcaagcaggtgttcggcatggagtaa
CHLI1 Intron 1 (SEQ ID NO: 142):
gtgtgcagttgcatctaaagaacgtccaattcatggttactgctcgtggatctaagcggttggctcaccagcgtt
ccatggtccccgattcgtgcacgcag
CHLI1 Intron 2 (SEQ ID NO: 143):
gtgagaagccatgatacaaatataaggatttgaagcggtagatctaggacccatcgaacttgagcaccgacttgc
agtccttgccttgtccggcgactgaacttctgcgcttgctttgcag
CHLI1 Intron 3 (SEQ ID NO: 144):
gtaagtgtcgcgcaaagattttctgccgggacgggtctccctcgcaacatctgaacccatggctcgtttttttgc
cccgcag
CHLI1 Intron 4 (SEQ ID NO: 145):
gtgcgcgcctcccccaaccccagtttggcaaatgtgtggttaagcgtcgaaagcgtgaacagaaacaggtgttgc
gggggccgcggaatggctgcaatgggtgctgggggcttcggagggtctgggggcgagtttgggtatacacgggcg
cgcacacttgaaggaacgctcaaggacgacagcggaggcgtggagacagcgccggcccaagcagcctgtacttgt
agctgctggtcagctgaggcatcacgacttgggaccagcacccggcctcacggttgcacaaggccatcaccgcgc
gccaccacccacgcctcttcaaacccatgccggcacctaccgctacccctgtgacacgctccgcacacgccgccc
cgcacaccccaccatgtgacag
CHLI1 Intron 5 (SEQ ID NO: 146):
gtgagagcgaggcgcggggcgtgctctgcaggctagggtgaagatcaggagagccgaagcgggcccgaacagcgc
agagagaggcaagacgacacccctgccgcgttttgatcacaagattcacacccttgctctccccaacgctcccgc
acatag
CHLI1 Intron 6 (SEQ ID NO: 147):
gtgagcaggggcagataggcggtcgggcggctgggcggcaggggctgtgttggctgtgttgggtgtgggctgagg
ctggtgggtgggctggcgggtggcagggatagcggtgaggggatggtgatggggcagaatgggcgggtgggcgga
cacgtggggtcgttgaagggtgtgtggggacggcaactggtatgcgatatgtcggcttggccctggcggggaaag
cattcgcagaatggcgcacgaacgaggccggggagcgagcggggatgggagacgcaacctgcgctgcgaagtgcg
gcgcgcgctccagttgacacgttgcacgaatgtggccagtgttcgcctgagagttatgggttagaccgccagatg
agccggttaagctggtggtcgcggttgatcggctgcttcccttccggttgcacgcctggcaccctaacattaccc
tgtccgctgctgccctttgcccacag
CHLI1 Intron 7 (SEQ ID NO: 148):
gtgagtgcagctgccgctgcggctgctgatggtgacctgtgcgaccacggggctccgcatttctggacgaagcgt
tgtaccatagccgtcttggtccctgatttgggccggctctggtccgaagccttgacatctacagttcaacatggc
cgtataacgatcctgtgcccacccacacgccaccccgccag
CHLI1 Intron 8 (SEQ ID NO: 149):
gtgagcgcgcgctctacgatacggcagacatgtacacactgcggcgcactgtagagcttgcattgcatttcaagg
cctcgaaagagtagggtggtcgttctctggtggtgtccggccacaattatgcaccccggtgttggtgcagcagct
gtgatgtcacaccttgcatcacccccctactgctgccgcctctcctctcttctcgcccgcag
CHLI1 Intron 9 (SEQ ID NO: 150):
gtgagcagagcaatattgcagagggaagggtggcggaagggtgataacggttggggatctagaggggcgagatgg
atgcacacagcgcggggttggttatgcatgcctgcatggacgcgtgcacgcacccctgatctgccggttttccaa
ctggcgatgccgtattatgacctgcagctcaccatcctcatgcttgatttgcctcgctcag
CHLI1 Protein sequence (SEQ ID NO: 151):
MALNMRVSSSKVAAKQQGRISAVPVVSSKVASSARVAPFQGAPVAAQRAALLVRAAAATEVKAAEGRTEKELGQA
RPIFPFTAIVGQDEMKLALILNVIDPKIGGVMIMGDRGTGKSTTIRALADLLPEMQVVANDPFNSDPTDPELMSE
EVRNRVKAGEQLPVSSKKIPMVDLPLGATEDRVCGTIDIEKALTEGVKAFEPGLLAKANRGILYVDEVNLLDDHL
VDVLLDSAASGWNTVEREGISISHPARFILVGSGNPEEGELRPQLLDRFGMHAQIGTVKDPRLRVQIVSQRSTED
ENPAAFRKDYEAGQMALTQRIVDARKLLKQGEVNYDFRVKISQICSDLNVDGIRGDIVTNRAAKALAAFEGRTEV
TPEDIYRVIPLCLRHRLRKDPLAEIDDGDRVREIFKQVFGME
Mutant protein sequence RedAlgaeCHLH (SEQ ID NO: 152):
MQTSSLLGRRTAHPAAGATPKPVAPSPRVASTRQVACNVATGPRPPMTTFTGGNKGPAKQQVSLDLRDEGAGMFT
STSPEMRRVVPDDVKGRVKVKVVYVVLEAQYQSAISAAVKNINAKNSKVCFEVVGYLLEELRDQKNLDMLKEDVA
SANIFIGSLIFIEELAEKIVEAVSPLREKLDACLIFPSMPAVMKLNKLGTFSMAQLGQSKSVFSEFIKSARKNND
NFEEGLLKLVRTLPKVLKYLPSDKAQDAKNEVNSLQYWLGGNSDNLENLLLNTVSNYVPALKGVDFSVAEPTAYP
DVGIWHPLASGMYEDLKEYLNWYDTRKDMVFAKDAPVIGLVLQRSHLVTGDEGHYSGVVAELESRGAKVIPVFAG
GLDFSAPVKKFFYDPLGSGRTFVDTVVSLTGFALVGGPARQDAPKAIEALKNLNVPYLVSLPLVFQTTEEWLDSE
LGVHPVQVALQVALPELDGAMEPIVFAGRDSNTGKSHSLPDRIASLCARAVNWANLRKKRNAEKKLAVTVFSFPP
DKGNVGTAAYLNVFGSIYRVLKNLQREGYDVGALSALGGGSDPVGADPEGGQVQLDRPAHRLQDEGGRVPEAVPL
RRGAGGELGQAPRHPEHQRPGAAGVRPPVRQRLHRRAAHLRLRGRPDAPAVLEVGQPPPRLRRLLHLPGEDLQGR
RRAALRHPRLAGVHARQAGRHVGCVLPRLADRHHPQPLLLRRQQPV
CHLI1 DNA sequence (SEQ ID NO: 153):
atggccctgaacatgcgtgtttcctcttccaaggtcgctgccaagcagcagggccgcatctccgcggtgccggtt
gtgtcgagcaaggtggcctcctccgcccgcgtggcccccttccagggcgctcccgtggccgcgcagcgcgctgct
ctgctggtgcgcgccgctgccgctactgaggtcaaggctgctgagggccgcactgagaaggagctgggccaggcc
cgccccatcttccccttcaccgccatcgtgggccaggatgagatgaagctggcgctgattctgaacgtgatcgac
cccaagatcggtggtgtcatgatcatgggcgaccgtggcactggcaagtccaccaccattcgtgccctggcggat
ctgctgcccgagatgcaggtggttgccaacgacccctttaactcggaccccaccgaccccgagctgatgagcgag
gaggtgcgcaaccgcgtcaaggccggcgagcagctgcccgtgtcttccaagaagattcccatggtggacctgccc
ctgggcgccactgaggaccgcgtgtgcggcaccatcgacatcgagaaggcgctgaccgagggtgtcaaggcgttc
gagcccggcctgctggccaaggccaaccgcggcatcctgtacgtggatgaggtcaacctgctggacgaccacctg
gtcgatgtgctgctggactcggccgcctccggctggaacaccgtggagcgcgagggtatctccatcagccacccc
gcccgcttcatcctggtcggctcgggcaaccccgaggagggtgagctgcgcccccagctgctggatcgcttcggc
atgcacgcccagatcggcaccgtcaaggacccccgcctgcgtgtgcagatcgtgtcgcagcgctcgaccttcgac
gagaaccccgccgccttccgcaaggactacgaggccggccagatggcgctgacccagcgcatcgtggacgcgcgc
aagctgctgaagcagggcgaggtcaactacgacttccgcgtcaagatcagccagatctgctcggacctgaacgtg
gacggcatccgcggcgacatcgtgaccaaccgcgccgccaaggccctggccgccttcgagggccgcaccgaggtg
acccccgaggacatctaccgtgtcattcccctgtgcctgcgccaccgcctccggaaagaccccctggctgagatc
gacgacggtgaccgcgtgcgtgagatcttcaagcaggtgttcggcatggagtaa

Although the invention has been described with reference to the above examples, it will be understood that modifications and variations are encompassed within the spirit and scope of the invention. Accordingly, the invention is limited only by the following claims.

What is claimed is:

1. A composition comprising a preparation from an algae strain, wherein the algae strain overexpresses or accumulates protoporphyrin IX (PPIX).

2. The composition of claim 1, wherein the preparation is a biomass from the algae strain.

3. The composition of claim 2, wherein the preparation is a fractionated biomass from the algae strain.

4. The composition of claim 3, wherein the fractionated biomass comprises a PPIX-enriched fraction.

5. The composition of claim 4, wherein the PPIX-enriched fraction further comprises a protein-enriched fraction.

6. The composition of claim 1, wherein the preparation is an extracellular fraction of the algae culture.

7. The composition according to any of claims 1-6, wherein the preparation is red or red-like in color.

8. The composition according to any of claims 1-7, wherein the preparation contains a greater amount of PPIX than of heme.

9. The composition according to any of claims 1-8, wherein the preparation contains less than about 1%, 0.5%, 0.1%, 0.05%, 0.01%, 0.005% or 0.001% heme.

10. The composition according to any of claims 1-9, wherein the preparation contains less than about 1%, 0.5%, 0.1%, 0.05%, 0.01%, 0.005% or 0.001% heme-protein.

11. The composition according to any of claims 1-10, wherein the preparation does not contain a detectable amount of heme-protein.

12. The composition according to any of claims 1-11, wherein the preparation does not contain a detectable amount of heme.

13. The composition according to any of claims 1-4, wherein the preparation does not contain a detectable amount of protein.

14. The composition according to any of claims 1-13, wherein the preparation has a protoporphyrin IX content greater than chlorophyll content.

15. The composition according to any of claim 1-4 or 6, wherein the preparation provides at least 5%, 10%, 15%, 20%, 25%, 30%, 35%, 40%, 45%, 50%, 55%, 60%, 65%, 70%, 75%, 80%, 85%, 90%, 95%, 99% or 100% of the total protein content to the edible composition.

16. The composition according to any of claims 1-15, wherein the preparation provides vitamin A, beta carotene or a combination thereof to the composition.

17. The composition of claim 16, wherein the vitamin A, the beta carotene or the combination thereof is at least about 5%, 10%, 15%, 20%, 25%, 30%, 35%, 40%, 45%, 50%, 55%, 60%, 65%, 70%, 75%, 80%, 85%, 90%, 95%, 99% or 100% of the daily recommended requirement.

18. The composition according to any of claims 1-17, wherein the preparation provides less than about 0.01%, 0.05%, 0.1%, 0.2%, 0.3%, 0.4%, 0.5%, 0.6%, 0.7%, 0.8%, 0.9%, 1.0%, 1.2%, 1.5%, 2%, 5% or 10% of total saturated fat present in the composition.

19. The composition according to any of claims 1-18, wherein the preparation provides at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 250 mg, 300 mg, 350 mg, 400 mg, 450 mg or 500 mg of omega-3 fatty acids to the composition.

20. The composition according to any of claims 1-19, wherein the composition has a red or red-like color derived from the preparation.

21. The composition according to any of claims 1-20, wherein the composition has a meat or meat-like flavor derived from the preparation.

22. The composition according to any of claims 1-21, wherein the composition has a meat or meat-like texture derived from the preparation.

23. The composition according to any of claims 1-22, wherein the algae is a Chlamydomonas sp.

24. The composition of claim 23, wherein the algae is Chlamydomonas reinhardtii.

25. The composition of claim 23, wherein the Chlamydomonas sp. is strain CC-125 deposited to the University of Minnesota Chlamydomonas collection center or a derivative thereof.

26. The composition of claim 23, wherein the algae has reduced or an absence of ferrocheletase activity or expression of ferrocheletase.

27. A food product comprising the composition according to any of claims 1-26.

28. The food product of claim 27, wherein the food product comprises a clean meat, a cultured meat, a synthetic meat, a plant-based meat or a non-animal cell-based meat.

29. The food product of any of claims 27-28, wherein the food product is selected from the group consisting of a beef-like food product, a fish-like product, a chicken-like product, a pork-like product and a meat replica.

30. The food product of any of claims 27-29, wherein the food product is vegan, vegetarian or gluten-free.

31. An edible ingredient comprising the composition according to any of claims 1-26.

32. The edible ingredient of claim 31, wherein the edible ingredient is part of a finished product, and wherein the finished product has a red or red-like color derived from the ingredient.

33. The edible ingredient of claim 31, wherein the edible ingredient is part of a finished product, and wherein the finished product has a meat or meat-like flavor derived from the ingredient.

34. The edible ingredient of claim 31, wherein the edible ingredient is part of a finished product, and wherein the finished product has an appearance of blood derived from the ingredient.

35. The edible ingredient according to any of claims 31-34, wherein the finished product is an ingredient for a burger, a sausage, a kebab, a filet, a ground meat-like product or a meatball.

36. The edible ingredient according to any of claims 31-34, wherein the edible composition is part of a finished product and wherein the finished product is an animal feed.

37. The edible ingredient according to any of claims 31-36, wherein the edible ingredient is combined with a protein source, a fat source, a carbohydrate, a starch, a thickener, a vitamin, a mineral, or any combination thereof.

38. The edible ingredient of claim 35, wherein the protein source is textured wheat protein, textured soy protein, fungal protein or algal protein.

39. The edible ingredient according to any of claim 37 or 38, wherein the finished product is free of animal proteins.

40. The edible composition of any of claims 37-39, wherein the fat source comprises at least one of refined coconut oil or sunflower oil.

41. The edible composition of any of claims 37-40, further comprising at least one of potato starch, methylcellulose, water, or a flavor, wherein the flavor is selected at least one of yeast extract, garlic powder, onion powder, and salt.

42. A meat substitute comprising the composition according to any of claims 1-26 or the edible ingredient according to any of claims 31-41.

43. The meat substitute of claim 42, further comprising:
(a) 0.01%-5% (by weight of the meat replica matrix) of a non-animal protoporphyrin IX;
(b) a compound selected from the group consisting of glucose, ribose, fructose, lactose, xylose, arabinose, glucose-6-phosphate, maltose, and galactose, and any combination thereof;
(c) at least 1.5 mM of a compound selected from the group consisting of cysteine, cystine, thiamine, methionine, and any combination thereof; and
(d) one or more proteins selected from the group consisting of plant proteins, fungal proteins and algal proteins,
wherein the meat substitute is a ground beef-like food product that contains no animal product, and wherein cooking the ground beef-like food product results in the production of at least two volatile compounds which have a beef-associated aroma.

44. A method of producing a protoporphyrin IX composition, comprising:
(a) growing an algae population comprising an algae that is a protoporphyrin IX over-producer in a culture; and
(b) isolating a protoporphyrin IX composition from the culture.

45. The method of claim 44, wherein the step of growing comprises culturing the algae in an aerobic fermentation condition.

46. The method of claim 44 or claim 45, wherein the algae contains a chloroplast.

47. The method of claim 46, wherein biosynthesis of the protoporphyrin IX occurs in the chloroplast.

48. The method according to any of claims 44-47, wherein the algae is deficient for its ability to produce chlorophyll.

49. The method according to any of claims 44-48, wherein the algae is deficient for the ability to produce a functional Mg-chelatase enzyme.

50. The method according to any of claims 44-49, wherein the algae is reduced in or lacks one or more of CHLD, CHLI1, CHLI2 or CHLH1.

51. The method according to any of claims 44-50, wherein the algae is reduced in or lacks a functional light dependent protochlorophyllide.

52. The method according to any of claims 44-51, wherein the algae is reduced in or lacks a functional light independent protochlorophyllide.

53. The method according to any of claims 44-52, wherein the algae is reduced in or lacks ChlB, ChlL, or ChlN.

54. The method according to any of claims 44-53, wherein the algae overexpresses one or more of glutamyl-tRNA reductase, glutamate-1-semialdehyde aminotransferase, ALA dehydratase, porphobilinogen deaminase, UPG III synthase, UPG III decarboxylase, CPG oxidase, or PPG oxidase.

55. The method according to any of claims 44-54, wherein the algae is generated by mating to produce a generated strain, and wherein the generated strain is red or red-like in color.

56. The method according to any of claims 44-54, wherein the algae is generated by mutagenesis.

57. The method according to any of claims 44-56, wherein the algae is red or red-like in color.

58. The method according to any of claims 44-57, wherein the isolated protoporphyrin IX composition is an algae biomass.

59. The method of claim 58, wherein the algae biomass is fractionated.

60. The method of claim 59, wherein the algae biomass is fractionated to produce a protein-enriched fraction comprising protoporphyrin IX.

61. The method according to any of claims 44-57, wherein the isolated protoporphyrin IX composition is isolated from extracellular media of the algae culture.

62. The method according to any of claims 44-61, wherein the isolated protopophyrin IX composition is isolated away from algae protein.

63. The method according to any of claims 44-62, wherein the algae is deficient for carotenoids.

64. The method according to any of claims 44-63, wherein the algae is a Chlamydomonas sp.

65. The method according to any of claims 44-64, wherein the algae is a Chlamydomonas reinhardtii.

66. The method of claim 64, wherein the Chlamydomonas sp. is strain CC-125 deposited to the University of Minnesota Chlamydomonas collection center or a derivative thereof.

67. The method according to any of claims 44-66, wherein progeny of a protoporphyrin IX overexpressing algae strain grows faster than its parental strain following a mating with another algae.

68. The method according to any of claims 44-66, wherein the protoporphyrin IX overexpressing algae is a strain produced by mating a strain deficient for carotenoids with a strain exhibiting a red or red-like color.

69. The method according to any of claims 44-66, wherein the protoporphyrin IX overexpressing algae is generated by mutagenesis of a first starting strain and selection of a second strain that grows faster in the dark than the first starting strain.

70. The method according to any of claims 44-66, wherein the protoporphyrin IX overexpressing algae is generated by mutagenesis of a first strain and selection of a second strain that lacks one or more carotenoids from the mutated first strain.

71. The method according to any of claims 44-70, wherein the algae lacks a functional ferrocheletase enzyme.

72. The method according to any of claims 44-70, wherein the algae is reduced in the amount of, or activity of, a ferrocheletase enzyme.

73. The method according to any of claims 44-70, wherein the algae is reduced in the amount of or lacks heme as compared to a wildtype strain.

74. A protoporphyrin IX-containing composition produced by the method according to any of claims 43-70.

75. The composition of claim 74, wherein the composition does not contain a detectable level of heme, heme-binding protein or a combination thereof.

76. The method according to any of claims 44-73, further comprising the steps of: (a) culturing the algae strain under a dark condition, wherein the strain does not produce or is reduced in the production of chlorophyll, and (b) collecting a portion of the algae culture that is red or red-like in color to produce the protoporphyrin IX composition.

77. The method of claim 76, wherein the algae is a Chlamydomonas sp.

78. The method of claim 77, wherein the algae is a Chlamydomonas reinhardtii.

79. The method according to claim 76, wherein the algae exhibits a red or red-like color when grown in the dark condition.

80. The method of claim 76, wherein the collected portion is extracellular media from the algae culture.

81. The method of claim 76, wherein the collected portion is a biomass or fractionated biomass from the algae culture.

82. The method of claim 76, wherein the algae is grown in an aerobic fermentation condition.

83. The method of claim 76, wherein the algae is grown to a density greater than about 10 g/L, 20 g/L, 30 g/L, 40 g/L, 50 g, L, 75 g/L, 100 g/L, 125 g/L, or 150 g/L.

84. The method of claim 76, wherein the algae is grown with acetate as a reduced carbon source.

85. The method of claim 76, wherein the algae is grown with sugar as a reduced carbon source.

86. The method of claim 76, wherein the algae culture is supplemented with iron during the culturing step.

87. The method of claim 76, wherein the algae culture is inoculated at a density greater than about 0.1 g/L, 1.0 g/L, 5.0 g/L, 10 g/L, 20 g/L, 50 g/L, 80 g/L, or 100 g/L.

88. The method of claim 76, further comprising fractionating the collected portion, wherein the fractionating removes from the collected portion substantially all or most of a component selected from the group consisting of carotenoids, starch, and protein.

89. The method of claim 76, further comprising fractionating the collected portion, wherein the fractionating removes from the collected portion substantially all or most of heme, heme-binding protein or a combination thereof.

90. The method of claim 76, further comprising fractionating the collected portion, wherein the fractionating produces a protein-enriched fraction.

91. The method of claim 76, wherein the algae lacks or is reduced in one or more of magnesium chelatase, magnesium protoporphyrinogen IX, protochlorophyllide, chlorophyllide, and chlorophyll.

92. The method of claim 76, wherein the algae lacks or is reduced in ferrocheletase.

93. The method according to any of claim 44-73 or 76-92, wherein the algae is not a transgenic strain.

94. A clean meat product produced by the method according to any of claims 76-93, wherein the method further comprises combining the collected portion with a clean meat manufacturing composition, wherein the collected portion provides a red or red-like color to the clean meat product.

95. The clean meat product of claim 94, wherein the collected portion is a PPIX-enriched fraction or purified PPIX.

96. An edible ingredient produced by the method according to any of claim 44-73 or 76-93, wherein the protoporphyrin IX composition confers a meat or meat-like flavor, texture, odor or any combination thereof to the edible ingredient.

97. The edible ingredient of claim 96, wherein the edible ingredient is incorporated in a finished product selected from the group consisting of a beef-like food product, a fish-like product, a chicken-like product, a pork-like product and a meat replica.

98. The edible ingredient of any of claims 96-97, wherein the edible ingredient is vegan, vegetarian or gluten-free.

99. The edible ingredient of any of claims 96-98, wherein the edible ingredient is free of animal proteins.

100. The edible ingredient of any of claims 96-99, wherein the edible ingredient does not contain any genetically modified components.
